(19) United States
(12) Patent Application Publication
(10) Pub. No.: US 2026/0263363 A1
(43) Pub. Date: Sep. 10, 2026

(54) ADENOVIRAL VECTORS ENCAPSULATED IN CATIONIC LIPOSOMES, AND PREPARATION AND USE THEREOF

(71) Applicants: EpicentRx, Inc., La Jolla, CA (US); The Regents of the University of California, Oakland, CA (US)

(72) Inventors: Christopher LARSON, San Diego, CA (US); Bryan T. ORONSKY, Los Altos Hills, CA (US); Tony R. REID, San Diego, CA (US); Tao DONG, La Jolla, CA (US); Ching-Hsin HUANG, Mountain View, CA (US); Andrew C. KUMMEL, San Diego, CA (US); Abraham T. PHUNG, San Diego, CA (US); Jaimin R. SHAH, San Diego, CA (US); William C. TROGLER, Del Mar, CA (US)

(73) Assignees: EpicentRx, Inc., La Jolla, CA (US); The Regents of the University of California, Oakland, CA (US)

(21) Appl. No.: 18/993,401

(22) PCT Filed: Jul. 12, 2023
(86) PCT No.: PCT/US2023/070079

§ 371 Date: Jan. 10, 2025

Related U.S. Application Data
(60) Provisional application No. 63/368,339, filed on Jul. 13, 2022.

(51) Int. Cl. A61K 9/1271; A61K 9/1277; A61K 35/761; A61P 35/00

(52) U.S. Cl. CPC A61K 9/1271; A61K 9/1277; A61K 35/761; A61P 35/00

(57) ABSTRACT

The present disclosure related to methods for encapsulating a viral particle in a cationic liposome including a cancer cell-targeting moiety. In particular, the present disclosure related to methods for preparing a folate-containing, cationic liposome-encapsulated adenovirus particle, and uses of formulations thereof for cancer therapy.

### CROSS-REFERENCE TO RELATED APPLICATIONS

This application claims priority to and the benefit of U.S. Provisional Application No. 63/368,339, filed Jul. 13, 2022, the disclosure of which is incorporated by reference in its entirety.

### REFERENCE TO ELECTRONIC SEQUENCE LISTING

The contents of the electronic sequence listing (203592002140SEQLIST.xml; Size: 74,813 bytes; and Date of Creation: Jul. 11, 2023) is herein incorporated by reference in its entirety.

### FIELD

The present disclosure related to methods for encapsulating a viral particle in a cationic liposome including a cancer cell-targeting moiety. In particular, the present disclosure related to methods for preparing a folate-containing, cationic liposome-encapsulated adenovirus particle, and uses of formulations thereof for cancer therapy.

### BACKGROUND

Oncolytic viruses are viruses that preferentially replicate in and kill infected cancer cells through lysis. The subsequent release of tumor antigens and their uptake by antigen presenting cells has the potential to activate the immune system and to transform a “cold” tumor microenvironment with few immune effector cells into a “hot” one with increased immune cell infiltration. This potential is theoretically enhanced by the addition of therapeutic transgenes, which are expressed at high levels by the endogenous gene expression machinery of the virus, which depends on viral replication.

The TAV-255 viral vector is a replicating type 5 adenovirus (Ad5) that is selective for tumors. The tumor-selectivity of TAV-255 is neither a product of incorporation of tumor-specific or tissue-specific promoters to control critical viral gene expression nor a product of incorporation of targeting ligands within the viral capsid. Rather tumor-selectivity of TAV-255 is a result of the deletion of a short stretch of DNA, 50 base pairs, in the E1A promoter region that removes key regulatory sequences. The deletion de-targets TAV-255 from normal cells and ablates the native Ad5 tropism for the lungs, eyes, and gastrointestinal tract, but does not interfere with the ability of TAV-255 to replicate within cancer cells, a hallmark of which is regulatory region redundancy. The TAV-255 expression vector armed with a TGF-β trap for binding and neutralizing the ubiquitously overexpressed immunosuppressive cytokine, TGF-β, is called AdAPT-001.

Administration of Ad5, including the TAV-255 viral vector, has been limited to local or regional modes of injection, such as intratumoral injection. Cancer is often a systemic disease and, therefore, a major open question is whether local, intratumoral drug delivery has the potential, outside of control of the injected lesion, to evoke a systemic abscopal effect on distant metastases. To date, the answer to this question with other oncolytic viruses has been “no”, “uncommonly” or “only occasionally” for tumor cells in distant or inaccessible regions. For this reason, combination with other immunotherapies, such as checkpoint inhibitors with their attendant toxicities, is generally required to realize anti-cancer activity.

Intravenous delivery of Ad5 itself is virtually impossible due to the prevalence of preexisting neutralizing antibodies to Ad5, which leads to rapid clearance from the bloodstream and the need for frequent or high-dose administrations. This is costly and may pose potential safety problems, for example, to the liver and spleen where adenoviral sequestration is known to occur.

Another barrier to naked Ad5 infection is that its uptake requires the major receptor for adenoviruses, namely expression of the membrane protein known as Coxsackievirus-Adenovirus Receptor (CAR) (Zhang et al., J. Virol, 79:12125-12131, 2005). This is problematic in utilizing Ad5 for treating cancer because CAR is not uniformly expressed by cancer cells (Wang et al., Chin J Clin Oncol, 34:1150-1153, 2007; and Reeh et al., Br J Cancer, 109:1848-1858, 2013). Cancer cells where CAR expression is absent, weak, or downregulated, which is often the case, demonstrate decreased susceptibility or even refractoriness to adenoviral infection.

One strategy to escape immune recognition and thereby prevent rapid viral clearance is through encapsulation of the negatively charged adenovirus in liposomes or nanoparticles. However, despite promising in vitro data, liposome-encapsulated adenovirus therapies have been limited by the development of in vivo cytotoxicity, low tissue specificity, and poor serum stability.

Critical to the success of oncolytic adenoviral therapy is the development of superior compositions and methods suitable for intravenous delivery to achieve systemic bioavailability, especially in the case of disseminated cancer. As such there is a need in the art for oncolytic adenovirus formulations suitable for targeting the viral load to both primary and metastatic tumor cells to increase adenoviral transduction efficiency.

### BRIEF SUMMARY

The present disclosure related to methods for encapsulating a viral particle in a cationic liposome including a cancer cell-targeting moiety. In particular, the present disclosure related to methods for preparing a folate-containing, cationic liposome-encapsulated adenovirus particle, and uses of formulations thereof for cancer therapy.

An aspect of the disclosure includes methods of preparing a cationic liposome encapsulating an adenovirus particle, the method including: a) preparing a lipid mixture by dissolving 1,2-dioleoyl-3-trimethylammonium-propane (DOTAP), a pegylated carboxylic acid-terminated phospholipid, and a pegylated folate-containing phospholipid in an organic solvent; b) drying the lipid mixture under vacuum to form a dry lipid film; c) hydrating the dried lipid film by contacting the film with a pharmaceutically acceptable buffer to form a liquid suspension including a multilamellar vesicle; d) physically disrupting the multilamellar vesicle to form an empty cationic liposome; and e) adding an adenovirus particle to the empty cationic liposome to prepare a cationic liposome encapsulating the adenovirus particle.

A further aspect of the disclosure includes methods of preparing a cationic liposome encapsulating an adenovirus particle, the method including: a) preparing a lipid mixture by dissolving 1,2-dioleoyl-3-trimethylammonium-propane (DOTAP), a pegylated carboxylic acid-terminated phospholipid, and a pegylated folate-containing phospholipid in an organic solvent; b) drying the lipid mixture under vacuum to form a dry lipid film; c) hydrating the dried lipid film by contacting the film with a pharmaceutically acceptable buffer including an adenovirus particle to form a liquid suspension including a multilamellar vesicle encapsulating the adenovirus particle; and d) physically-disrupting the multilamellar vesicle to prepare a cationic liposome encapsulating the adenovirus particle. In an additional embodiment of this aspect, which may be combined with any of the preceding embodiments or aspects, the lipid mixture further includes cholesterol, and cholesterol is dissolved in the organic solvent along with the DOTAP, the pegylated carboxylic acid-terminated phospholipid, and the pegylated folate-containing phospholipid.

An additional aspect of the disclosure includes methods of preparing a cationic liposome encapsulating an adenovirus particle, the method including: a) preparing a lipid mixture by dissolving 1,2-dioleoyl-3-trimethylammonium-propane (DOTAP), cholesterol, a pegylated carboxylic acid-terminated phospholipid, and a pegylated folate-containing phospholipid in an organic solvent; b) drying the lipid mixture under vacuum to form a dry lipid film; c) hydrating the dried lipid film by contacting the film with a pharmaceutically acceptable buffer to form a liquid suspension including a multilamellar vesicle; d) physically disrupting the multilamellar vesicle to form an empty cationic liposome; and e) adding an adenovirus particle to the empty cationic liposome to prepare a cationic liposome encapsulating the adenovirus particle.

Yet another aspect of the disclosure includes methods of preparing a cationic liposome encapsulating an adenovirus particle, the method including: a) preparing a lipid mixture by dissolving 1,2-dioleoyl-3-trimethylammonium-propane (DOTAP), cholesterol, a pegylated carboxylic acid-terminated phospholipid, and a pegylated folate-containing phospholipid in an organic solvent; b) drying the lipid mixture under vacuum to form a dry lipid film; c) hydrating the dried lipid film by contacting the film with a pharmaceutically acceptable buffer including an adenovirus particle to form a liquid suspension including a multilamellar vesicle encapsulating the adenovirus particle; and d) physically-disrupting the multilamellar vesicle to prepare a cationic liposome encapsulating the adenovirus particle. In a further embodiment of this aspect, which may be combined with any of the preceding embodiments or aspects, in step d) physically-disrupting includes sonicating, extruding, homogenizing, cavitating, or combinations thereof, optionally wherein physically-disrupting includes sonicating the multilamellar vesicle. In still another embodiment of this aspect, which may be combined with any of the preceding embodiments or aspects, in step d) physically-disrupting includes microfluidizing the multilamellar vesicle.

A still further aspect of the disclosure includes methods of preparing a cationic liposome encapsulating an adenovirus particle, the method including: a) preparing a lipid mixture by dissolving 1,2-dioleoyl-3-trimethylammonium-propane (DOTAP), cholesterol, a pegylated carboxylic acid-terminated phospholipid, and a pegylated folate-containing phospholipid in an organic solvent; b) microfluidizing the lipid mixture with a pharmaceutically acceptable buffer in a to form a composition including an empty cationic liposome in an organic solvent; c) evaporating the organic solvent from the composition leaving the empty cationic liposome; and d) adding an adenovirus particle to the empty cationic liposome to prepare a cationic liposome encapsulating the adenovirus particle.

A further aspect of the disclosure includes methods of preparing a cationic liposome encapsulating an adenovirus particle, the method including: a) preparing a lipid mixture by dissolving 1,2-dioleoyl-3-trimethylammonium-propane (DOTAP), cholesterol, a pegylated carboxylic acid-terminated phospholipid, and a pegylated folate-containing phospholipid in an organic solvent; b) microfluidizing the lipid mixture with a pharmaceutically acceptable buffer including an adenovirus particle to form a composition including a cationic liposome encapsulating the adenovirus particle in an organic solvent; c) evaporating the organic solvent from the composition leaving the cationic liposome encapsulating the adenovirus particle.

In an additional embodiment of this aspect, which may be combined with any of the previous embodiments or aspects, the cationic liposome encapsulating the adenovirus particle is a unilamellar vesicle of about 100 to about 900 nm in diameter, optionally about 100 to about 300 nm in diameter. Yet another embodiment of this aspect, which may be combined with any of the previous embodiments or aspects, further includes lyophilizing the cationic liposome encapsulating the adenovirus particle. In still another embodiment of this aspect, which may be combined with any of the previous embodiments or aspects, the pegylated carboxylic acid-terminated phospholipid is a 1,2-diacyl-sn-glycero-3-phosphoethanolamine-N-[carboxy (polyalkylene glycol)-(MW)], or a pharmaceutically acceptable salt thereof, and the pegylated folate-containing phospholipid is a 1,2-diacyl-sn-glycero-3-phosphoethanolamine-N-[folate (polyalkylene glycol)-(MW)], and wherein MW is a molecular weight in the range of about 1000-10000 Daltons, optionally in the range of about 1000-5000 Daltons, optionally about 2000 Daltons. In an additional embodiment of this aspect, the pegylated carboxylic acid-terminated phospholipid is a pharmaceutically acceptable salt of 1,2-distearoyl-sn-glycero-3-phosphoethanolamine-N-[carboxy (polyethylene glycol)-2000] (DSPE-PEG (2000) carboxylic acid), and the pegylated folate-containing phospholipid is a pharmaceutically acceptable salt of 1,2-distearoyl-sn-glycero-3-phosphoethanolamine-N-[folate (polyethylene glycol)-2000] (DSPE-PEG (2000) folate). In yet another embodiment of this aspect, which may be combined with any of the previous embodiments or aspects, the organic solvent includes chloroform. In still another embodiment of this aspect, which may be combined with any of the previous embodiments or aspects, the organic solvent includes methanol, ethanol, isopropanol, or a combination thereof. In a further embodiment of this aspect, which may be combined with any of the previous embodiments or aspects, the pharmaceutically acceptable buffer is a sterile solution with a pH in the range of about 7.0 to 8.0, optionally wherein the pH is from 7.3 to 7.5, optionally wherein the pH is about 7.4. In an additional embodiment of this aspect, the pharmaceutically acceptable buffer is phosphate buffered saline (PBS), tris(hydroxymethyl)aminomethane buffer (Tris), or (4-(2-hydroxyethyl)-1-piperazineethanesulfonic acid buffer (HEPES), optionally wherein the pharmaceutically acceptable buffer is PBS. In still another embodiment of this aspect, which may be combined with any of the preceding embodiments that has cholesterol, the molar ratio of the cholesterol to the DOTAP in step a) is 0.02-0.40 to 1.0 (0.02-0.40:1.0), optionally wherein the molar ratio is 0.26 to 1.0. In a further embodiment of this aspect, which may be combined with any of the previous embodiments or aspects, the molar ratio of the pegylated folate-containing phospholipid to the DOTAP in step a) is 0.01-0.02 to 1.0 (001-0.02:1.0), optionally wherein the molar ratio is 0.01 to 1.0. In yet another embodiment of this aspect, which may be combined with any of the previous embodiments or aspects, the molar ratio of the pegylated carboxylic acid-terminated phospholipid to the DOTAP in step a) is 1.0 to 0.02-0.04 (1.0:02-0.04), optionally wherein the molar ratio is 1.0 to 0.02. In still another embodiment of this aspect, which may be combined with any of the previous embodiments or aspects, the molar ratio of the DOTAP, the cholesterol, the pegylated carboxylic acid-terminated phospholipid, and the pegylated folate-containing phospholipid in step a) is about 1.0:0.26:0.02:0.01. In an additional embodiment of this aspect, which may be combined with any of the previous embodiments or aspects, the adenovirus particle to DOTAP lipid ratio is about 5×10⁶ to about 5×10⁸ viral particles: nmol (vp/nM). In another embodiment of this aspect, the adenovirus particle to DOTAP lipid ratio is about 1×10⁷ to about 1×10⁸ vp/nM, optionally wherein the ratio is about 5×10⁷ vp/nM, or wherein the ratio is 5.17×10⁷ vp/nM. In a still further embodiment of this aspect, which may be combined with any of the previous embodiments or aspects, the adenovirus is a recombinant human adenovirus of serotype 5 (Ad5).

In yet another embodiment of this aspect, which may be combined with any of the previous embodiments or aspects, the adenovirus is an oncolytic adenovirus. In a further embodiment of this aspect, the oncolytic adenovirus includes a modified Ela regulatory sequence in which at least a portion of a at least one Pea3 and/or E2F transcription factor binding site is deleted. In an additional embodiment of this aspect, the at least one transcription factor binding site is selected from Pea3 I, E2F I, Pea3 II, E2F II, Pea3 III, Pea3 IV, Pea3 V, and combinations thereof. In yet another embodiment of this aspect, the oncolytic adenovirus includes a deletion of nucleotides located upstream of the Ela transcription initiation site, wherein the deletion optionally includes a deletion of: nucleotides −393 to −304, nucleotides −305 to −255, nucleotides −270 to −240, nucleotides −299 to −293, nucleotides −270 to −265, nucleotides −299 to −293, or nucleotides −270 to −265. In still another embodiment of this aspect, the deletion includes a deletion of nucleotides corresponding to 195-244 of the Ad5 genome (SEQ ID NO: 1). In a further embodiment of this aspect, the oncolytic adenovirus includes a modified TATA box-based promoter operably linked to a gene, wherein the modified TATA box-based promoter lacks a functional TATA box and permits selective expression of the gene in a hyperproliferative cell and/or a modified CAAT box-based promoter operably linked to a gene, wherein the modified CAAT box-based promoter lacks a functional CAAT box and permits selective expression of the gene in a hyperproliferative cell. In an additional embodiment of this aspect, the modified TATA box-based promoter is an E1a promoter, E1b promoter, or E4 promoter. In still another embodiment of this aspect, the modified TATA box-based promoter is an E1a promoter. In a further embodiment of this aspect, which may be combined with any of the preceding embodiments that has a modified TATA box-based promoter, the modification included in the modified TATA box-based promoter includes a deletion of the entire TATA box. In yet another embodiment of this aspect, which may be combined with any of the preceding embodiments that has a modified TATA box-based promoter, the oncolytic adenovirus includes a deletion of nucleotides corresponding to −27 to −24 of the E1a promoter, to −31 to −24 of the E1a promoter, to −44 to +54 of the E1a promoter, to 146 to +54 of the E1a promoter, to 472 to 475 of the Ad5 genome (SEQ ID NO: 1), to 468 to 475 of the Ad5 genome (SEQ ID NO: 1), to 455 to 552 of the Ad5 genome (SEQ ID NO: 1), to 353 to 552 of the Ad5 genome (SEQ ID NO: 1), or to 477 to 484 of the Ad35 genome (SEQ ID NO: 3). In still another embodiment of this aspect, the modified CAAT box-based promoter is an E1a promoter, E1b promoter, or E4 promoter. In yet another embodiment of this aspect, the modified CAAT box-based promoter is an E1a promoter. In a further embodiment of this aspect, which may be combined with any of the preceding embodiments that has a modified CAAT box-based promoter, the modification included in the modified CAAT box-based promoter includes a deletion of the entire CAAT box. In yet another embodiment of this aspect, which may be combined with any of the preceding embodiments that has a modified CAAT box-based promoter, the oncolytic adenovirus includes a deletion of nucleotides corresponding to −76 to −68 of the E1a promoter. In a further embodiment of this aspect, which may be combined with any of the preceding embodiments that has an oncolytic adenovirus, the oncolytic adenovirus includes at least one transgene, wherein insertion sites of the at least one transgene are selected from an E1b-19K insertion site, an E3 insertion site, an E4 insertion site, an IX-E2 insertion site, an L5-E4 insertion site, and combinations thereof.

An additional aspect of the disclosure includes a pharmaceutical formulation including an excipient, and a plurality of the cationic liposome-encapsulated adenovirus particles produced by the method of any one of the preceding embodiments or aspects. Another embodiment of this aspect further includes a serum albumin. In a further embodiment of this aspect, the serum albumin is a human serum albumin.

Yet another aspect of the disclosure includes methods of treating a cancer in a patient, the method including administering an effective mount of the pharmaceutical formulation of any one of the preceding embodiments to the patient to treat the cancer. In an additional embodiment of this aspect, the cancer is a carcinoma. In a further embodiment of this aspect, the carcinoma is an adenocarcinoma or a squamous cell carcinoma. In still another embodiment of this aspect, which may be combined with any of the preceding embodiments, cells of the cancer are coxsackievirus-adenovirus receptor (CAR) negative. In yet another embodiment of this aspect, which may be combined with any of the preceding embodiments, cells of the cancer are folate receptor positive. In a further embodiment of this aspect, which may be combined with any of the preceding embodiments, cells of the cancer are folate receptor negative. In a further embodiment of this aspect, which may be combined with any of the preceding embodiments, the pharmaceutical formulation is administered by parenteral delivery. In an additional embodiment of this aspect, the pharmaceutical formulation is administered by intravenous, intraarterial or subcutaneous injection. In yet another embodiment of this aspect, the pharmaceutical formulation is administered by intratumoral or intranodal delivery.

Still another aspect of the disclosure includes methods of determining margins of a tumor in a surgical patient, the method including: a) administering an effective mount of the pharmaceutical formulation of any one of claims 30-32 to the surgical patient; b) allowing time for the adenovirus particles to enter into and replicate within cells of the tumor thereby producing a plurality of adenovirus progeny; and c) detecting the adenovirus progeny thereby determining margins of the tumor, wherein the adenovirus particles express a detectable protein when replicating within cells of the tumor. In an additional embodiment of this aspect, the detectable protein is a fluorescent protein that is detected in the adenovirus progeny when exposed to light in the appropriate wavelength range. In a further embodiment of this aspect, the fluorescent protein is a green fluorescence protein and the appropriate wavelength range and the fluorescent protein is in the blue to ultraviolet range. In still another embodiment of this aspect, which may be combined with any of the preceding embodiments, step c) takes place from 1 to 7 days after step a), optionally wherein step c) takes place from about 24 to 72 hours after step a).

A further aspect of the disclosure includes a pharmaceutical formulation including an excipient, and a plurality of cationic liposome-encapsulated adenovirus particles, wherein the cationic liposome is a large unilamellar vesicle including 1,2-dioleoyl-3-trimethylammonium-propane (DOTAP), a pegylated carboxylic acid-terminated phospholipid, and a pegylated folate-containing phospholipid, and wherein each of the cationic liposome-encapsulated adenovirus particles is about 100 to about 900 nm in diameter, optionally about 100 to about 300 nm in diameter. In an additional embodiment of this aspect, the large unilamellar vesicle further includes cholesterol. In yet another embodiment of this aspect, which may be combined with any of the preceding embodiments, the pegylated carboxylic acid-terminated phospholipid is a 1,2-diacyl-sn-glycero-3-phosphoethanolamine-N-[carboxy (polyalkylene glycol)-(MWa)], or a pharmaceutically acceptable salt thereof, and the pegylated folate-containing phospholipid is a 1,2-diacyl-sn-glycero-3-phosphoethanolamine-N-[folate (polyalkylene glycol)-(MWb)], and wherein MWa is a molecular weight in the range of about 1000-10000 Daltons, and optionally MWb is a molecular weight in the range of about 1000-5000 Daltons, optionally about 2000 Daltons. Still another embodiment of this aspect, which may be combined with any of the preceding embodiments, further includes a serum albumin, optionally wherein the serum albumin is human serum albumin. In a further embodiment of this aspect, which may be combined with any of the preceding embodiments, the adenovirus is an oncolytic adenovirus, optionally wherein the oncolytic adenovirus is a recombinant human adenovirus of serotype 5 (Ad5). In yet another embodiment of this aspect, the oncolytic adenovirus includes a modified E1a regulatory sequence in which at least a portion of a at least one Pea3 and/or E2F transcription factor binding site is deleted. In still another embodiment of this aspect, the at least one transcription factor binding site is selected from Pea3 I, E2F I, Pea3 II, E2F II, Pea3 III, Pea3 IV, Pea3 V, and combinations thereof. In a further embodiment of this aspect, the oncolytic adenovirus includes a deletion of nucleotides located upstream of the E1a transcription initiation site, wherein the deletion optionally includes a deletion of: nucleotides −393 to −304, nucleotides −305 to −255, nucleotides −270 to −240, nucleotides −299 to −293, nucleotides −270 to −265, nucleotides −299 to −293, or nucleotides −270 to −265. In an additional embodiment of this aspect, the deletion includes a deletion of nucleotides corresponding to 195-244 of the Ad5 genome (SEQ ID NO: 1). In yet another embodiment of this aspect, the oncolytic adenovirus includes a modified TATA box-based promoter operably linked to a gene, wherein the modified TATA box-based promoter lacks a functional TATA box and permits selective expression of the gene in a hyperproliferative cell and/or a modified CAAT box-based promoter operably linked to a gene, wherein the modified CAAT box-based promoter lacks a functional CAAT box and permits selective expression of the gene in a hyperproliferative cell. In still another embodiment of this aspect, the modified TATA box-based promoter is an E1a promoter, E1b promoter, or E4 promoter. In a further embodiment of this aspect, the modified TATA box-based promoter is an E1a promoter. In an additional embodiment of this aspect, which may be combined with any of the preceding embodiments that has a TATA box-based promoter, the modification included in the modified TATA box-based promoter includes a deletion of the entire TATA box. In yet another embodiment of this aspect, which may be combined with any of the preceding embodiments that has a TATA box-based promoter, the oncolytic adenovirus includes a deletion of nucleotides corresponding to −27 to −24 of the E1a promoter, to −31 to −24 of the E1a promoter, to −44 to +54 of the E1a promoter, to 146 to +54 of the E1a promoter, to 472 to 475 of the Ad5 genome (SEQ ID NO: 1), to 468 to 475 of the Ad5 genome (SEQ ID NO: 1), to 455 to 552 of the Ad5 genome (SEQ ID NO: 1), to 353 to 552 of the Ad5 genome (SEQ ID NO: 1), or to 477 to 484 of the Ad35 genome (SEQ ID NO: 3). In still a further embodiment of this aspect, the modified CAAT box-based promoter is an E1a promoter, E1b promoter, or E4 promoter. In an additional embodiment of this aspect, the modified CAAT box-based promoter is an E1a promoter. In yet another embodiment of this aspect, which may be combined with any of the preceding embodiments that has a CAAT box-based promoter, the modification included in the modified CAAT box-based promoter includes a deletion of the entire CAAT box. In still another embodiment of this aspect, which may be combined with any of the preceding embodiments that has a CAAT box-based promoter, the oncolytic adenovirus includes a deletion of nucleotides corresponding to −76 to −68 of the E1a promoter. In a further embodiment of this aspect, which may be combined with any of the preceding embodiments that has an oncolytic adenovirus, the oncolytic adenovirus includes at least one transgene, wherein insertion sites of the at least one transgene are selected from an E1b-19K insertion site, an E3 insertion site, an E4 insertions site, an IX-E2 insertion site, an L5-E4 insertion site, and combinations thereof.

Yet another aspect of the disclosure includes methods of transfecting mammalian cells, the method including: a) contacting a mammalian cell with an effective amount of the pharmaceutical formulation of any one of claims 40-42 or any one of claims 56-74; and b) culturing the mammalian cell in culture media under conditions suitable for the adenovirus particle to enter into and replicate within cells of the tumor thereby producing a plurality of adenovirus progeny. In an additional embodiment of this aspect, multiplicity of infection of step a) is about 12.5 to 50 adenovirus particles per mammalian cell. In a further embodiment of this aspect, which may be combined with any of the preceding embodiments, the mammalian cells are coxsackievirus-adenovirus receptor (CAR) negative. In still another embodiment of this aspect, which may be combined with any of the preceding embodiments, the mammalian cells are folate receptor positive. An additional embodiment of this aspect, which may be combined with any of the preceding embodiments, further includes isolating the adenovirus progeny.

### BRIEF DESCRIPTION OF THE DRAWINGS

FIG. 1 illustrates that liposomal encapsulation enhances the transfection efficiency in CAR-negative (MCF7, CT26, and THP-1; bottom row, indicated by (c), (d), and (e) respectively) and CAR-positive (HEK293 and A549; top row, indicated by (a) and (b)) cells. GFP⁺ cells were counted under a Keyence BZ-X710 fluorescence microscope on day 2, 3, 4, 5, 6 for HEK293, A549, MCF7, THP-1, and CT26, respectively.

FIGS. 2A-2B illustrate that encapsulation enhances the transfection of CAR-negative cells. FIG. 2A shows a comparison of anti-GFP staining of GFP-AD transduced CT26 cells between the control PBS (a), unencapsulated GFP-AD at moi 100 (b), liposome-encapsulated GFPAD-DF at moi 6.5 (c), and GFPAD-DF at moi 100 (d). FIG. 2B shows quantification of stained GFP-positive cells based on the IHC slides (e). Mean GFP positive cells based on IHC images of n=12 and standard deviations are shown for moi 100; scale bar=50 μm, ****p<0.0001.

FIGS. 3A-3B illustrate that encapsulated virus efficiently transfects primary breast tumor cells. FIG. 3A shows transfection percentage as GFP-positive cell percentage. GFP-positive cells were counted under a Keyence BZ-X710 fluorescence microscope on day 4 after infection, and ** means p<0.01 and *** means p<0.001. FIG. 3B illustrates that primary breast tumor cells are predominantly CAR negative cells.

FIG. 4 illustrates that liposome-encapsulated virus replicates in CAR negative CT26 cells. Hexon expression of non-encapsulated and encapsulated TAV255 in transfected CT26 cells is shown as the mean with standard errors of the mean.

FIG. 5 illustrates the viability of replicating virus encapsulated in liposomes in CAR-positive (A549; top row) and CAR-negative cells (CT26; bottom row). Cell viability was measured on day 2, 4, 6 post-infection using an Alamar Blue assay. Data are shown as the mean with standard errors of the mean.

FIG. 6 illustrates that encapsulated adenovirus suppresses tumor growth. In the top row ((a)-(d)), vehicle (PBS) or adenovirus (n=5-10) were intratumorally-injected every other day, and infiltrating lymphocytes in the tumor microenvironment were analyzed by flow cytometry. In the bottom row ((e)-(g)), tumor progression curves are shown for tumors injected with: PBS (vehicle, n=3); naked adenovirus (TAV-255, n=6), or encapsulated adenovirus ( 1/10×TAV255-Df, n=6). In the rightmost bottom row (h), a survival curve is shown comparing treatment with PBS (vehicle, n=8), naked adenovirus (TAV255, n=6), and encapsulated adenovirus ( 1/10×TAV255-Df, n=6). Data are shown as the mean with standard errors of the mean; * means p<0.05 and ** means p<0.01.

FIG. 7 is a cartoon illustrating transfection of both coxsackievirus-adenovirus receptor (CAR)-positive cells and CAR-negative tumor cells by encapsulated adenovirus. In contrast, naked adenovirus readily transfects CAR-positive cells, but not CAR-negative tumor cells.

FIGS. 8A-8B are cartoons illustrating two processes for synthesis of adenovirus encapsulated DOTAP-folate liposome (Ad-Df). FIG. 8A shows Process 1, which involves production of liposomes in the presence of Ad. FIG. 8B shows Process 2, which involves mixing Ad with pre-formed liposomes.

FIG. 9 is a cartoon illustrating cell entry by naked adenovirus (Naked Ad) and adenovirus encapsulated in a DOTAP-folate liposome (Ad-Df). The left panel shows that adenovirus (Ad) uses the coxsackie and adenovirus receptor (CAR) to enter into host cells. Ad does not efficiently enter and transfect cells that express low to no CAR. Tumors have various levels of CAR expression and many tumor cells are resistant to Ad-based gene transfer because of low CAR expression. The central panel shows that Ad-Df can enter cells through endocytosis or membrane fusion when the target cells do not express CAR. Therefore, Ad-Df is capable of transfecting both CAR-positive and CAR-negative cells. The right panel shows that Ad-Df entry into folate receptor (FR)-positive cells is enhanced by FR-mediated endocytosis. The FR is commonly expressed or even overexpressed on tumor cells, including breast, lung, colorectal, and ovarian cancers.

FIGS. 10A-10B illustrate two microfluidization processes for production of liposome-encapsulated adenoviruses from rehydrated lipid films. In the process shown in FIG. 10A, adenovirus encapsulation is accomplished by contacting empty liposomes with adenovirus. In the process shown in FIG. 10B, adenovirus encapsulation is accomplished by formation of liposomes in the presence of adenovirus. For long term stability, storage of empty liposomes is preferable to storage of encapsulated adenovirus.

FIGS. 11A-11D illustrate the effects of different treatments on mice over time. FIG. 11A is a Kaplan-Meier plot showing survival of mice in three treatment groups over time. There were 6-8 mice in each group: 1) mice in the control group were treated with PBS; 2) mice in the TAV group were treated with naked adenovirus; and 3) mice in the DfTAV group were treated with adenovirus encapsulated in folate containing liposomes. FIGS. 11B-11D show a comparison of unencapsulated vs. encapsulated TAV255 on CAR-deficient tumor growth and remission. The treatment schedule: PBS, empty liposome (Df), TAV255, or TAV255-Df (n=10-24) were intratumorally injected every other day. Survivors with full remission were rechallenged by CT-26 tumors on day 64. FIG. 11B is a Kaplan-Meier plot showing survival of mice in four treatment groups over time (Vehicle n=10; empty liposomes (Df) n=10; unencapsulated adenovirus TAV255 n=24; and liposomes encapsulated TAV255-Df n=24) with initial challenge of CT-26 tumor on the right flank of the mice and rechallenge of the tumors on the left flank. FIG. 11C shows the percent of the mice from the treatment groups that were tumor free at day 60. 33% of the mice had complete remission from the treatment with TAV255, and 58% of the mice had complete remission from treatment with TAV255-Df. FIG. 11D shows the percent of the mice from the treatment groups that were tumor free from rechallenge. 8/8 and 13/13 mice from tav255 and tav255-df treatments survived from the rechallenge. No mice from the control group survived rechallenge.

FIGS. 12A-12B show the effect of liposome encapsulation of protection and transfection efficiency of adenovirus. FIG. 12A shows the effect of liposome encapsulation on protection of adenovirus from neutralizing antibodies. FIG. 12B shows the effect of liposome encapsulation on transfection efficiency of adenovirus into CAR-positive cells (HEK293) in the presence neutralizing antibodies.

FIG. 13 shows that encapsulation of adenovirus protects from neutralizing antibody present in the circulation. Mice were immunized with 5e8 adenoviral particles in PBS given by subcutaneous injection. Control, unimmunized mice, were treated only with subcutaneous injection of PBS. Three weeks later, mice were given an IV injection of unencapsulated or encapsulated adenovirus expressing GFP. Blood from the mice was collected 5 minutes after injection of the unencapsulated or encapsulated virus, the serum was isolated and used to transfect HEK293 cells and expression of GFP was determined for each group. The results demonstrate that in unimmunized mice, both unencapsulated and encapsulated virus demonstrate statistically similar expression of GFP. In contrast, immunization significantly reduces GFP expression from virus isolated from the blood of mice treated with unencapsulated adenovirus but not from mice treated with encapsulated adenovirus. These results demonstrate that encapsulation of adenovirus protects from antibody mediated neutralization permitting sustained levels of biologically active adenovirus in the systemic circulation.

FIG. 14 shows that encapsulation of adenovirus protects from neutralizing antibody present in the circulation and results in tumor cell transduction following IV administration. Car negative (ct-26) cells were implanted in mice and tumors were allowed to grow to approximately 500 mm3. Mice (8 per group) were then treated with unencapsulated or encapsulated virus with 1.5e10 viral particles for 6 consecutive days. 24 hours after the final IV treatment, tumors were harvested and RT-qPCR was used to determine the expression of the E1a viral mRNA in the tumors. Significantly higher levels of E1a mRNA expression were observed with encapsulation of adenovirus compared to unencapsulated adenovirus.

FIGS. 15A-15B show immunofluorescence staining of CD8+ T cells. FIG. 15A shows DAPI-stained cells in dark grey and CD8+ cells in white. FIG. 15B shows quantification of the CD8+ T cells in both primary ((a), on left) and secondary ((b), on right) tumors based on immunofluorescent staining (n=3-6). Data are shown as mean with standard errors of the mean (SEM); scale bar=10 μm; ns=no significant difference; *p value<0.05; **p value<0.01; ***p value<0.001, ****p value<0.0001.

### DETAILED DESCRIPTION

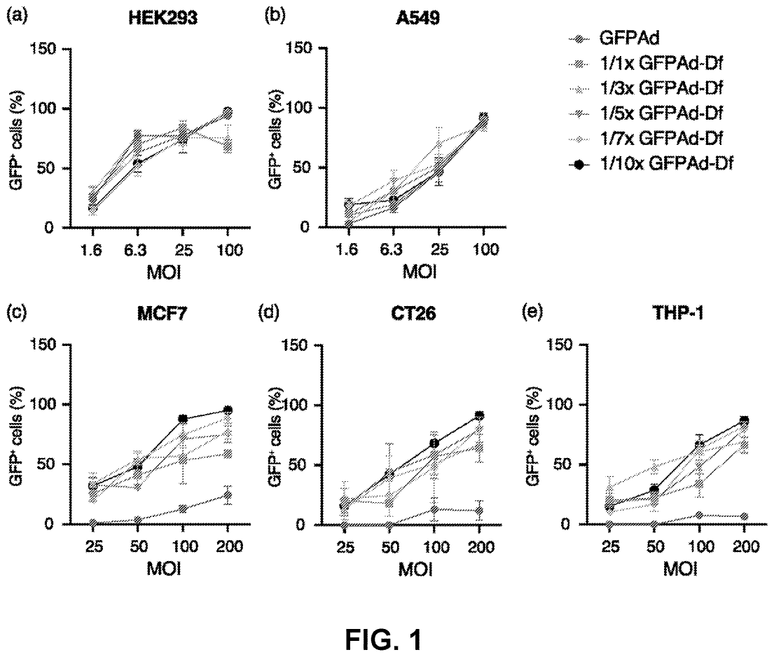

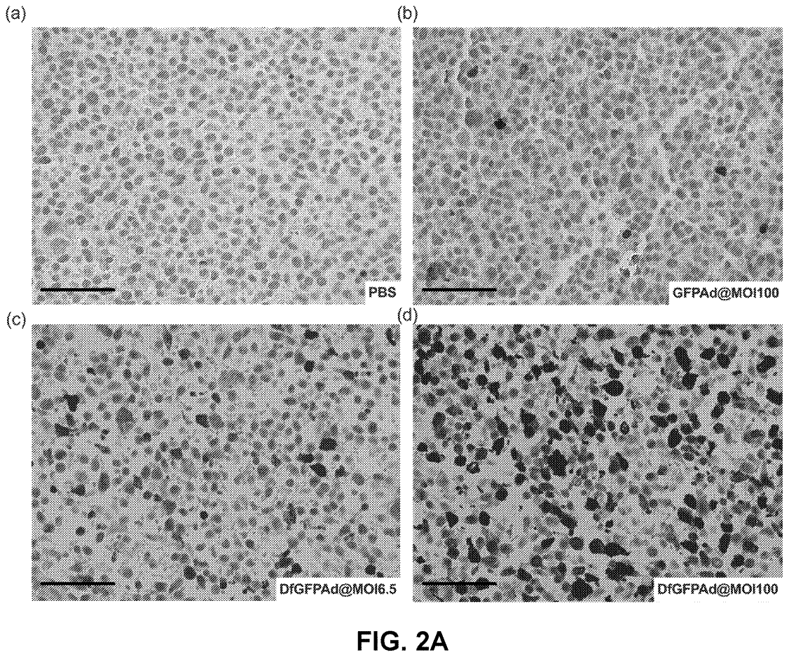

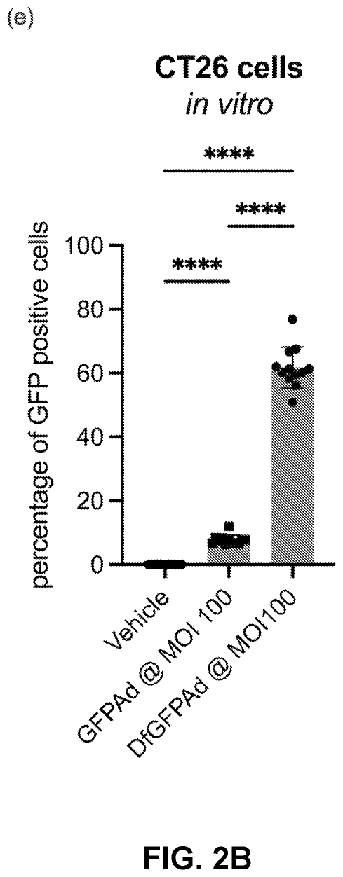

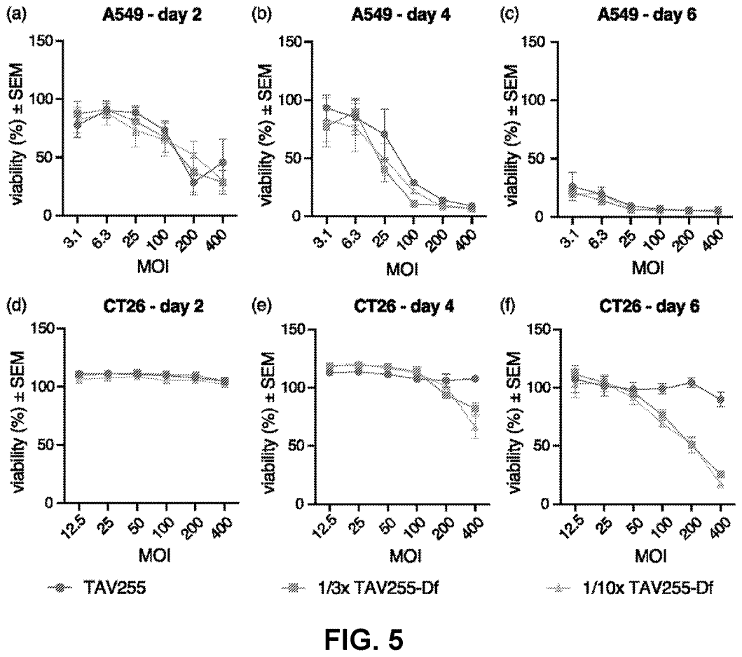

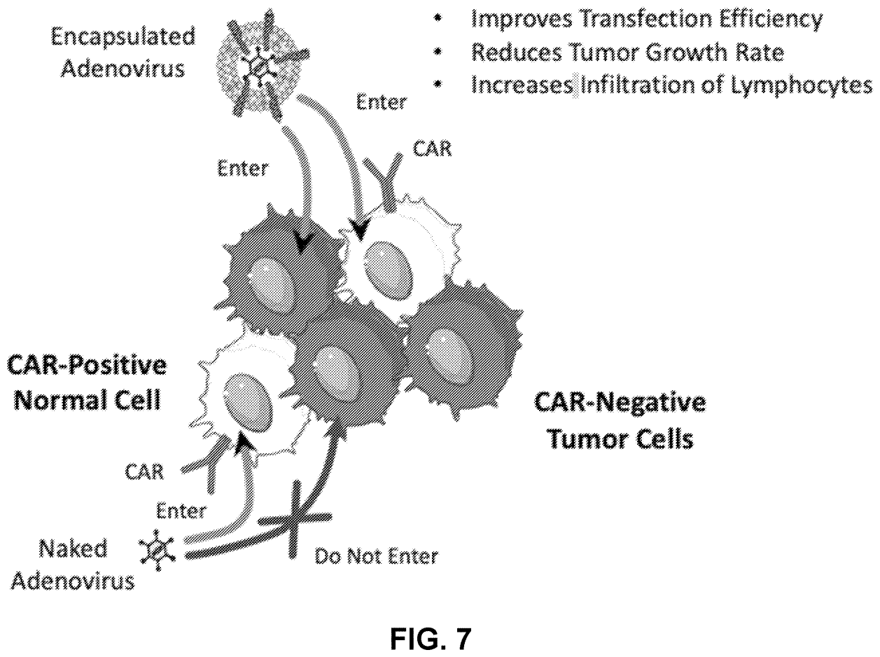

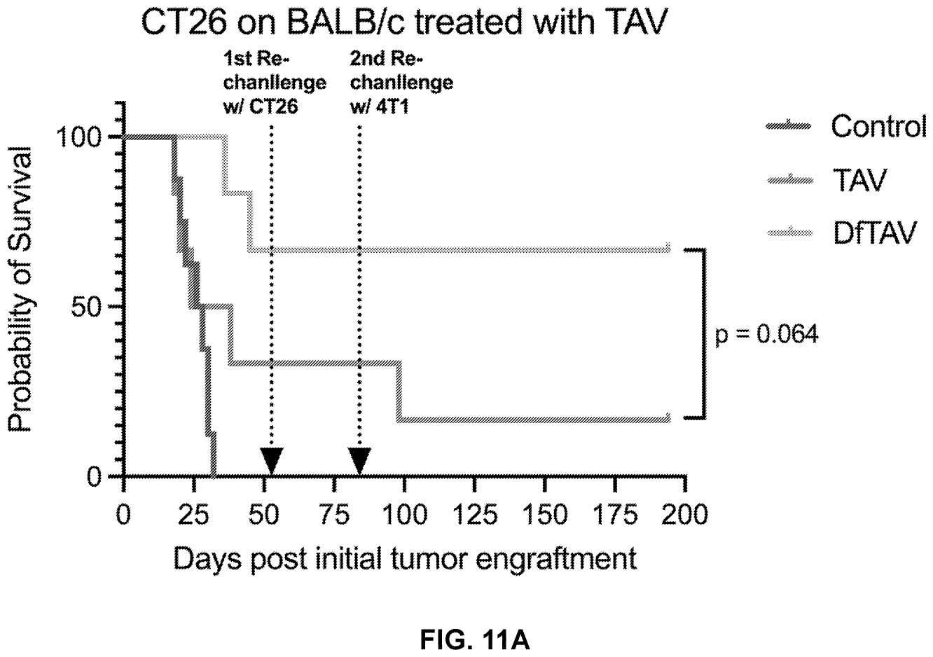

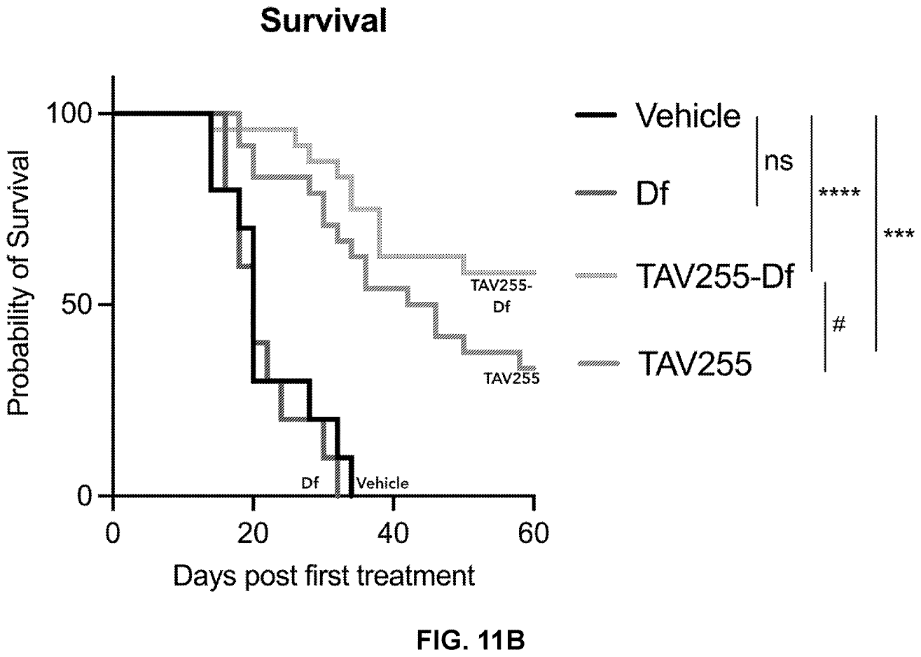

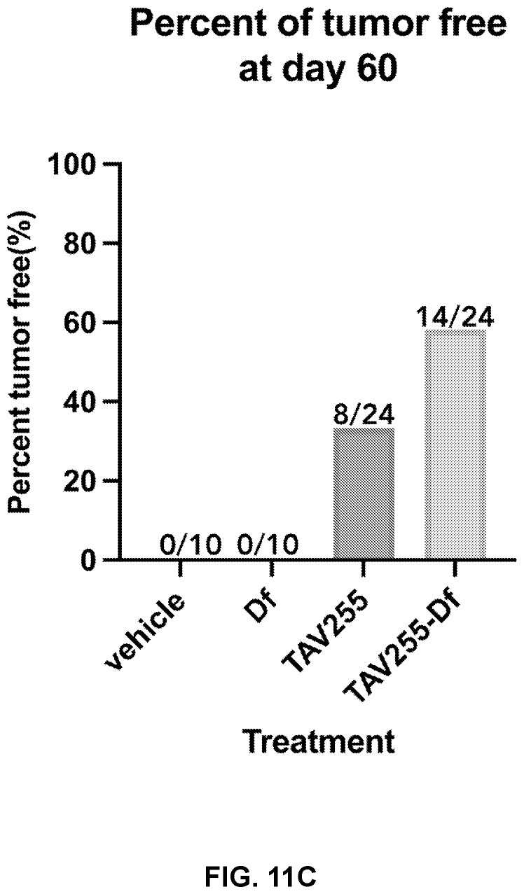

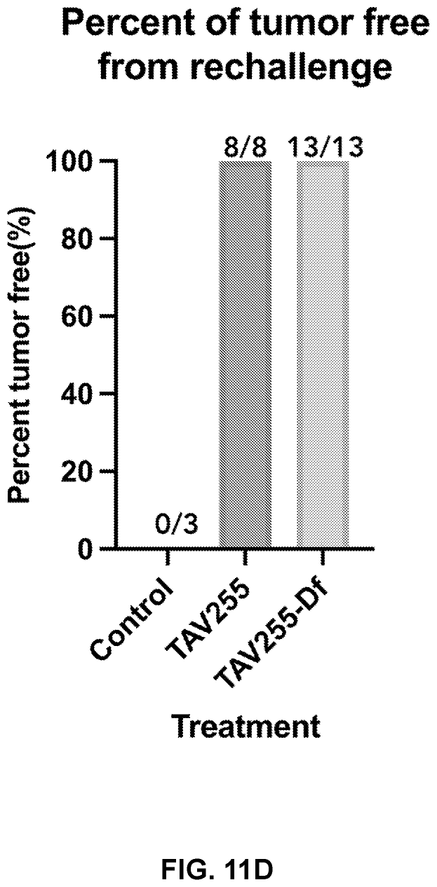

Adenovirus (Ad)-based vectors have shown considerable promise for gene therapy. However, Ad requires the coxsackievirus and adenovirus receptor (CAR) to enter cells efficiently and low CAR expression is found in many human cancers, which hinder adenoviral gene therapies. Here, cationic 1,2-dioleoyl-3-trimethylammonium-propane (DOTAP)-folate liposomes (Df) encapsulated replication-deficient Ad were synthesized and showed improved transfection efficiency in various CAR-deficient cell lines, including epithelial and hematopoietic cell types. A cartoon shown adenoviral uptake into CAR-negative tumor cells and CAR-positive normal cells is shown in FIG. 7. When encapsulating replication-competent oncolytic Ad (TAV255) in DOTAP-folate liposome (TAV255-Df), the adenoviral structural protein, hexon, was readily produced in CAR-deficient cells and the tumor cell killing ability was 5× higher than that of the non-encapsulated adenovirus in CAR-deficient CT-26 tumor cells (FIG. 5). In CAR-deficient CT26 colon carcinoma murine models, replication-competent TAV255-Df treatment of subcutaneous tumors by intratumoral injection resulted in 67% full tumor remission, prolonged survival, and anti-cancer immunity when mice were re-challenged with cancer cells in the absence of further treatment (FIGS. 11A-11D). The preclinical data of Example 1 shows that DOTAP-folate liposomes significantly enhance the transfection efficiency of Ad in CAR-deficient cells (FIG. 1).

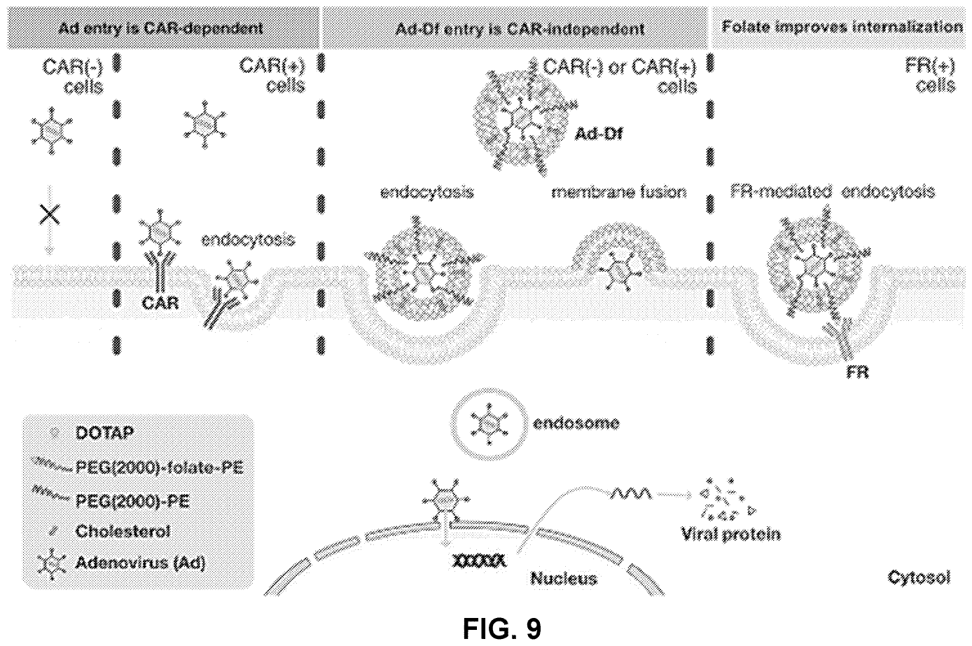

Ad was encapsulated in positively charged DOTAP-folate liposomes (Ad-Df in FIG. 9, left panel) to facilitate viral attachment to negatively charged cell membranes and, thus, to eliminate the need of CAR for Ad cell entry (FIG. 9, middle panel). The cationic liposome and Ad assembly can enter cells through endocytosis and membrane fusion that facilitate the Ad internalization without a need for CAR, thereby enhancing transfection (FIG. 9, right panel). Folate-modified lipids in the liposome formula can further improve cellular uptake of Ad-Df by attaching to the folate receptor (FR) on the targeted cells, leading to increased adenoviral transfection efficiency (FIG. 9). Ad-Df particles were shown to substantially improve the adenoviral transgene expression in CAR-negative cells. Improvement was observed in transfecting both epithelial cell lines and hematopoietic cells at a high and a low multiplicity of infection (MOI). Compared to a commercial transfection reagent (Lipofectin® marketed by ThermoFisher) that requires a MOI=500 to transfect 40% CAR-negative CT26 cells, Ad-Df needs 10× lower MOI to reach the same transfection efficiency. When treating hematopoietic cells, the Ad-Df formulation can transfect a higher fraction of hematopoietic cells than other encapsulation techniques at a MOI=200, resulting in a comparative decrease of the transfection failure fraction from 33% to 13% (Buttgereit et al., Caner Gene Ther, 7 (8): 1145-1155, 2000).

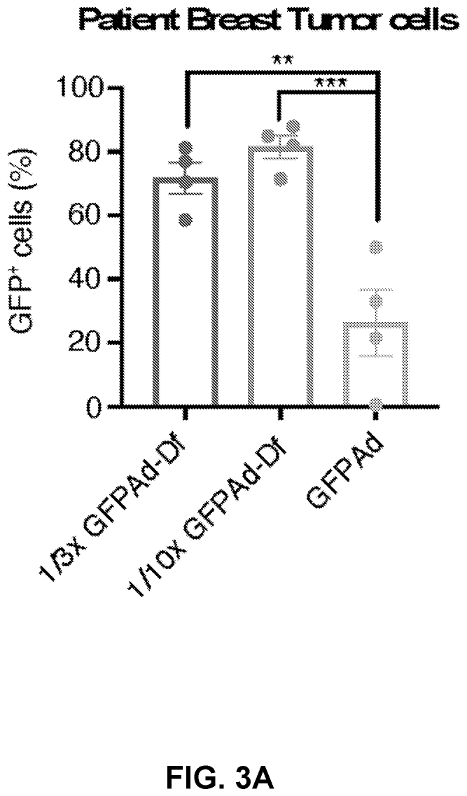

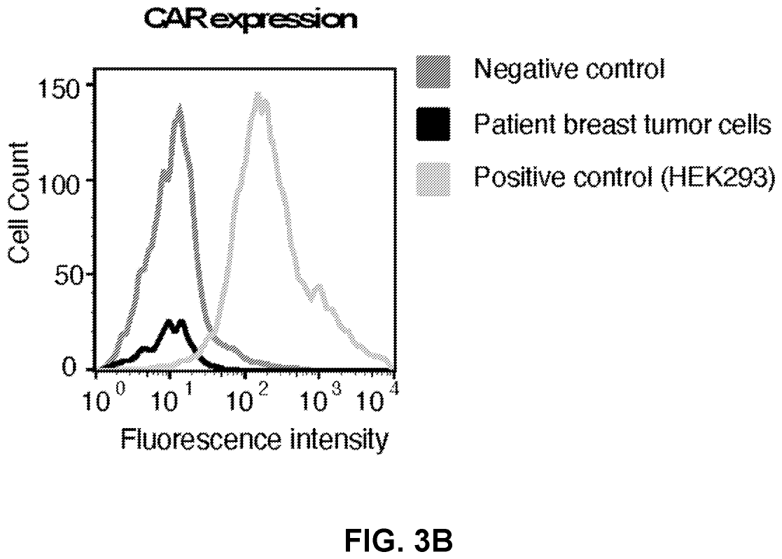

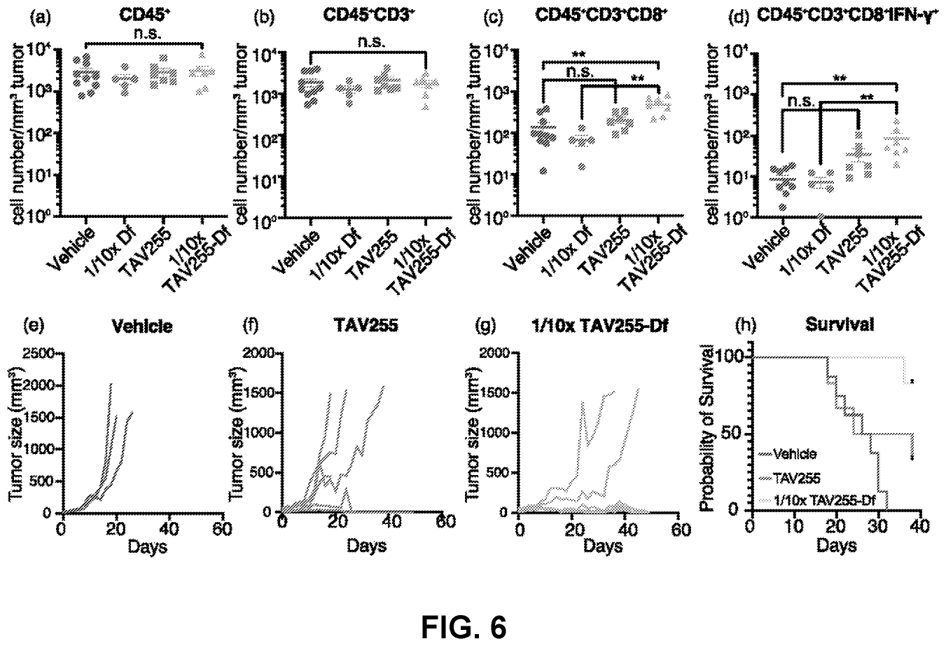

Two studies were performed in more realistic systems. (1) The encapsulated non-replicating Ad was shown to efficiently transfect freshly isolated primary patient breast cancer cells in vitro, which recapitulates the kind of cellular heterogeneity that is likely to be encountered during intratumoral injection and that may impede effective delivery (FIGS. 3A-3B). (2) CT26 tumor-bearing mice treated with encapsulated Ad showed an increase in lymphocytes, an improved survival rate, and complete tumor remission (FIG. 6). The surviving mice also displayed successful immunity in a re-challenge study (FIG. 6). This is believed to be the first example of therapeutic immune memory with a replicating Ad vector in a DOTAP liposome. These results indicate that the encapsulation method of the present disclosure can enhance in vivo adenovirus transfection and result in a better treatment outcome. Thus, the present disclosure provides oncolytic adenovirus formulations to treat aggressive cancers, which frequently express folate receptors at high levels (Meier et al., Radiology, 255 (2): 527-535, 2010).

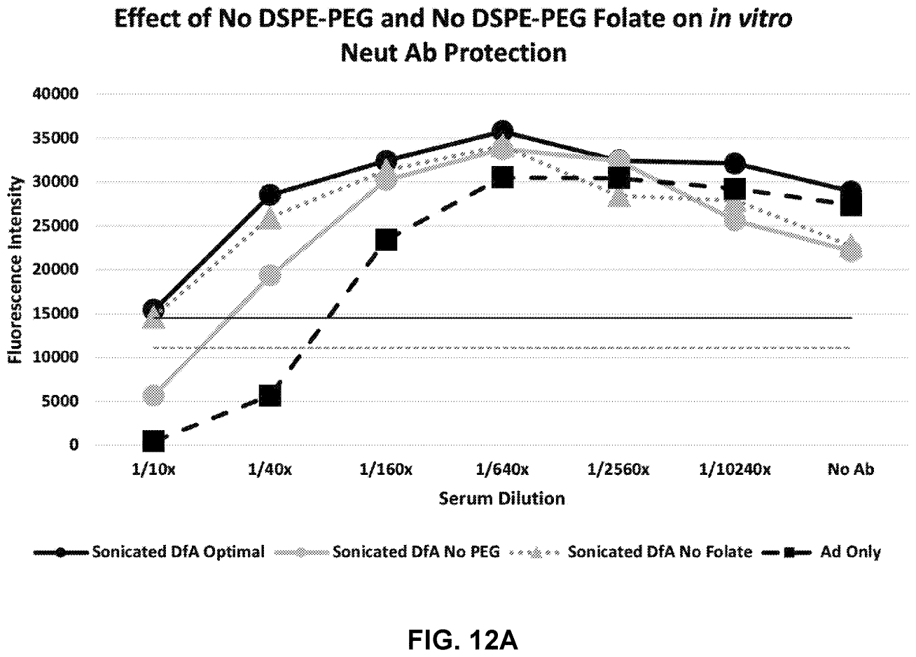

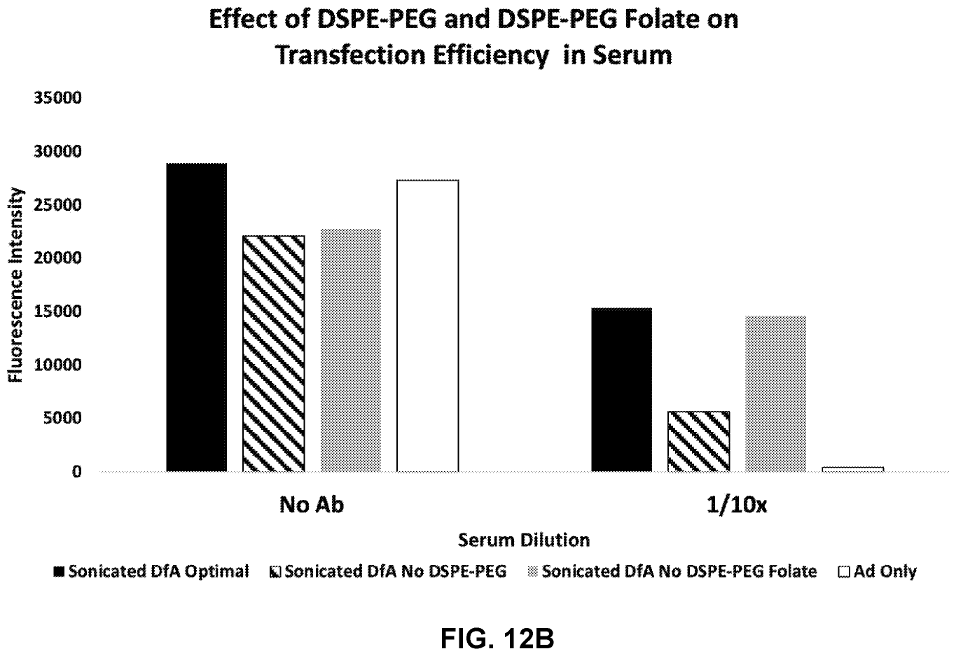

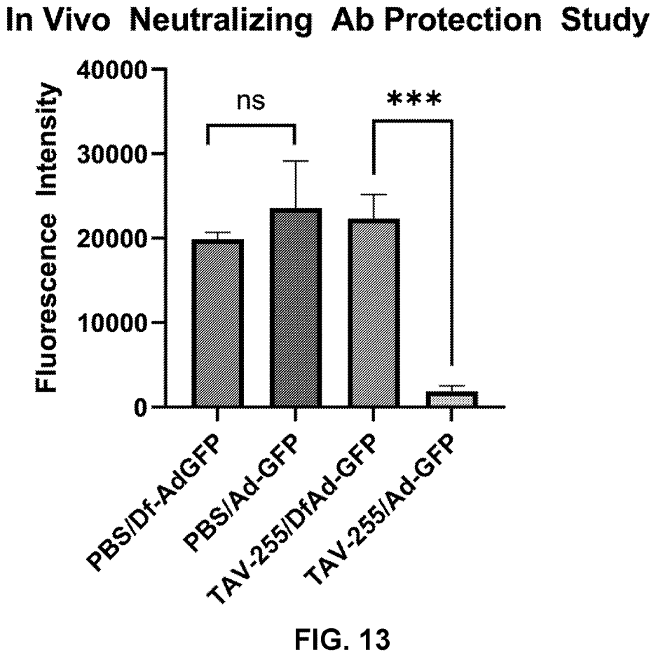

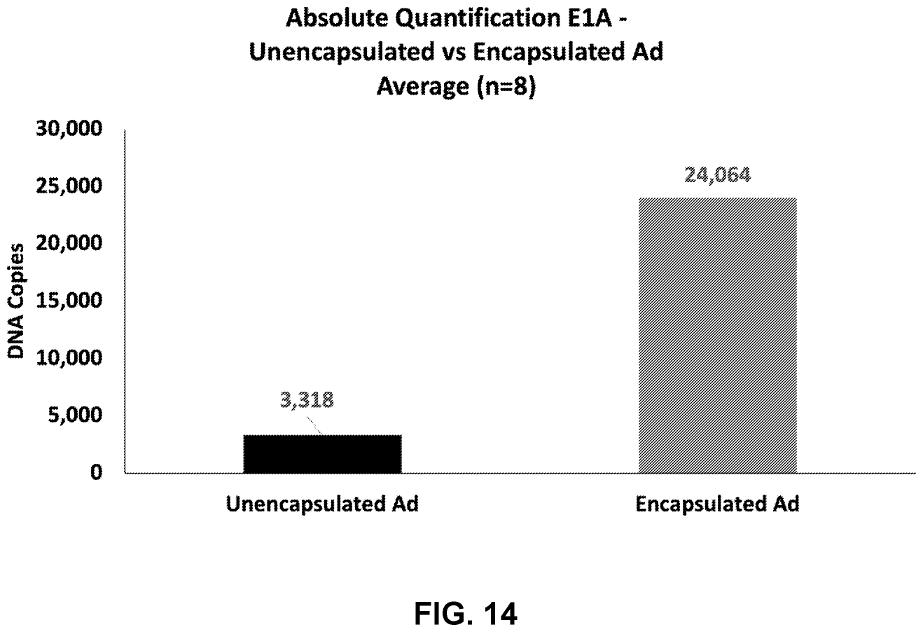

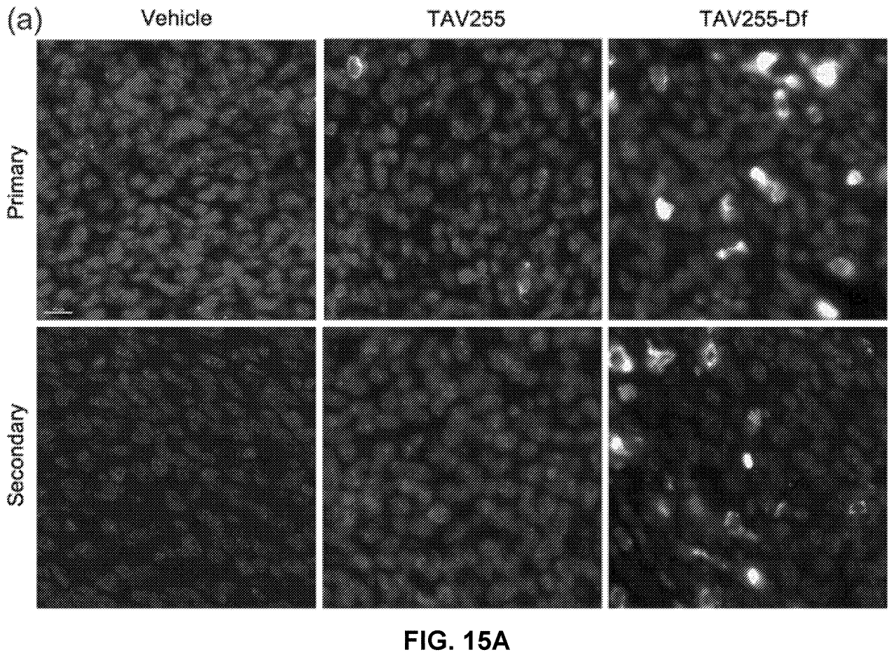

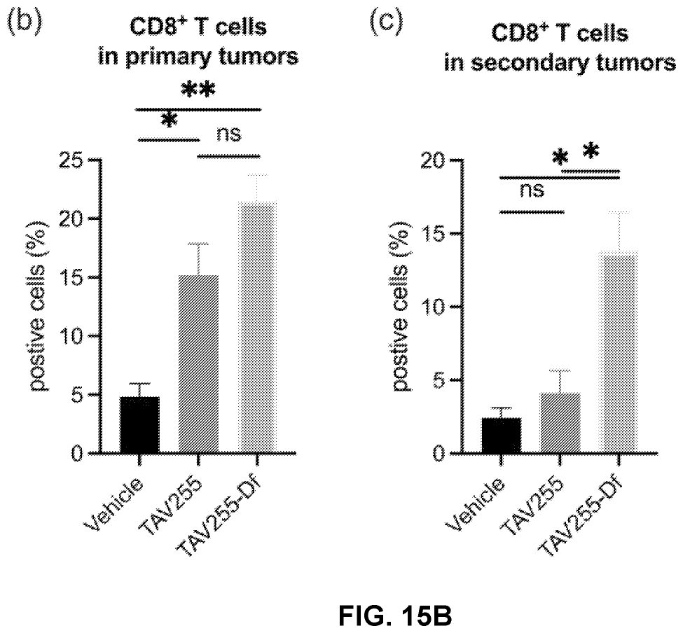

The DOTAP liposome-adenovirus vehicle system of the present disclosure protects the Ad from antibody neutralization and potentially non-specific uptake, which is contemplated to contribute to an extended circulation time and accumulation in metastatic tumors. Furthermore, Ad encapsulation in cationic folate-containing, DOTAP liposomes is contemplated to result in enhanced gene expression in CAR negative cell types. As such, the folate-containing, DOTAP liposome-adenovirus vehicle system is contemplated to be suitable for systemic delivery of adenovirus to metastatic tumor cells. The results demonstrate that encapsulation provides effective protection from neutralizing antibodies and is demonstrated in vitro (FIGS. 12A-12B), facilitates persistence of infectious virus in the systemic circulation in vivo (FIG. 13) and effective virus infection and gene expression in tumors following IV administration (FIG. 14).

The liposomal formulations comprising a folate moiety on the liposome surface have multiple advantageous features including a significantly increased efficacy for epithelial cancer cells. In some embodiments, viral particles encapsulated in folate-bearing DOTAP liposomes can be used to selectively target cancer cells expressing a folate receptor. The targeted cells may include breast cancer cells, pancreatic cells, non-small lung epithelial cancer cells, melanoma cells, sarcoma cells, breast cancer cells, prostate cancer cells, and colorectal cancer cells.

Importantly, the cationic liposome-encapsulated adenovirus formulations of the present disclosure were not found to exhibit the high toxicity and low efficacy expected for a cationic (positively-charged) drug delivery system.

### General Techniques And Definitions

The practice of the present disclosure will employ, unless otherwise indicated, conventional techniques of molecular biology (including recombinant techniques), cell biology, biochemistry, virology, and immunology, which are understood by one of ordinary skill in the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

At various places in the present specification, viruses, compositions, systems, processes, and methods, or features thereof, are disclosed in groups or in ranges. It is specifically intended that the description include each and every individual subcombination of the members of such groups and ranges. By way of example, an integer in the range of 1 to 20 is specifically intended to individually disclose 1, 2, 3, 4, 5, 6, 7, 8, 9, 10, 11, 12, 13, 14, 15, 16, 17, 18, 19 and 20.

The term “about” as used herein in reference to a value, encompasses from 90% to 110% of that value (e.g., a recombinant adenovirus dose of about 20 mg/kg refers to a dose of 18 mg/kg to 22 mg/kg).

The term “plurality” as used herein in reference to an object refers to three or more objects. For instance, “a plurality of adenovirus particles” refers to at least about 10 plaque forming units (pfus), preferably at least about 10² pfus, more preferably from about 10² to about 10¹⁵ pfus.

It should be understood that the order of steps or order for performing certain actions is immaterial so long as the present disclosure remains operable. Moreover, two or more steps or actions may be conducted simultaneously.

The use of any and all examples, or exemplary language herein, for example, “such as” or “including,” is intended merely to illustrate better the present invention and does not pose a limitation on the scope of the disclosure unless claimed. No language in the specification should be construed as indicating any non-claimed element as essential to the practice of the present disclosure.

An “effective amount” or a “sufficient amount” of a substance is that amount sufficient to effect beneficial or desired results, including clinical results, and, as such, an “effective amount” depends upon the context in which it is being applied.

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats).

The term “dose” as used herein in reference to a pharmaceutical formulation refers to a measured portion of the pharmaceutical formulation taken by (administered to or received by) a subject (patient) at any one time.

The terms “isolated” and “purified” as used herein refers to a material that is removed from at least one component with which it is naturally associated (e.g., removed from its original environment). “Isolated” objects are at least 50% free, preferably 75% free, more preferably at least 90% free, and most preferably at least 95% (e.g., 95%, 96%, 97%, 98%, or 99%) free from other components with which they are naturally associated.

As used herein, the term liposome refers to a nano-sized spherical vesicle comprised of an aqueous core that is surrounded by at least one phospholipid bilayer. A “unilamellar vesicle” is a liposome with a single lipid bilayer, while a “multilamellar vesicle” is a liposome with two or more lipid bilayers. In contrast, a “micelle” is a phospholipid aggregate with a monolayer.

The terms “treating” or “treatment” of a disease refer to executing a protocol, which may include administering one or more drugs to an individual (human or otherwise), in an effort to alleviate a sign or symptom of the disease. Thus, “treating” or “treatment” does not require complete alleviation of signs or symptoms, does not require a cure, and specifically includes protocols that have only a palliative effect on the individual. As used herein, and as well-understood in the art, “treatment” is an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival of an individual not receiving treatment. “Palliating” a disease or disorder means that the extent and/or undesirable clinical manifestations of the disease or disorder are lessened and/or time course of progression of the disease or disorder is slowed, as compared to the expected untreated outcome. Further, palliation and treatment do not necessarily occur by administration of one dose, but often occur upon administration of a series of doses.

### I. Preparation of Liposome-Encapsulated Adenovirus Particles

The present disclosure provides multiple methods of preparing liposome-encapsulated adenovirus particles. The liposomes of the present disclosure are cationic liposomes, which comprise 2-dioleoyl-3-trimethylammonium-propane (DOTAP), a pegylated carboxylic acid-terminated phospholipid, and a pegylated folate-containing phospholipid, but not lecithin. In some embodiments, the adenovirus particles are added to pre-formed cationic liposomes. In other embodiments, the cationic liposomes are formed in the presence of adenovirus particles. In some preferred embodiments, the liposomes comprise cholesterol. In some embodiments, formulations comprising cationic lipid-encapsulated adenovirus particles further comprise a serum albumin. In some embodiments, the serum albumin is human serum albumin (HSA), bovine serum albumin, or a rodent serum albumin. In some preferred embodiments, the serum albumin is HSA. In some embodiments, the serum albumin is present in the formulation at a concentration of about 10 mg/mL to 50 mg/mL.

In some embodiments, the present disclosure provides methods of preparing a cationic liposome encapsulating an adenovirus particle, comprising:
a) preparing a lipid mixture by dissolving 1,2-dioleoyl-3-trimethylammonium-propane (DOTAP), a pegylated carboxylic acid-terminated phospholipid, and a pegylated folate-containing phospholipid in an organic solvent;
b) drying the lipid mixture under vacuum to form a dry lipid film;
c) hydrating the dried lipid film by contacting the film with a pharmaceutically acceptable buffer to form a liquid suspension comprising a multilamellar vesicle;
d) physically disrupting the multilamellar vesicle to form an empty cationic liposome; and
e) adding an adenovirus particle to the empty cationic liposome to form a cationic liposome encapsulating the adenovirus particle.

In other embodiments, the present disclosure provides methods of preparing a cationic liposome encapsulating an adenovirus particle, comprising:
a) preparing a lipid mixture by dissolving 1,2-dioleoyl-3-trimethylammonium-propane (DOTAP), a pegylated carboxylic acid-terminated phospholipid, and a pegylated folate-containing phospholipid in an organic solvent;
b) drying the lipid mixture under vacuum to form a dry lipid film;
c) hydrating the dried lipid film by contacting the film with a pharmaceutically acceptable buffer comprising an adenovirus particle to form a liquid suspension comprising a multilamellar vesicle encapsulating the adenovirus particle; and
d) physically-disrupting the multilamellar vesicle to form a cationic liposome encapsulating the adenovirus particle.

In further embodiments, the present disclosure provides methods of preparing a cationic liposome encapsulating an adenovirus particle, comprising:
a) preparing a lipid mixture by dissolving 1,2-dioleoyl-3-trimethylammonium-propane (DOTAP), cholesterol, a pegylated carboxylic acid-terminated phospholipid, and a pegylated folate-containing phospholipid in an organic solvent;
b) drying the lipid mixture under vacuum to form a dry lipid film;
c) hydrating the dried lipid film by contacting the film with a pharmaceutically acceptable buffer to form a liquid suspension comprising a multilamellar vesicle;
d) physically disrupting the multilamellar vesicle to form an empty cationic liposome; and
e) adding an adenovirus particle to the empty cationic liposome to form a cationic liposome encapsulating the adenovirus particle.

Additionally, the present disclosure provides methods of preparing a cationic liposome encapsulating an adenovirus particle, comprising:
a) preparing a lipid mixture by dissolving 1,2-dioleoyl-3-trimethylammonium-propane (DOTAP), cholesterol, a pegylated carboxylic acid-terminated phospholipid, and a pegylated folate-containing phospholipid in an organic solvent;
b) drying the lipid mixture under vacuum to form a dry lipid film;
c) hydrating the dried lipid film by contacting the film with a pharmaceutically acceptable buffer comprising an adenovirus particle to form a liquid suspension comprising a multilamellar vesicle encapsulating the adenovirus particle; and
d) physically-disrupting the multilamellar vesicle to form a cationic liposome encapsulating the adenovirus particle.

In some embodiments, a serum albumin is present in the pharmaceutically acceptable buffer of step c). In some embodiments, the serum albumin is human serum albumin (HSA), bovine serum albumin, or a rodent serum albumin. In some preferred embodiments, the serum albumin is HSA.

Suitable methods for step d), physically disrupting the multilamellar vesicle, include but are not limited to sonicating, extruding, homogenizing, cavitating, and combinations thereof. In some preferred methods, physically-disrupting comprises sonicating the multilamellar vesicle. In some preferred methods, physically disrupting comprises microfluidizing the multilamellar vesicle.

Also provided by the present disclosure are methods of preparing a cationic liposome encapsulating an adenovirus particle:
a) preparing a lipid mixture by dissolving 1,2-dioleoyl-3-trimethylammonium-propane (DOTAP), cholesterol, a pegylated carboxylic acid-terminated phospholipid, and a pegylated folate-containing phospholipid in an organic solvent;
b) microfluidizing the lipid mixture with a pharmaceutically acceptable buffer in a to form a composition comprising an empty cationic liposome in an organic solvent;
c) evaporating the organic solvent from the composition leaving the empty cationic liposome; and
d) adding an adenovirus particle to the empty cationic liposome to prepare a cationic liposome encapsulating the adenovirus particle.

The present disclosure further provides methods of preparing a cationic liposome encapsulating an adenovirus particle comprising:
a) preparing a lipid mixture by dissolving 1,2-dioleoyl-3-trimethylammonium-propane (DOTAP), cholesterol, a pegylated carboxylic acid-terminated phospholipid, and a pegylated folate-containing phospholipid in an organic solvent;
b) microfluidizing the lipid mixture with a pharmaceutically acceptable buffer comprising an adenovirus particle to form a composition comprising a cationic liposome encapsulating the adenovirus particle in an organic solvent;
c) evaporating the organic solvent from the composition leaving the cationic liposome encapsulating the adenovirus particle.

In some embodiments, a serum albumin is present in the pharmaceutically acceptable buffer of step b). In some embodiments, the serum albumin is human serum albumin (HSA), bovine serum albumin, or a rodent serum albumin. In some preferred embodiments, the serum albumin is HSA.

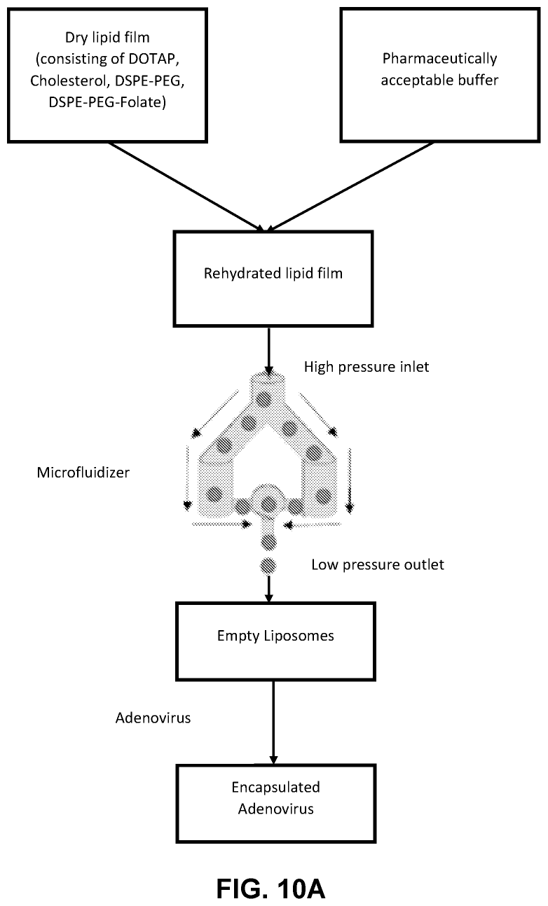

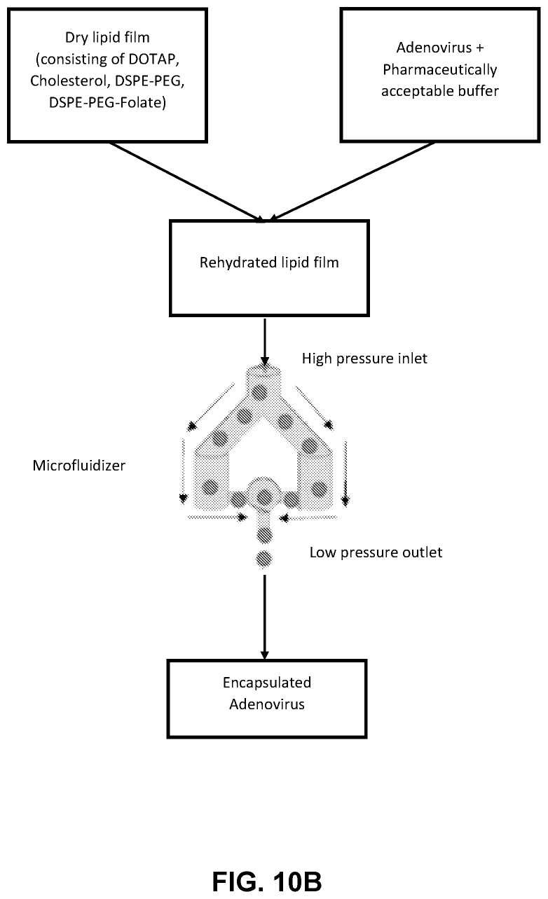

Exemplary methods involving microfluidization are shown in FIGS. 10A-10B. In one microfluidization process, the liquid suspension comprising a multilamellar vesicle formed by hydrating the dry lipid film is divided into two high pressure streams that are passed through a fine orifice before being passed through each other at low pressure in an interaction chamber of a microfluidizer forming an empty cationic liposome to which an adenovirus particle is added to prepare a cationic liposome encapsulating the adenovirus particle (FIG. 10A). In another microfluidization process, the liquid suspension comprising a multilamellar vesicle encapsulated adenovirus particle formed by hydrating the dry lipid film is divided into two high pressure streams that are passed through a fine orifice before being passed through each other at low pressure in an interaction chamber of a microfluidizer to prepare a cationic liposome encapsulating the adenovirus particle (FIG. 10B).

Additional exemplary methods involving microfluidization are shown in FIGS. 10A-10B. In one microfluidization process, the lipid mixture comprising an organic solvent and the pharmaceutically acceptable buffer are placed in separate high pressure streams that are passed through a fine orifice before being passed through each other at low pressure in an interaction chamber of a microfluidizer forming an empty cationic liposome to which an adenovirus particle is added after evaporation of the organic solvent to prepare a cationic liposome encapsulating the adenovirus particle (FIG. 10A). In another microfluidization process, the lipid mixture comprising an organic solvent and the pharmaceutically acceptable buffer comprising an adenovirus particle are placed in separate high-pressure streams that are passed through a fine orifice before being passed through each other at low pressure in an interaction chamber of a microfluidizer to prepare a cationic liposome encapsulating the adenovirus particle after evaporation of the organic solvent (FIG. 10B).

Unless otherwise described, the cationic liposome encapsulating an adenovirus particle prepared according to the methods described herein is a large unilamellar (single lipid bilayer) vesicle encapsulating at least one adenovirus particle, wherein the unilamellar vesicle of the cationic liposome comprises 1,2-dioleoyl-3-trimethylammonium-propane (DOTAP), a pegylated carboxylic acid-terminated phospholipid, and a pegylated folate-containing phospholipid. In some embodiments, the vesicle further comprises cholesterol. In some preferred embodiments, the cationic liposome encapsulating an adenovirus particle is about 100 to about 900 nm in diameter, optionally about 100 to about 700 nm in diameter, optionally about 100 to about 500 nm in diameter, optionally about 100 to about 300 nm in diameter, or about 250 nm in diameter.

Ranges and preferred amounts of ingredients of exemplary cationic liposome-encapsulated adenoviruses are shown in Table I and Table II, with the amount of adenovirus shown as viral particles (vp) per nM DOTAP or as vp/mL, respectively. A description of exemplary methods for producing and testing cationic liposome-encapsulated adenoviruses is provided in Example I.

TABLE I
Formulation Ingredients by Molar Ratio

| | Molar ratio | Molar ratio |
|---|---|---|
| | [Range] | [Optimal] |
| | DOTAP is | DOTAP is |
| Ingredient | the reference | the reference |
| DOTAP | | 1.00 |
| DSPE-PEG-2000 | 0.02-0.04 | 0.02 |
| DSPE-PEG- | 0.01-0.02 | 0.01 |
| 2000-Folate | | |
| cholesterol | 0.00-0.39 | 0.26 |
| HSA | 0.1-50 mg/mL | 20-50 mg/mL |
| Ad (vp/nM) | | ~5.17 × 10⁷ |

TABLE II
Formulation Ingredients by Weight\hat{ }

| Ingredient | [Range] (mg) | [Optimal] (mg) |
|---|---|---|
| DOTAP | | 0.27 |
| DSPE-PEG-2000 | 0.0195-0.039 | 0.0195 |
| DSPE-PEG-2000-Folate | 0.0097-0.194 | 0.0097 |
| cholesterol | 0.00-0.0580 | 0.0387 |
| HSA | 0.1-50 mg/mL | 20-50 mg/mL |
| Ad (vp/mL) | — | ~5.17 × 10¹⁰ |

Since DOTAP is a cationic lipid, the liposomes of the present disclosure are cationic even though their zeta potential is significantly reduced (or made negative) by the incorporation of PEG because of its negative charge. Cationic liposomes are known to bind by electrostatic interactions to the negatively charged surface of tumor cells (Beduneau et al., Biomaterials, 28:4947-4967, 2007). This negative charge is due in part to sialic acid moieties of glycolipids and glycoproteins and to the nearly 200-fold elevation in glycolysis with the associated secretion of lactate anions across the plasma membrane of tumor cells. The secretion of lactate leads to the removal of positive ions from the cell surface, leaving behind negative charges (Ratnam et al., Expert Opin Biol Ther, 3:563-574, 2003).

In some methods of the present disclosure, the amounts of DSPE-PEG-2000 and/or DSPE-PEG-2000-Folate in the liposomes are increased or decreased from the optimal values of Table I and Table II to decrease or increase the zeta potential (ZP) of the liposomes. For instance, when a more positive ZP is desirable, the amounts of DSPE-PEG-2000 and/or DSPE-PEG-2000-Folate are lower than the optimal values (e.g., lower molar ratio to DOTAP than 0.02 and/or 0.01, respectively). In contrast, when a more negative ZP is desirable, the amounts of DSPE-PEG-2000 and/or DSPE-PEG-2000-Folate are higher than the optimal values (e.g., higher molar ratio to DOTAP than 0.02 and/or 0.01, respectively).

As used herein, a positive ZP refers to a ZP measured at pH 7.4 above about +5.0 mv, a negative ZP refers to a ZP measured at pH 7.4 below about −5.0 mv, and a neutral ZP refers to a ZP measured at pH 7.4 of from about −5.0 mv to about +5.0 mv. Thus, when a neutral ZP is desirable, the amounts of DSPE-PEG-2000 and/or DSPE-PEG-2000-Folate are on the order of the optimal values (e.g., molar ratio to DOTAP in the range of ±25%, preferably in the range of ±10% of 0.02 and/or 0.01, respectively). Unless otherwise described, the ZP values are for liposome-encapsulated adenovirus (e.g., not empty liposomes). However, if there is no charge on the surface of the liposomes, then the liposomes can get close enough to start aggregating. Therefore, to formulate stable dispersions of particles, it is helpful for the liposomes to have a small charge, either negative or positive. That is, liposomes with a 0.0 mv ZP are less stable. Hence in some embodiments, the liposomes are formulated to have a ZP below about −0.5 mv and above about +0.5 mv, below about −0.25 mv and above about +0.25 mv, or below about −0.1 mv and above about +0.1.

### II. Adenovirus Particles

A recombinantly modified virus is referred to herein as a “recombinant virus.” A recombinant virus may be modified by recombinant DNA techniques to be replication deficient, conditionally replicating, or replication competent, and/or to express at least one heterologous coding region (e.g., exogenous transgene). In some preferred embodiments of the present disclosure, the adenovirus is a recombinant adenovirus.

Adenoviruses are medium-sized (90-100 nm), non-enveloped (naked), icosahedral viruses composed of a nucleocapsid and a double-stranded linear DNA genome. Adenoviruses replicate in the nucleus of mammalian cells using the host's replication machinery. The term “adenovirus” refers to any virus in the genus Adenoviridiae including, but not limited to, human, bovine, ovine, equine, canine, porcine, murine, and simian adenovirus subgenera. In particular, human adenoviruses include the A-F subgenera, as well as the individual serotypes thereof. The individual serotypes and A-F subgenera including but are not limited to human adenovirus types 1, 2, 3, 4, 4a, 5, 6, 7, 8, 9, 10, 11 (Ad11a and Ad11p), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. Some preferred recombinant adenoviruses are derived from human adenovirus type 2 (Ad2), human adenovirus type 5 (Ad5), or human adenovirus types 35 (Ad35). In some embodiments, the recombinant adenovirus is Ad5. Unless stated otherwise, all Ad5 nucleotide numbers are relative to the NCBI No. AC_000008.1, the nucleotide sequence of which is set forth as SEQ ID NO: 1 and incorporated by reference.

The adenovirus replication cycle has two phases: an early phase, during which four transcription units E1, E2, E3, and E4 are expressed, and a late phase which occurs after the onset of viral DNA synthesis when late transcripts are expressed primarily from the major late promoter (MLP). The late messages encode most of the virus's structural proteins. The gene products of E1, E2 and E4 are responsible for transcriptional activation, cell transformation, viral DNA replication, as well as other viral functions, and are necessary for viral growth.

The E1a gene of Ad5 is processed by mRNA splicing to yield five distinct isoforms; 13S, 12S, 11S, 10S and 9S. The major forms 13S and 12S code for two E1a proteins, 289R and 243R respectively, that regulate transcription of both viral and cellular genes in adenovirus-infected cells and are essential for adenoviral replication. The 289R form includes a critical transactivation domain that activates transcription of the early adenoviral genes: E2, E3, and E4. This domain is spliced out to generate the 243R isoform of E1a and viruses expressing only the 243R form are unable to transactivate expression from the early viral genes. E1a induces expression of cellular genes including c-Fos, c-Jun, and c-Myc and represses the transcription of c-erbB2 and epidermal growth factor receptor. E1a proteins can drive quiescent cells into cell division by interaction with critical cellular cell cycle proteins including pRB, p27, cyclin A, cyclin E, CtBP, and p300/CBP.

The general structure of the mature Adenovirion is conserved among different Adenoviral species. The Adenoviral capsid is composed of three major proteins (II, III, and IV) and five minor proteins, VI, VIII, IX, IIIa, and IVa2. “IVa2 gene” used herein refers to the gene encoding the IVa2 protein, modified versions, and/or fragment thereof. “IX gene” used herein refers to the gene encoding the IX protein, modified versions, and/or fragment thereof.

Primary transcripts from E4 are subject to alternative splicing events and are predicted to encode seven different polypeptides: ORF1, ORF2, ORF3, ORF3/4, ORF4, ORF5, ORF6, and ORF6/7. “ORF” is used herein to refer to either the polypeptide or the nucleotide sequence encoding the polypeptide, modified versions, and/or fragment thereof.

In addition, the fiber protein (also known as protein IV or SPIKE) forms spikes that protrude from each vertex of the icosahedral capsid. “Fiber gene” used herein refers to the gene encoding the fiber protein, also known as L5 gene, modified versions, and/or fragment thereof.

Recombinant adenoviruses and methods of making and using them are described in U.S. Pat. Nos. 9,073,980, 10,876,097 and 11,253,608, as well as Publication Nos. US 2019/0352669, US 2019/0352616, US 2020/0032223, US 2020/0078415 and US 2021/0015878. The details of the modified transcriptional control regions and transgene insertion sites of the foregoing U.S. patents and published applications are incorporated by reference.

### A. Modified Transcriptional Control Region

In certain embodiments, the recombinant adenoviruses comprise one or more modifications to a regulatory sequence or promoter. A modification to a regulatory sequence or promoter comprises a deletion, substitution, or addition of one or more nucleotides compared to the wild-type sequence of the regulatory sequence or promoter.

In one embodiment, the modification of a regulatory sequence or promoter comprises a modification of sequence of a transcription factor binding site to reduce affinity for the transcription factor, for example, by deleting a portion thereof, or by inserting a single point mutation into the binding site. In certain embodiments, the additional modified regulatory sequence enhances expression in neoplastic cells but attenuates expression in normal cells.

The E1a regulatory sequence contains five binding sites for the transcription factor Pea3, designated Pea3 I, Pea3 II, Pea3 III, Pea3 IV, and Pea3 V, where Pea3 I is the Pea3 binding site most proximal to the E1a start site, and Pea3 V is most distal. The E1a regulatory sequence also contains binding sites for the transcription factor E2F, hereby designated E2F I and E2F II, where E2F I is the E2F binding site most proximal to the E1a start site, and E2F II is more distal. From the E1a start site, the binding sites are arranged: Pea3 I, E2F I, Pea3 II, E2F II, Pea3 III, Pea3 IV, and Pea3 V. In some embodiments, at least one of these seven binding sites is deleted or otherwise rendered non-functional such that it is less capable of binding a respective binding partner (e.g., a binding site that less than 30%, less than 20%, less than 10%, or 0% of the binding activity of a corresponding wild-type binding site sequence).

Previously developed oncolytic viruses include the oncolytic serotype 5 adenovirus (Ad5) referred to as TAV-255 in U.S. Pat. Nos. 9,073,980 and 10,876,097. TAV-255 is transcriptionally attenuated in normal cells but transcriptionally active in cancer cells. It is believed that the mechanism by which the TAV-255 vector achieves this tumor selectivity is through targeted deletion of three transcriptional factor binding sites for the transcription factors Pea3 and E2F, proteins that regulate adenovirus expression of E1a, the earliest gene to be transcribed after virus entry into the host cell, through binding to specific DNA sequences. These three Pea3 and E2F deletions attenuate replication in growth-arrested, normal cells but not in malignant ones, indicating that these DNA sequences are only dispensable for transcriptional regulation and growth in cancer cells.

In certain embodiments, any of the foregoing recombinant adenoviruses comprises a modified E1a regulatory sequence. In certain embodiments, the recombinant adenovirus comprises an E1a promoter having a deletion of a functional Pea3 binding site. For example, the virus may comprise a deletion of nucleotides corresponding to about −300 to about −250 upstream of the initiation site of E1a or a deletion of nucleotides corresponding to −305 to −255 upstream of the initiation site of E1a. In certain embodiments, the deletion comprises a deletion of nucleotides corresponding to 195-244 of the Ad5 genome (SEQ ID NO:1). In some embodiments, the modified E1a promoter comprises the sequence GGTGTTTTGG (SEQ ID NO:2).

### B. Insertion Sites

In certain embodiments, the recombinant adenovirus comprises one or more nucleotide sequences comprising a transgene inserted in one of more of an E1b-19K insertion site, an E3 insertion site, an E4 insertion site, an IX-E2 insertion site, an L5-E4 insertion site, and combinations thereof.

In certain embodiments, the E1b-19K insertion site is located between the start site of E1b-19K and the start site of E1b-55K. The adenoviral E1b-19k gene functions primarily as an anti-apoptotic gene and is a homolog of the cellular anti-apoptotic gene, BCL-2. Since host cell death prior to maturation of the progeny viral particles would restrict viral replication, E1b-19k is expressed as part of the E1 cassette to prevent premature cell death thereby allowing the infection to proceed and yield mature virions. Accordingly, in certain embodiments, a recombinant virus is provided that includes an E1b-19K insertion site, e.g., the adenovirus has an exogenous nucleotide sequence inserted into an E1b-19K insertion site. In certain embodiments, the insertion site is located between the start site of E1b-19K and the stop codon of E1b-19K.

In certain embodiments, the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent to the start site of E1b-19K. In certain embodiments, the E1b-19K insertion site comprises a deletion of about 200 nucleotides, e.g., 202 nucleotides adjacent to the start site of E1b-19K. In certain embodiments, the E1b-19K insertion site comprises a deletion corresponding to nucleotides 1714-1917 of the Ad5 genome (SEQ ID NO:1), or an exogenous nucleotide sequence encoding a transgene is inserted between nucleotides corresponding to 1714 and 1917 of the Ad5 genome (SEQ ID NO:1). In certain embodiments, an exogenous nucleotide sequence encoding a transgene is inserted between CTGACCTC and TCACCAGG, e.g., the recombinant adenovirus comprises, in a 5′ to 3′ orientation, CTGACCTC, an exogenous nucleotide sequence encoding a transgene, and TCACCAGG.

In certain embodiments, the E3 insertion site is located between the stop codon of pVIII and the start site of Fiber. In certain embodiments, the E3 insertion site is located between the stop codon of E3-10.5K and the stop codon of E3-14.7K and the start site of Fiber.

In certain embodiments, the E3 insertion site comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or about 3000 to about 3185 nucleotides. In certain embodiments, the E3 insertion site is located between the stop codon of E3-10.5K and the stop codon of E3-14.7K. In certain embodiments, the E3 insertion site comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop codon of E3-10.5K. In certain embodiments, the E3 insertion site comprises a deletion of about 1050 nucleotides adjacent the stop codon of E3-10.5K, e.g., the E3 insertion site comprises a deletion of 1063 nucleotides adjacent the stop codon of E3-10.5K. In certain embodiments, the E3 insertion site comprises a deletion corresponding to the Ad5 dl309 E3 deletion. In certain embodiments, the E3 insertion site comprises a deletion corresponding to nucleotides 29773-30836 of the Ad5 genome (SEQ ID NO:1).

In certain embodiments, an E4 insertion site comprises any one of the ORF of the E4 gene. For example, a nucleotide sequence can be inserted in E4 ORF1, and/or E4 ORF2. In certain embodiments, portions of or the entire E4 region may be deleted.

In certain embodiments, the insertion site is the IX-E2 insertion site. In certain embodiments, the IX-E2 insertion site is located between the stop codon of adenovirus IX gene and the stop codon of adenovirus IVa2 gene. In certain embodiments, the nucleotide sequence is inserted between nucleotides corresponding to 4029 and 4093 of the Ad5 genome (SEQ ID NO:1). In certain embodiments, the nucleotide sequence is inserted between nucleotides corresponding to 4029 and 4050, nucleotides corresponding to 4051 and 4070, or nucleotides corresponding to 4071 and 4093 of the Ad5 genome (SEQ ID NO:1). In some embodiments, the IX-E2 insertion site has at least 60%, at least 70%, at least 80%, at least 90%, at least 95%, or at least 99% identity to nucleotides corresponding to 4029 and 4093 of the Ad5 genome (SEQ ID NO:1).

In certain embodiments, the insertion site is an L5-E4 insertion site. In certain embodiments, the L5-E4 insertion site is located between the stop codon of adenovirus fiber gene and the stop codon of ORF6 or ORF6/7 of the adenovirus E4 gene. In certain embodiments, the nucleotide sequence is inserted between nucleotides corresponding to 32785 to 32916 of the Ad5 genome (SEQ ID NO:1). In certain embodiments, the nucleotide sequence is inserted between nucleotides corresponding to 32785 and 32800, nucleotides corresponding to 32801 and 32820, nucleotides corresponding to 32821 and 32840, nucleotides corresponding to 32841 and 32860, nucleotides corresponding to 32861 and 32880, nucleotides corresponding to 32881 and 32900, or nucleotides corresponding to 32901 and 32916 of the Ad5 genome (SEQ ID NO: 1). In some embodiments, the L5-E4 insertion site has at least 60%, at least 70%, at least 80%, at least 90%, at least 95%, or at least 99% identity to nucleotides corresponding to 32785 to 32916 of the Ad5 genome (SEQ ID NO: 1).

In certain embodiments, the IX-E2 insertion site comprises a deletion of about 5, 10, 15, 20, 25, 30, 35, 40, 45, 50, 55, or 60 nucleotides. In certain embodiments, the L5-E4 insertion site comprises a deletion of about 5, 10, 15, 20, 25, 30, 35, 40, 45, 50, 55, 60, 65, 70, 75, 80, 85, 90, 95, 100, 105, 110, 115, 120, 125, or 130 nucleotides.

In certain embodiments, the recombinant adenovirus comprises two or more nucleotide sequences, wherein the nucleotide sequences each comprises a transgene, wherein the nucleotide sequences are optionally separated by a linker. In certain embodiments, the recombinant adenovirus expresses two transgenes, when expressed, produce a single polypeptide chain, which may be cleaved post-translationally into two polypeptide chains. In certain embodiments, the linker is an internal ribosome entry site (IRES) element and/or a self-cleaving 2A peptide sequence. The IRES may, e.g., be selected from the group consisting of the encephalomyocarditis virus IRES, the foot-and-mouth disease virus IRES, and the poliovirus IRES.

In certain embodiments, the two or more nucleotide sequences are inserted in an E1b-19K insertion site, an E3 insertion site, an E4 insertion site, an IX-E2 insertion site, or an L5-E4 insertion site. In certain embodiments, the two or more nucleotide sequences are inserted in the same insertion site. In certain embodiments, the two or more nucleotide sequences are inserted in different insertion sites.

### III. Methods for Virus Production

Nucleic acids encoding coronavirus antigens can be incorporated into plasmids and introduced into host cells through conventional transfection or transformation techniques. Specific production and purification conditions will vary depending upon the virus and the production system employed. For adenovirus, the traditional method for the generation of viral particles is co-transfection followed by subsequent in vivo recombination of a shuttle plasmid (usually containing a small subset of the adenoviral genome and optionally containing a potential transgene an expression cassette) and an adenoviral helper plasmid (containing most of the entire adenoviral genome).

### VI. Pharmaceutical Compositions

For prophylactic or therapeutic use, a recombinant adenovirus disclosed herein is preferably combined with a pharmaceutically acceptable excipient. As used herein, prophylactic and therapeutic uses include preclinical and clinical uses. Pharmaceutically acceptable excipients of the present disclosure include for instance, solvents, bulking agents, buffering agents, tonicity adjusting agents, and preservatives (see, e.g., Pramanick et al., Pharma Times, 45:65-77, 2013). In some embodiments the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). As used herein, “pharmaceutically acceptable” means the excipient is suitable for use in contact with the tissues of humans and other mammalian subjects without excessive toxicity, irritation, allergic response, or other problem or complication, commensurate with a reasonable benefit/risk ratio. The excipient should be “acceptable” in the sense of being compatible with other ingredients of the formulation and not deleterious to the recipient.

Pharmaceutical compositions can be provided in a dosage unit form and can be prepared by any suitable method. A pharmaceutical composition should be formulated to be compatible with its intended route of administration. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method (e.g., filtration through sterile filtration membranes). Where the composition is lyophilized, filter sterilization can be conducted prior to or following lyophilization and reconstitution.

The term “effective amount” as used herein refers to the amount of an active component (e.g., recombinant human adenovirus) sufficient to effect beneficial or desired results. An effective amount can be administered in one or more administrations, applications or dosages and is not intended to be limited to a particular formulation or administration route.

In certain embodiments, an effective amount of active agent is in the range of 0.1 mg/kg to 100 mg/kg, preferably 0.5 mg/kg to 20 mg/kg, or preferably 1 mg/kg to 10 mg/kg. The amount administered will depend on variables such as the type and extent of disease or indication to be treated or prevented, the overall health of the patient, the in vivo potency of the active agent, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized for instance in a conventional Phase I dose escalation study. Dosing frequency can vary, depending on factors such as route of administration, dosage amount, and serum half-life of the active agent. In certain embodiments, an effective amount of a recombinant adenovirus is in the range of 10² to 10¹⁵ plaque forming units (pfus) (e.g., 10² to 10¹⁰, 10² to 10⁵, 10⁵ to 10¹⁵, 10⁵ to 10¹⁰, or 10¹⁰ to 10¹⁵ pfus).

### V. Methods of Use

### A. Treatment

The present disclosure relates to methods for use of pharmaceutical formulations comprising an adenovirus particle encapsulated in a cationic liposome including a cancer cell-targeting moiety. In particular, the present disclosure relates to use of pharmaceutical formulations comprising a folate-containing, cationic liposome-encapsulated adenovirus particle for treating cancer in a mammalian subject.

The present disclosure relates to methods for treating cancer in a human subject comprising administering an effective amount of a pharmaceutical formulation of the folate-containing, cationic liposome-encapsulated adenovirus particle as described herein to a human subject to treat the cancer. “Treating” cancer means to bring about a beneficial clinical result such as causing remission or otherwise prolonging survival as compared to expected survival in the absence of treatment. In some embodiments, “treating” cancer comprises shrinking the size of a tumor or otherwise reducing viable cancer cell numbers. In other embodiments, “treating” cancer comprises delaying growth of a tumor. In some embodiments, the formulation is administered by intra-tumoral or peri-tumoral delivery. In some embodiments, the formulation is administered by parenteral delivery. In some embodiments, the formulation is administered by intradonal delivery. In some embodiments, the formulation is administered by intravenous, intraarterial or subcutaneous injection. In some embodiments, the cancer is a carcinoma. In some embodiments, the cancer is an adenocarcinoma or a squamous cell carcinoma. In some embodiments, the cells of the cancer are coxsackievirus-adenovirus receptor (CAR) negative. In some embodiments, the cells of the cancer are coxsackievirus-adenovirus receptor (CAR) positive. In some embodiments, the cells of the cancer are folate receptor positive. In some embodiments, the cells of the cancer are folate receptor negative.

In some embodiments, the zeta potential (ZP) of liposome is tailored to the mode of administration and the type of tumor to be treated. For instance, in some embodiments, a folate-containing, cationic liposome-encapsulated adenovirus particle with a positive ZP is administered intratumorally or peritumorally to a highly glycolytic tumor having a standard uptake value on PET scan of >10. In other embodiments, a folate-containing, cationic liposome-encapsulated adenovirus particle with a neutral ZP is administered intratumorally or peritumorally to a tumor with low glycolysis having a standard uptake value on PET scan of <10. In some embodiments, a folate-containing, cationic liposome-encapsulated adenovirus particle with a neutral ZP is administered intravenously. In other embodiments, a folate-containing, cationic liposome-encapsulated adenovirus particle with a negative ZP is administered intravenously.

### B. Evaluation

The present disclosure further relates to use of pharmaceutical formulations comprising a folate-containing, cationic liposome-encapsulated adenovirus particle for evaluating cancer in a mammalian subject. In particular, the present disclosure relates to use of pharmaceutical formulations comprising a folate-containing, cationic liposome-encapsulated adenovirus particle for imaging cancer in a mammalian subject

The present disclosure related to methods of determining margins of a tumor in a surgical patient, the method comprising: a) administering an effective mount of a pharmaceutical formulation of the folate-containing, cationic liposome-encapsulated adenovirus particle as described here into the surgical patient; b) allowing time for the adenovirus particles to enter into and replicate within cells of the tumor thereby producing a plurality of adenovirus progeny; and c) detecting the adenovirus progeny thereby determining margins of the tumor, wherein the adenovirus particles express a detectable protein when replicating within cells of the tumor.

### EXAMPLES

Abbreviations: Ad (adenovirus); CAR (coxsackievirus-adenovirus receptor); CMV (cytomegalovirus); Df (DOTAP-folate liposome); DOTAP (1,2-dioleoyl-3-trimethylammonium-propane); FR (folate receptor); GFP (green fluorescent protein); HSA (human serum albumin); MOI (multiplicity of infection); PEG (polyethylene glycol); DSPE-PEG-carboxylic acid (1,2-distearoyl-sn-glycero-3-phosphoethanolamine-N-[carboxy (PEG)-2000); and DSPE-PEG-folate (1,2-distearoyl-sn-glycero-3-phosphoethanolamine-N-[folate (PEG)-2000); and pfu (plague forming unit).

### Example 1. Remission of CAR-Deficient Tumors by Intratumoral Injection of DOTAP-Folate Liposome Encapsulation of Adenovirus

### Materials and Methods

Reagents and Cell Lines. Replication-deficient Ad expressing GFP (GFPAd) was purchased from Baylor College of Medicine (Vector: Ad5-CMV-eGFP). The CT26 cell line was purchased from American Type Culture Collection (ATCC). Replication-competent Ad (TAV255), HEK293, A549, MCF7, and THP-1 cell lines were generously provided. Dulbecco's Modified Eagle Medium (DMEM) with high glucose (HyClone #SH30081.01) was supplemented with 10% of Fetal Bovine Serum (FBS, Corningt #35-011-CV) and 1% of Pen Strep Glutamine (PSG, Life Technologies #10378-016) to prepare the complete media for HEK293, A549, and MCF7 cell culturing. Rosewell Park Memorial Institute (RPMI) 1640 (Gibco #11875093) and RPMI 1640 medium no folic acid (Gibco #27016021) were supplemented with 10% FBS and 1% of PSG to prepare the complete RPMI (RP-10) for CT26 and THP-1 cell culturing. Primary human breast cell medium was prepared by supplementing DMEM/F12 (1:1) with HEPES (HyClone #SH30023.01) with 10 mM HEPES (Sigma #H3537), 5% FBS, 1 mg/mL bovine serum albumin (BSA, Sigma #A7906), 1 μg mL⁻¹ insulin (Invitrogen #51500-056), 0.5 μg mL⁻¹ hydrocortisone (Sigma #H0888), 50 μg mL⁻¹ gentamycin (HyClone #3V30080.01), and 2.5 μg mL⁻¹ Fungizone. Human tumor digestion buffer was prepared with DMEM/F12+GlutaMAX (Gibco #10565018) supplemented with 10 mM HEPES, 2% BSA, 5 μg mL⁻¹ insulin, 0.5 μg mL⁻¹ hydrocortisone, and 50 μg mL⁻¹ gentamycin. Anti-CAR antibody (clone RmcB, #05-644) was purchased from Millipore, and Alexa Fluor 547 conjugated antibody (polyclonal, #A-21235) was purchased from Invitrogen.

Liposome encapsulated adenovirus synthesis. 1,2-dioleoyl-3-trimethylammonium-propane (DOTAP, Avanti #890890C), cholesterol (Sigma #C3045), 1,2-distearoyl-sn-glycero-3-phosphoethanolamine-N-[carboxy (polyethylene glycol)-2000] (PEG (2000)-PE carboxylic acid, Avanti #880135P), and 1,2-distearoyl-sn-glycero-3-phosphoethanolamine-N-[folate (polyethylene glycol)-2000] (PEG (2000)-folate-PE, Avanti #880124P) were suspended in chloroform at molar ratio 1:0.26:0.02:0.01. The lipid mixture was vortexed in an amber vial for 30 minutes at 25° C. The mixture was vacuumed overnight to form a dry lipid film. The next day, 5×10¹⁰ vp/mL of adenovirus (Ad) solution was prepared in PBS containing various concentrations (0 mg/mL, 0.1 mg/mL, 1.0 mg/mL, 20.0 mg/mL, 30.0 mg/mL, or 50.0 mg/mL) of human serum albumin (HSA). The dried lipid film was hydrated with 400 μL of Ad solution while vortexing. The hydrated film was stirred at 600 rpm at 4° C. for 30 minutes. The sample was transferred to a 2 mL U-bottomed Eppendorf tube (Eppendorf North America, #022363352) and sonicated in an ultrasonic water bath (Fisher Scientific, Model FS11011) for 10 minutes at 4° C. The suspension was stabilized at 4° C. for 3 hours resulting in DOTAP-folate Ad liposomes (Ad-Df). Replication-deficient Ad (GFPAd) and replication-competent Ad (TAV255) were used for Ad-Df synthesis and denoted as GFPAd-Df and TAV255-Df, respectively. 1/1×, ⅓×, ⅕×, 1/7×, and 1/10× Ad-Df represent the Ad to DOTAP lipid ratio (vp:nmol) 5.17×10⁶, 1.55×10⁷, 2.59×10⁷, 3.62×10⁷, and 5.17×10⁷, respectively.

Measurement and importance of zeta potential. The zeta potential of liposomes approximates their overall charge in a particular medium, the knowledge of which can help to predict the fate of the liposomes in vivo. A large positive or negative zeta potential of liposomes (e.g., −/+30 mV) is a measure of good physical stability due to electrostatic repulsion. By contrast, at small zeta potential values repulsive forces are diminished and van der Waals attractive forces predominate so that aggregation and flocculation may occur. In brief, measurement of the zeta potential is an electrophoresis-based process in which a voltage is applied across a pair of electrodes at either end of a cell containing the liposome sample in solution. Liposomes with a net charge or zeta potential will migrate to the oppositely charged electrode with a velocity, known as mobility, from which the zeta potential is calculated using an apparatus known as the Zetasizer Nano ZS90.

Transfection. Cells were plated overnight at 3×10⁴ cells well⁻¹ in a 96-well plate at 37° C. and 5% CO₂ in the complete media. GFPAd, 1/1×, ⅓×, ⅕×, 1/7×, and 1/10× GFPAd-Df were added to cells (day 1) at a MOI ranging from 3.1 to 400 and incubated at 37° C. and 5% CO₂. GFP fluorescence was measured on day 2, 3, 4, 5, and 6 for HEK293, A549, MCF7, THP-1 and CT26, respectively. GFP positive cells were counted microscopically using a Keyence BZ-X710 microscope with a GFP filter and 470/40 nm excitation wavelength, 525/50 nm emission wavelength and dichroic mirror wavelength 495 nm. GFP fluorescence intensities were measured using a Tecan Infinite M200 microplate reader.

Human breast cells isolation and cell culture. All human breast cancer biospecimens were obtained from patients. Patient consent was obtained by BTTSR and all procedures were conducted under an Institutional Review Board-approved protocol. Tumor fragments were acquired from two different areas of the same tumor when possible. During the transportation, the acquired tumor tissues were placed in a 50 mL conical tube with sterile PBS such that the tissue sample was entirely submerged in the PBS. 2 mg mL⁻¹ of type 3 collagenase (Worthing #LS004182) and hyaluronidase (Sigma #H3884) 100 U mL⁻¹ in human tumor digest buffer were prepared. Per gram of the tissue, 10 mL of digestion buffer containing enzymes was added into a well in 6-well plate. Tissue was placed into the well and minced until finely chopped. If needed, then a syringe plunger was used to smash the tumor. Resultant tissue was incubated at 37° C. and 5% CO₂ with pipette mixing performed every 30 minutes. After 5 hours of incubation tissue was strained using a 100 μm strainer and the filtrate was centrifuged at 530×g at room temperature for 5 minutes to collect cells. If red blood cells were observed, then 5-10 mL of ACK buffer (Quality Biological #118-156-101) was added and incubated for 3 minutes. Cells were centrifuged at 530×g at 25° C. for 5 minutes and the supernatant was removed. This step was repeated until the red blood cells were no longer visible. Resultant cells were resuspended in 10 mL of primary human breast cell medium, and aliquot of 10 μL was used for cell counting. Cells were plated at minimal 10,000 cells well-1 in a 96-well plate. Viral infection was performed after cells attached to the well (24-48 hours after plating).

Hexon staining. Cells were plated in a 96 well plate at approximately 3×10⁴ cells well⁻¹ in 100 μL media/well. The next day, cells were transfected with TAV255, 1/1×TAV255-Df, ⅓×TAV255-Df, ⅕×TAV255-Df, 1/7×TAV255-Df and 1/10×TAV255-Df (day 1) at a MOI ranging from 3.1 to 400 at 37° C. and 5% CO₂. At the end of transfection, media was gently pipetted off and 50 μL well⁻¹ of cold methanol was added and incubated at −20° C. for 20 minutes. Wells were washed once with 100 μL well⁻¹ of PBS-T (Teknova #P3189, diluted to 1× in PBS). 100 μL well⁻¹ of StartingBlock (Thermo #37542) was added to block the cells for 30 minutes at room temperature. 50 μL well⁻¹ of diluted anti-hexon (Thermo #MA1-7326, diluted 1:500 in StartingBlock) was added and incubated for 1 hour at room temperature. After washes with 100 μL well⁻¹ of PBS-T, 50 μL well⁻¹ of HRP anti-mouse (Cell Signaling #7076, diluted 1:500 in StartingBlock) was added and incubated for 30 minutes at room temperature. DAB was prepared by mixing 3 μL of concentrate per 100 μL diluent from the kit (Cell Signaling #8059P). Plate was washed with 100 μL well⁻¹ of PBS-T twice, and 40 μL well⁻¹ of DAB substrate was added into the wells. After the cells were sufficiently stained, as observed under a microscope, the DAB was discarded and 100 μL well⁻¹ of PBS was added. Hexon-positive cells were imaged and counted under a microscope.

Cell viability assay. Cells were seeded in a 96-well plate at approximately 3×10⁴ cells well⁻¹ in 100 μL media well⁻¹. The next day, TAV255, ⅓×TAV255-Df, and 1/10×TAV255-Df were added to cells (day 1) at a MOI ranging from 3.1 to 400 and incubated at 37° C. and 5% CO₂. On day 2, 4, and 6 post-infection, 1×10⁴ cells were harvested and incubated with Alamar Blue for 1-4 hours. The cell viability was determined by measuring absorbance at wavelengths of 570 nm and 600 nm.

Animals. Six to eight-week-old female BALB/cAnNHsd mice were purchased from Envigo RMS, LLC.

In Study 1, 5×10⁵ CT26 (colon cancer) cells in 50 μL PBS were subcutaneously injected. Tumor volume was determined by caliper with the modified ellipsoidal formula: volume (mm³)=(width×width×length)/2 and treatment was started at a tumor size of approximately 23 mm³. 5×10⁹ vp of TAV255 or 1/10×TAV255-Df, or equivalent 1/10×Df, or PBS were intratumorally injected every other day.

In Study 2, 1×10⁶ of CT26 (colon cancer) cells in PBS were subcutaneously injected. Tumor volume was determined by caliper with the formula: volume (mm³)=(width×width×length)/2 and treatment was started at a tumor size of approximately 23 mm³. Tumor growth was monitored with a caliper until mice reached pre-defined humane or experimental end points. Tumor volume (mm3) was calculated by the formula: length×width×width×0.52). Mice were randomly divided into three groups: 1) mice in the control group were treated with PBS; 2) mice in the TAV group were treated with naked adenovirus (5×10⁹ vp TAV255); and 3) mice in the DfTAV group were treated with adenovirus encapsulated in folate containing liposomes (5×10⁹ vp TAV255-Df). Surviving mice from the TAV and DfTAV groups (n=6) were re-challenged with 1×10⁶ CT26 cells on the opposite flank two weeks after termination of treatment. As a control, naïve age-matched mice (n=5) were implanted with 1×10⁶ CT26 cells. Subsequently, the CT26-experienced mice (n=6) were re-challenged four weeks later with 1×10⁵ 4T1 cells. Tumor growth was monitored, and the number of tumor free mice are shown in the adjacent table. As a control, naïve age-matched mice (n=4) were implanted with 1×10⁵ 4T1 cells.

In both studies, pain and distress in tumor-bearing mice were closely monitored. For the survival study, mice were euthanized when a tumor ulcerated or reached 1,500 mm³. All procedures and protocols were approved by an Institutional Animal Care and Use Committee.

CAR analysis. Cells were incubated with anti-CAR antibody at 1:500 dilution in BD staining buffer at 4° C. for 30 minutes. Cells were washed once with BD staining buffer and incubated with secondary Alexa Fluor 647 conjugated antibody at 1:400 dilution in BD staining buffer for 30 minutes at 4° C. The stained cells were washed twice with BD staining buffer and resuspended in 200 μL PBS. The stained cells were analyzed by BD FACSCalibur flow cytometry.

Tumor infiltrating lymphocytes analysis. CT26-bearing mice was first treated at a tumor size of approximately 70 mm³. A 2.5×10⁹ vp of TAV255 or 1/10×TAV255-Df in 50 μL PBS were injected i.t. on day 0, 2, 4, 6, 8, 10. Mice were sacrificed on day 12 for TIL analysis. Tumors were dissociated into cell suspensions using collagenase D (Roche #11088866001) at 1 mg mL⁻¹ in RP10. Cell suspensions were incubated with cocktails of anti-mouse CD45 (BioLegend #103114), anti-mouse CD3 (BD Biosciences #553062) and anti-mouse CD8 (eBiosciences #48-0081) antibodies at 4° C. for 30 minutes. Fixation and Permeabilization Solution kits were used for IFN-γ staining (eBiosciences #17-7311). The stained cells were analyzed by BD FACSCanto flow cytometry.

### Results

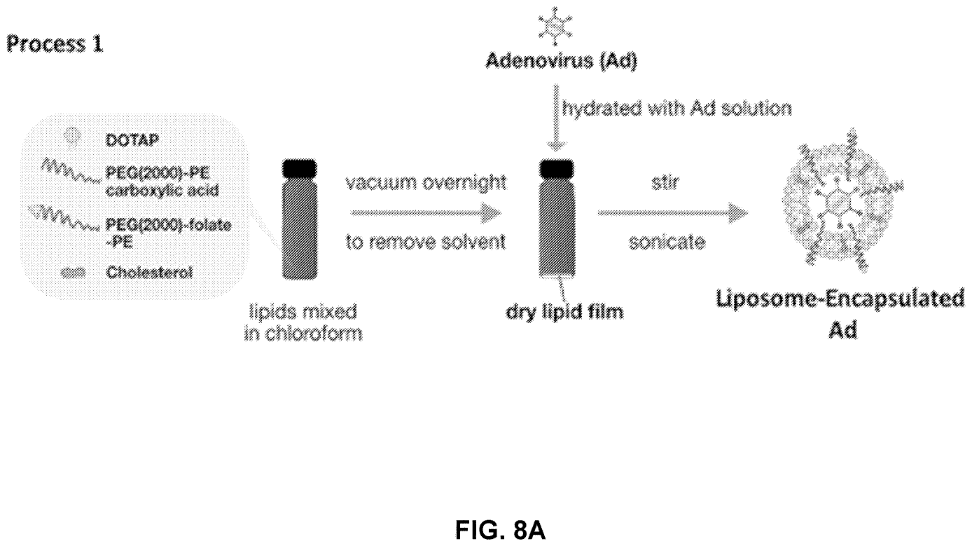

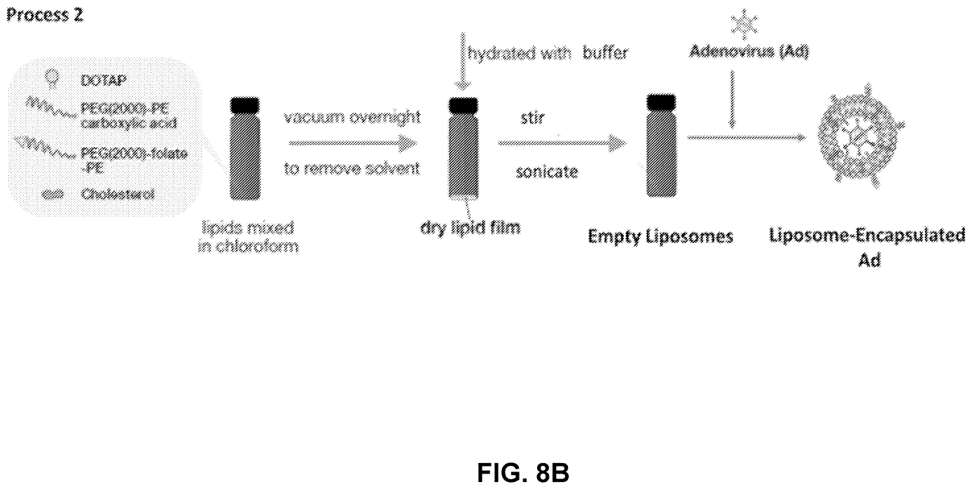

Encapsulated adenovirus transfects CAR⁺ and CAR⁻ cells. The synthesis of a lipid-coated Ad is illustrated in FIGS. 8A-8B, Processes 1 and 2. A lipid film composed of DOTAP, cholesterol, 1,2-distearoyl-sn-glycero-3-phosphoethanolamine-N-[carboxy (polyethylene glycol)-2000] (PEG (2000)-PE carboxylic acid), and 1,2-distearoyl-sn-glycero-3-phosphoethanolamine-N-[folate (polyethylene glycol)-2000] (PEG (2000)-folate-PE) was used to encapsulate adenovirus (Ad), producing Ad-DOTAP-folate liposomes (Ad-Df). 1/1×, ⅓×, ⅕×, 1/7×, and 1/10×Ad-Df represent the Ad viral particle to DOTAP lipid (nmol) ratios of 5.17×10⁶, 1.55×10⁷, 2.59×10⁷, 3.62×10⁷, and 5.17×10⁷, respectively. Production of adenovirus-encapsulated within liposomes was confirmed by cryogenic transmission electron microscopy. Characteristics of freshly prepared, empty liposomes and adenovirus-containing liposomes are provided in Table 1-1.

TABLE 1-1
Liposome Characteristics

| Liposomes | Average Diameter | Zeta Potential |
|---|---|---|
| (extruded 200 nm) | (nm) | (mV) |
| Df (empty) | 250.6 | −3.88 |
| | | (neutral) |
| Df-Ad (encapsulated) | 249.1 | −1.76 |
| | | (neutral) |

A recombinant human Ad expressing GFP (GFPAd) was used to determine the transfection efficiency by quantifying the percentage of GFP-positive cells after infection. HEK293 (human embryonic kidney cells), A549 (human lung cancer cells), MCF7 (human breast cancer cells), CT26 (mouse colon cancer cells), and THP-1 (human monocytes) cell lines with various expression levels of CAR were chosen to assess the transfection efficiency of naked GFPAd and encapsulated GFPAd (within DOTAP-folate liposome, GFPAd-Df). The levels of CAR expression were reported previously and were also examined by flow cytometry. Results are shown in FIGS. 1A-1E. HEK293 and A549 are CAR-positive cell lines and MCF7, CT26, and THP-1 are CAR-negative cell lines. From MOI=1.56 to 100, GFPAd and GFPAd-Df transfect CAR-positive cells, and there is no significant difference between naked GFPAd and GFPAd-Df, or among the different Ad-to-lipid ratio variants of GFPAd-Df (FIG. 1A-B). 1/1×GFPAd-Df on HEK293 cells demonstrated a reduced percentage of GFP-positive cells at MOI=100, which is likely because high concentrations of DOTAP are cytotoxic to cells. ⅓× to 1/10×GFPAd-Df showed a proportional increase in GFP-positive cells when increasing MOI.

In MCF7 and CT26 cell lines GFPAd infection of cells with low levels of CAR resulted in low levels of GFP expression, as expected (FIGS. 1C-1D). In contrast, GFPAd-Df-infected CAR-negative cells and synthesized high levels of GFP, which suggests that GFPAd encapsulated in liposomes can transfect cells by a CAR-independent manner. The percentage of GFP-positive cells was 4- to 32-fold higher in GFPAd-Df-transfected MCF7 than in GFPAd-transfected MCF7. Uncoated Ad only showed subtle GFP expression at high MOI (MOI>100) and was unable to detectably transfect CAR-negative cells at MOIs below MOI 25. On the other hand, GFPAd-Df was able to transfect CAR-negative cells at all MOIs examined. Folate in the liposome formulation is also attributed to some of the improvement of transfection efficiency in CAR-negative but FR-positive cells.

Additionally, inclusion of HSA at 20, 30 and 50 mg/mL was found to increase the CT26 cell transfection efficiency of GFP-Ad-Df about 2-fold at MOIs of 12.5, 25 and 50 as compared to formulations containing little to no HSA (e.g., 0-2 mg/mL). Therefore, high concentrations of HSA are thought to be advantageous for the intravenous administration of cationic liposome-encapsulated adenovirus.

Interestingly, inclusion of cholesterol did not appreciably alter the CT26 cell transfection efficiency of freshly prepared GFP-Ad-Df at a MOI of 100, but the CT26 cell transfection efficiency of stored (5 days at 4° C.) GFP-Ad-Df containing cholesterol was reduced. However, cholesterol is thought to be important for maintaining stability of liposomes in vivo and is therefore thought to be advantageous in the clinic. Inclusion of DSPE-PEG-2000 was found to be very important as the CT26 cell transfection efficiency of GFP-Ad-Df lacking DSPE-PEG-2000 was very poor under both freshly prepared and stored conditions.

Comparisons of the zeta-potential and transfection efficiency of different liposome formulations are provided in Table 1-2. Zeta-potential measurements were carried out in PBS (pH=7.4). Thus, formulations of liposome-encapsulated adenoviruses having a negative zeta potential or a less positive zeta potential are associated with higher transfection efficiencies.

TABLE 1-2
Properties of Empty Liposomes and Encapsulated Virus

| Formulation | | | | |
|---|---|---|---|---|
| Extruded 200 | Zeta-P (mv) | Zeta-P (mv) + | Zeta-P Delta | CT26 |
| nm | Empty | Ad | (mv) | Efficiency\hat{ } |
| Optimized | −3.88 | −1.76 | +2.12 | 1X |
| Cholesterol | | | | |
| No | −0.17 | +3.48 | +3.31 | ~1X |
| Cholesterol | | | | |
| 150% | +4.16 | +2.56 | +1.60 | ~1X |
| Cholesterol | | | | |
| No | +29.30 | +39.03 | −0.27 | ~0.3X |
| DSPE-PEG- | | | | |
| Acid | | | | |
| No | +6.58 | +8.74 | +2.16 | ~0.5X |
| DSPE-PEG- | | | | |
| Folate | | | | |
| Optimized | −5.62 | −4.40 | +1.22 | ~2X |
| Df + HSA | | | | |

\hat{ }CT26 transfection efficiency is shown in comparison to liposomes containing an optimal cholesterol concentration.

Since GFPAd-Df was shown to transfect CAR-deficient epithelial cancer cells, the transfection capability in other cell types was also investigated. In contrast to adherent cells, suspension cells and particularly hematopoietic cells, are notoriously difficult to transfect. THP-1, a human monocytic leukemia cancer and CAR-deficient cell line was used to estimate the transfection capability of GFPAd-Df in suspensions of hematopoietic cells. THP-1 cells were transfected with naked GFPAd and GFPAd-Df at MOI 25-200 (FIG. 1E). A negatively charged liposome coated GFPAd was used as a control to compare the transfection efficiency of encapsulated GFPAd. The transfection efficiency of negatively charged liposome encapsulated GFPAd was improved compared to naked GFPAd in THP-1 cells (CAR-negative). The transfection performed with cationic 1/10×GFPAd-Df showed the strongest GFP expression among all groups and raised the number of GFP-positive cells more than 10× compared to naked GFPAd. Encapsulated GFPAd strongly enhances GFPAd entry into CAR-deficient cancer cells and allows transfer of viral genes with MOIs below 25. The positive-charge of the liposomes most likely interacts with the negatively charged cell surface to increase the probability of cell internationalization. These results show that GFPAd poorly transfects cells without CAR expressed on the cell surface, and liposome-encapsulation can eliminate the strong dependency on CAR expression for cellular entry. Additionally, GFPAd-Df can effectively transfect various types of cells, which include epithelial cancer cells and suspended hematopoietic cells.

Encapsulated adenovirus efficiently transfects primary breast tumor-derived cells. To model the diverse landscape of human cancers, patient-derived tumor cells were employed as a preclinical model to study the transfection efficiency of the non-encapsulated and encapsulated Ad. Replication-defective GFPAd was used to determine the transfection efficiency by quantifying the percentage of GFP-positive cells after infection. Breast tumor tissues were surgically removed from four patients and dissociated at the laboratory to form single cell suspensions. Patient-derived single cells were plated and incubated at 37° C. in 5% CO₂ for 1-2 days. After cells adhered to the plate, they were transfected with GFPAd, ⅓×GFPAd-Df, or 1/10×GFPAd-Df at MOI=100 and the transfection efficiency in primary human tumor cells was quantified by counting the percentage of GFP-positive cells. A relatively high Ad to lipid ratio formula (⅓×GFPAd-Df) and low Ad to lipid ratio formula ( 1/10×GFPAd-Df) were used to confirm that the transfection performance in primary cells is similar to that in cell lines. 1/1×GFPAd-Df was not chosen due to potential cytotoxicity at high MOI. The ⅓×GFPAd-Df, 1/10×GFPAd-Df, and GFPAd showed 72%, 82% and 26% of GFP-positive breast tumor cells, respectively (FIG. 3A). Flow cytometry analysis indicated that most cells derived from the breast cancer patients are CAR deficient (FIG. 3B). These data show that GFPAd-Df is capable of transfecting primary human tumor cells, which do not express CAR on the cell surface. In summary, Ad encapsulated in DOTAP-folate liposome is suitable for clinical use because it promotes effective and broad transfection of cancer cells.

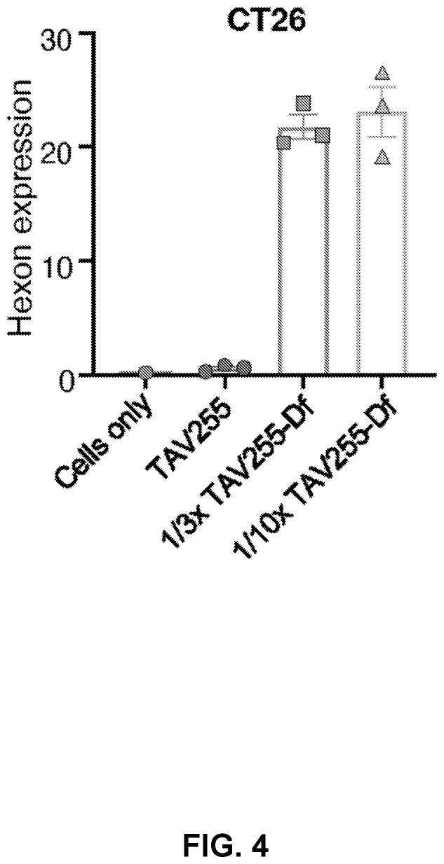

Replicating adenovirus encapsulated in liposomes transfects CAR-negative cells. After showing that encapsulated Ad can transfect CAR-deficient cells using replication-deficient GFPAd, a replicating Ad was used with the same liposome formulations to ensure the replicating virus is able to undergo liposomal delivery in a similar way. TAV255, a tumor-selective replicating oncolytic adenovirus, was encapsulated in DOTAP-folate liposomes to evaluate the transfection efficiency in CAR-deficient cells. TAV255 has a 50 nucleotide deletion in the promoter of E1A and it showed potent tumor-selective oncolytic activity in CAR-positive cells in vitro and in vivo. Hexon, the most abundant structural protein in Ad capsid, was used to quantify the transfection efficiency. CAR-negative CT26 cells were transfected with TAV255, ⅓×TAV255-Df and 1/10×TAV255-Df at MOI=400 and hexon was stained for visualization. ⅓×TAV255-Df and 1/10×TAV255-Df displayed an increased amount of hexon protein compared to TAV255. Hexon expression was analyzed and quantified using ImageJ Fiji software (FIG. 4). ⅓×TAV255-Df and 1/10×TAV255-Df had a 26- and 28-fold increase in hexon expression in CT26 cells compared to non-encapsulated TAV255. These results indicate the replicating virus encapsulated in liposomes showed improved adenoviral transfection efficiency. Since hexon is encoded by viral late genes, the hexon expression in TAV255-Df-infected cells suggests that TAV255-Df was able to reach the late-stage of viral gene transcription. The enhanced transfection efficiency of the replicating virus in liposomes indicates that such a coating formulation can be used for cancer treatments to deliver oncolytic viruses to tumor cells, resulting in expression of viral genes and viral replication within the tumor cells, which eventually leads to destruction of tumor cells.

Viability of CAR⁺ and CAR⁻ cells transfected with replicating adenovirus encapsulated in liposomes. To verify that the encapsulated replicating Ad (TAV255) can kill tumor cells after transfection, an Alamar Blue assay was used to determine cell viability after transfection. Both TAV255 and encapsulated TAV255 kill CAR-positive tumor cells, such as A549 (FIG. 5). The killing ability is correlated with incubation time and MOI. In CAR-negative tumor cells, such as CT26, TAV255 showed limited killing ability for tumor cells, while encapsulated TAV255 (⅓× and 1/10×TAV255-Df) demonstrated enhanced tumor cell killing ability (FIG. 5). On day 6 post-infection, 1/10×TAV255-Df had a 5-fold and 2-fold higher killing ability than naked TAV255 at MOI=400 and MOI=200, respectively. Encapsulating TAV255 in liposomes overcomes the limited clinical applicability of conventional oncolytic adenovirus. Taken together the results from FIG. 4 and FIG. 5, show that TAV255-Df can effectively transfect and kill CAR-negative tumor cells, providing a powerful alternative to naked TAV255.

Encapsulated adenovirus suppresses tumor growth. Four treatment groups were used to elucidate the effect of virus encapsulation: (1) vehicle, (2) empty liposome ( 1/10×Df), (3) naked adenovirus (TAV255), and (4) coated adenovirus ( 1/10×TAV255-Df). 1/10×TAV255 was intratumorally injected every other day for a total of six doses. Tumors were harvested on day 12 after the first treatment to investigate the tumor infiltrating lymphocytes (TIL) in the tumor environment (FIG. 6, top row ((a)-(d))). The 1/10×TAV255-Df treated group showed a higher number of infiltrated killer T cells (CD45⁺CD8⁺) in the tumors compared to the other treatments. About a 10-fold increase in in IFN-γ⁺-killer T cells (CD45⁺CD8⁺IFN-γ⁺) was observed in tumors following 1/10×TAV255-Df therapy, indicative of the activation of cytotoxic T cells to destroy tumor cells.

The increase in immune cells and IFN-γ⁺ T cells in tumors indicates an improved immune response brought about by 1/10×TAV255-Df therapy. In Study 1, the enhanced immune response and tumor cell killing ability was also reflected in the inhibition of tumor growth (FIG. 6, bottom row ((e)-(g)). The 1/10×TAV255-Df treated group demonstrated the best inhibition of tumor growth. Four out of six CT26 tumors were induced into full remission by 1/10×TAV255-Df treatment (~67% full remission rate), compared to two out of six tumors that were induced into full remission by TAV-255 (~33% full remission rate). In addition, the 1/10×TAV255-Df treatment significantly improved survival rates (FIG. 6, rightmost bottom row (h)). On day 38 post-treatment, 83% of mice were alive in 1/10×TAV255-Df treated group; while 33% and 0% of mice were alive in TAV255 and PBS treated groups, respectively.

Likewise in Study 2, 0 of the 8 mice treated with intratumoral injections of PBS were cured, while 2 of 6 mice treated with TAV and 4 of 6 mice treated with encapsulated TAV (DfTAV) had complete anti-tumor responses (Table 1-3 and FIGS. 10A-10B). Strikingly, the mice with complete responses showed no signs of recurrence of the original tumor throughout the remainder of the study.

Surviving mice from the TAV and DfTAV groups (n=6) were re-challenged with CT26 (colon cancer) cells on the opposite flank two weeks after termination of treatment, and as a control, naïve age-matched mice (n=5) were implanted with CT26 cells. Tumor growth was monitored, and the number of tumor free mice was determined. As shown in Table 1-3, tumors appeared in all 5 naïve mice as expected, while 4 of 4 mice treated with DfTAV and 2 of 2 mice treated with TAV were protected from CT26 re-challenge. Survival curves were analyzed using a long rank test. Due to the small number of subjects in these studies, p>0.05 is non-significant.

In further experiments, the CT26-experienced mice were injected 4 weeks with later with a 4T1 (breast cancer) cells, to investigate the potential of systemic immunity against a different tumor. Importantly, 1 of 2 mice treated with TAV and 4 of 4 mice treated with DfTAV were fully protected after challenge with 4T1 cells, while 0 of 4 mice treated with PBS were not protected. This was unexpected, given that the CT26-experienced mice had never been exposed to 4T1 tumor cells before and therefore were not contemplated to be resistant to 4T1 tumor cell growth. These results indicate that there is some cross-reactivity between the two tumor cell lines and/or, that tumor cell death from intratumoral injection with an adenovirus (e.g., TAV) releases common tumor antigens, such as tumor-associated antigens and/or cancer-germline antigens, which trigger cross-reactive T cell responses and the development of broad immunity against different tumors.

TABLE 1-3
Efficacy of Adenovirus-Treatment\hat{ }

| | | Tumor-Free | Tumor-Free |
|---|---|---|---|
| Treatment | Survivors | CT26 Re-Challenge | 4T1 Re-Challenge |
| Control (PBS) | 0/8 | 0/5 | 0/4 |
| TAV | 2/6 | 2/2 | 1/2 |
| DfTAV | 4/6 | 4/4 | 4/4 |

\hat{ }Survival was determined on day of CT26 re-challenge, tumor-free post CT26 re-challenge was determined on day of 4T1 re-challenge, and tumor-free post 4T1 re-challenge was determined at conclusion of the study (about day 180).

A comparison of unencapsulated vs. encapsulated TAV255 on CAR-deficient tumor growth and remission is shown in FIG. 11B. Treatment schedule: pbs, empty liposome (df), tav255, and tav255-df (n=10-24) were intratumorally injected every other day. Survivors with full remission were rechallenged by CT-26 tumors on day 64. (11b-1) survival curves (vehicle n=10, empty liposomes n=10, unencapsulated adenovirus tav255 n=24 and liposomes encapsulated tav255-df n=24) with initial challenge of CT-26 tumor on the right flank of the mice and rechallenge of the tumors on the left flank. (11b-2) 33% of the mice had complete remission from the treatment of TAV255, and 58% of the mice had complete remission by TAV255-df. (11b-3) 8/8 and 13/13 mice from tav255 and tav255-df treatments survived from the rechallenge. No mice from the control group survived rechallenge.

In summary, liposomal encapsulation of attenuated adenovirus improves survival compared to non-encapsulated (“naked”) attenuated adenovirus and the long-term memory immune response induced by the viral therapy provides protection against tumor cell re-challenge at least in a subset of mice.

### Example 2. Effects of Liposome Encapsulation of Adenovirus on Protection from Neutralizing Antibodies and Transfection Efficiency

This example describes comparisons of encapsulated adenovirus and naked adenovirus grown in HEK293 (human embryonic kidney) cells in the presence or absence of human serum containing adenovirus-neutralizing antibodies.

### Adenovirus Neutralization

Human plasma containing a high titer of neutralization antibodies was stored at −80° C. until use. HEK293 cells were plated at 10,000 cells/well in a 96-well plate overnight. Non-encapsulated TAV-adenovirus encoding green fluorescent protein (GFP) protein (Ad (GFP)), liposomal AdGFP+folate−PEG, liposomal AdGFP+PEG−folate and liposomal AdGFP+PEG+folate were incubated with anti-adenovirus whole antiserum for 1 hour at 37° C. diluted to 1:10 followed by 4-fold serial dilutions. Plasma was first decomplemented for 30 min at 56° C. 10 μl of plasma was added to cells at corresponding concentrations up to 1:10240 followed by addition of AdGFP, AdGFP+folate−PEG, AdGFP+PEG−folate and AdGFP+PEG+folate. Samples were incubated with cells for 24 hours at 37° C. and 5% CO₂. Cells were re-suspended in 100 μl of 1×PBS and fluorescence intensities were measured using a Tecan Infinite M200 microplate reader at an excitation 2 of 480 nm and an emission 2 of 520 nm.

As shown in FIG. 12A, naked AdGFP, liposomal AdGFP+folate-PEG, liposomal AdGFP+PEG-folate and liposomal AdGFP+PEG+folate in human plasma were assayed at six dilutions, 1:10, 1:40, 1:160, 1:640, 1:2560, 1:10240 and no antibody. The GFP gene, which presents in the viral DNA of all four different preparations, served as a reporter or marker.

The assay readout, fluorescence intensity, was measured by a fluorescent based plate reader. These data indicate that liposomal encapsulation markedly protects AdTAV-255 (but does not, however, provide 100% protection possibly indicative of incomplete encapsulation) from neutralizing antibodies, which may prolong the virus persistence in vivo. PEG-containing liposomes show the best protection from nAb neutralization especially at more dilute concentrations; however, the degree of protection is directly proportional to the concentration of neutralizing antibodies. As a large hydrophilic polymer, PEG likely forms a protective water shell that sterically shields the liposome and hinders binding of nAbs.

### Transfection Efficiency

HEK293 human embryonic kidney cells were plated overnight at 1×10⁴ cells/well in a 96-well plate; cultures were incubated at 37° C. and 5% CO₂ in DMEM media supplemented with 10% FBS, 1% PSG. AdGFP, AdGFP+folate−PEG, AdGFP+PEG−folate and AdGFP+PEG+folate were added to cells and incubated for 48 hours at 37° C. and 5% CO₂. The cells were resuspended in 100 μl of 1×PBS and fluorescence intensities were measured using a Tecan Infinite M200 microplate reader at an excitation λ of 480 nm and an emission λ of 520 nm.

FIG. 12B shows the transfection efficiencies of unmodified and liposomal-modified Ads (with and without PEG and with and without folate) in the absence or presence of neutralizing antibody against Ad. The differential transfection efficiency was quantified by fluorescence intensity, a measure of the intensity of GFP expression per cell that corresponds to the extent to which each cell had been transfected.

Neutralizing antibody in human serum at a serum dilution of 1:10 was sufficient to reduce the relative fluorescence intensities resulting from infection with unmodified AdGFP, liposomal AdGFP+folate−PEG, liposomal AdGFP+PEG−folate and liposomal AdGFP+PEG+folate (FIG. 12A). However, PEG-containing liposomes provided the best protection from nAb neutralization likely due to a steric block of nAb binding.

This experiment demonstrates that liposome encapsulation protects from antibody neutralization, and increases transfection efficiency (hence, by extension, modifying the biodistribution of the TAV virus and the target cell range since previous experiments have shown that liposomal encapsulation permits the infection of CAR negative cells) especially when the liposome contains PEG.

FIG. 13 shows the transfection efficiencies of unmodified and liposomal-modified Ads in the absence or presence of neutralizing antibody against Ad in vivo. Mice (6 per group) were administered saline or saline with adenovirus (1e8 v.p. subcutaneously to induce an immune response to adenovirus in the mice. Three weeks later, mice were administered unencapsulated or encapsulated AdGFP IV. Blood was collected 5 minutes later and serum isolated and viral titer was evaluated by infection of HEK293 cells. The differential transfection efficiency was quantified by fluorescence intensity, a measure of the intensity of GFP expression per cell that corresponds to the extent to which each cell had been transfected. The results, shown in FIG. 13, demonstrate equal fluorescence intensity following IV injection of AdGFP in unimmunized (PBS) control mice (lanes 1 and 2). In contrast, the fluorescence intensity in mice immunized with adenovirus (TAV-255) was significantly reduced in the mice with unencapsulated virus (lane 4) but remained at control levels of fluorescence intensity in the mice treated with encapsulated virus (lane 3).

FIG. 14 shows the expression of adenoviral E1a mRNA in tumor tissue following IV administration.

Although the foregoing disclosure has been described in some detail by way of illustration and example for purposes of clarity and understanding, it will be apparent to those skilled in the art that certain changes and modifications may be practiced. Therefore, the examples should not be construed as limiting the scope of the disclosure, which is delineated by the appended claims.

What is claimed is:

1. A method of preparing a cationic liposome encapsulating an adenovirus particle, the method comprising:
a) preparing a lipid mixture by dissolving 1,2-dioleoyl-3-trimethylammonium-propane (DOTAP), a pegylated carboxylic acid-terminated phospholipid, and a pegylated folate-containing phospholipid in an organic solvent;
b) drying the lipid mixture under vacuum to form a dry lipid film;
c) hydrating the dried lipid film by contacting the film with a pharmaceutically acceptable buffer to form a liquid suspension comprising a multilamellar vesicle;
d) physically disrupting the multilamellar vesicle to form an empty cationic liposome; and
e) adding an adenovirus particle to the empty cationic liposome to prepare a cationic liposome encapsulating the adenovirus particle.

2. A method of preparing a cationic liposome encapsulating an adenovirus particle, the method comprising:
a) preparing a lipid mixture by dissolving 1,2-dioleoyl-3-trimethylammonium-propane (DOTAP), a pegylated carboxylic acid-terminated phospholipid, and a pegylated folate-containing phospholipid in an organic solvent;
b) drying the lipid mixture under vacuum to form a dry lipid film;
c) hydrating the dried lipid film by contacting the film with a pharmaceutically acceptable buffer comprising an adenovirus particle to form a liquid suspension comprising a multilamellar vesicle encapsulating the adenovirus particle; and
d) physically-disrupting the multilamellar vesicle to prepare a cationic liposome encapsulating the adenovirus particle.

3. A method of preparing a cationic liposome encapsulating an adenovirus particle, the method comprising:
a) preparing a lipid mixture by dissolving 1,2-dioleoyl-3-trimethylammonium-propane (DOTAP), cholesterol, a pegylated carboxylic acid-terminated phospholipid, and a pegylated folate-containing phospholipid in an organic solvent;
b) drying the lipid mixture under vacuum to form a dry lipid film;
c) hydrating the dried lipid film by contacting the film with a pharmaceutically acceptable buffer to form a liquid suspension comprising a multilamellar vesicle;
d) physically disrupting the multilamellar vesicle to form an empty cationic liposome; and
e) adding an adenovirus particle to the empty cationic liposome to prepare a cationic liposome encapsulating the adenovirus particle.

4. A method of preparing a cationic liposome encapsulating an adenovirus particle, the method comprising:
a) preparing a lipid mixture by dissolving 1,2-dioleoyl-3-trimethylammonium-propane (DOTAP), cholesterol, a pegylated carboxylic acid-terminated phospholipid, and a pegylated folate-containing phospholipid in an organic solvent;
b) drying the lipid mixture under vacuum to form a dry lipid film;
c) hydrating the dried lipid film by contacting the film with a pharmaceutically acceptable buffer comprising an adenovirus particle to form a liquid suspension comprising a multilamellar vesicle encapsulating the adenovirus particle; and
d) physically-disrupting the multilamellar vesicle to prepare a cationic liposome encapsulating the adenovirus particle.

5. A method of preparing a cationic liposome encapsulating an adenovirus particle, the method comprising:
a) preparing a lipid mixture by dissolving 1,2-dioleoyl-3-trimethylammonium-propane (DOTAP), cholesterol, a pegylated carboxylic acid-terminated phospholipid, and a pegylated folate-containing phospholipid in an organic solvent;
b) microfluidizing the lipid mixture with a pharmaceutically acceptable buffer in a to form a composition comprising an empty cationic liposome in an organic solvent;
c) evaporating the organic solvent from the composition leaving the empty cationic liposome; and
d) adding an adenovirus particle to the empty cationic liposome to prepare a cationic liposome encapsulating the adenovirus particle.

6. A method of preparing a cationic liposome encapsulating an adenovirus particle, the method comprising:
a) preparing a lipid mixture by dissolving 1,2-dioleoyl-3-trimethylammonium-propane (DOTAP), cholesterol, a pegylated carboxylic acid-terminated phospholipid, and a pegylated folate-containing phospholipid in an organic solvent;
b) microfluidizing the lipid mixture with a pharmaceutically acceptable buffer comprising an adenovirus particle to form a composition comprising a cationic liposome encapsulating the adenovirus particle in an organic solvent;
c) evaporating the organic solvent from the composition leaving the cationic liposome encapsulating the adenovirus particle.

7. The method of any one of claims 1-6, wherein the cationic liposome encapsulating the adenovirus particle is a unilamellar vesicle of about 100 to about 900 nm in diameter, optionally about 100 to about 300 nm in diameter.

8. The method of any one of claims 1-7, further comprising lyophilizing the cationic liposome encapsulating the adenovirus particle.

9. The method of any one of claims 1-8, wherein
the pegylated carboxylic acid-terminated phospholipid is a 1,2-diacyl-sn-glycero-3-phosphoethanolamine-N-[carboxy (polyalkylene glycol)-(MW)], or a pharmaceutically acceptable salt thereof, and
the pegylated folate-containing phospholipid is a 1,2-diacyl-sn-glycero-3-phosphoethanolamine-N-[folate (polyalkylene glycol)-(MW)], and
wherein MW is a molecular weight in the range of about 1000-10000 Daltons, optionally in the range of about 1000-5000 Daltons, optionally about 2000 Daltons.

10. The method of claim 8, wherein
the pegylated carboxylic acid-terminated phospholipid is a pharmaceutically acceptable salt of 1,2-distearoyl-sn-glycero-3-phosphoethanolamine-N-[carboxy (polyethylene glycol)-2000] (DSPE-PEG (2000) carboxylic acid), and
the pegylated folate-containing phospholipid is a pharmaceutically acceptable salt of 1,2-distearoyl-sn-glycero-3-phosphoethanolamine-N-[folate (polyethylene glycol)-2000] (DSPE-PEG (2000) folate).

11. The method of any one of claims 1-10, wherein the organic solvent comprises chloroform.

12. The method of any one of claims 1-10, wherein the organic solvent comprises methanol, ethanol, isopropanol, or a combination thereof.

13. The method of any one of claims 1-12, wherein the pharmaceutically acceptable buffer is a sterile solution with a pH in the range of about 7.0 to 8.0, optionally wherein the pH is from 7.3 to 7.5, optionally wherein the pH is about 7.4.

14. The method of claim 13, wherein the pharmaceutically acceptable buffer is phosphate buffered saline (PBS), tris(hydroxymethyl)aminomethane buffer (Tris), or (4-(2-hydroxyethyl)-1-piperazineethanesulfonic acid buffer (HEPES), optionally wherein the pharmaceutically acceptable buffer is PBS.

15. The method of any one of claims 3-14, wherein the molar ratio of the cholesterol to the DOTAP in step a) is 0.02-0.40 to 1.0 (0.02-0.40:1.0), optionally wherein the molar ratio is 0.26 to 1.0.

16. The method of any one of claims 1-15, wherein the molar ratio of the pegylated folate-containing phospholipid to the DOTAP in step a) is 0.01-0.02 to 1.0 (001-0.02:1.0), optionally wherein the molar ratio is 0.01 to 1.0.

17. The method of any one of claims 1-16, wherein the molar ratio of the pegylated carboxylic acid-terminated phospholipid to the DOTAP in step a) is 1.0 to 0.02-0.04 (1.0:02-0.04), optionally wherein the molar ratio is 1.0 to 0.02.

18. The method of any one of claims 1-17, wherein the molar ratio of the DOTAP, the cholesterol, the pegylated carboxylic acid-terminated phospholipid, and the pegylated folate-containing phospholipid in step a) is about 1.0:0.26:0.02:0.01.

19. The method of any one of claims 1-18, wherein the adenovirus particle to DOTAP lipid ratio is about 5×10⁶ to about 5×10⁸ viral particles: nmol (vp/nM).

20. The method of claim 19, wherein the adenovirus particle to DOTAP lipid ratio is about 1×10⁷ to about 1×10⁸ vp/nM, optionally wherein the ratio is about 5×10⁷ vp/nM, or wherein the ratio is 5.17×10⁷ vp/nM.

21. The method of any one of claims 1-20, wherein the adenovirus is a recombinant human adenovirus of serotype 5 (Ad5).

22. The method of any one of claims 1-21, wherein the adenovirus is an oncolytic adenovirus.

23. A pharmaceutical formulation comprising an excipient, and a plurality of the cationic liposome-encapsulated adenovirus particles produced by the method of any one of claims 1-22.

24. A method of treating a cancer in a patient, the method comprising administering an effective mount of the pharmaceutical formulation of claim 23 to the patient to treat the cancer.

25. The method of claim 24, wherein cells of the cancer are coxsackievirus-adenovirus receptor (CAR) negative.

26. A method of determining margins of a tumor in a surgical patient, the method comprising:
a) administering an effective mount of the pharmaceutical formulation of claim 23 to the surgical patient;
b) allowing time for the adenovirus particles to enter into and replicate within cells of the tumor thereby producing a plurality of adenovirus progeny; and
c) detecting the adenovirus progeny thereby determining margins of the tumor, wherein the adenovirus particles express a detectable protein when replicating within cells of the tumor.

27. A pharmaceutical formulation comprising an excipient, and a plurality of cationic liposome-encapsulated adenovirus particles, wherein the cationic liposome is a large unilamellar vesicle comprising 1,2-dioleoyl-3-trimethylammonium-propane (DOTAP), a pegylated carboxylic acid-terminated phospholipid, and a pegylated folate-containing phospholipid, and wherein each of the cationic liposome-encapsulated adenovirus particles is about 100 to about 900 nm in diameter, optionally about 100 to about 300 nm in diameter.

28. A method of transfecting mammalian cells, the method comprising:
a) contacting a mammalian cell with an effective amount of the pharmaceutical formulation of claim 23 or claim 27; and
b) culturing the mammalian cell in culture media under conditions suitable for the adenovirus particle to enter into and replicate within cells of the tumor thereby producing a plurality of adenovirus progeny.


SEQUENCE LISTING (3 sequences)

SEQ ID NO: 1 - DNA, 35938 bp, Human adenovirus 5
  source 1..35938: mol_type = unassigned DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 2 - DNA, 10 bp, synthetic construct
  misc_feature 1..10: note = Modified E1a promoter
  source 1..10: mol_type = other DNA
ggtgttttgg

SEQ ID NO: 3 - DNA, 34794 bp, Human adenovirus 35
  source 1..34794: mol_type = unassigned DNA
catcatcaataatataccttatagatggaatggtgccaatatgtaaatgaggtgattttaaaaagtgtgggccgtgtggtgattggctgtggggttaacggttaaaaggggcggcgcggccgtgggaaaatgacgttttatgggggtggagtttttttgcaagttgtcgcgggaaatgttacgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatttatacctcagggtttgtgtcaagaggccactcttgagtgccagcgagaagagttttctcctctgcgccggcagtttaataataaaaaaatgagagatttgcgatttctgcctcaggaaataatctctgctgagactggaaatgaaatattggagcttgtggtgcacgccctgatgggagacgatccggagccacctgtgcagctttttgagcctcctacgcttcaggaactgtatgatttagaggtagagggatcggaggattctaatgaggaagctgtgaatggcttttttaccgattctatgcttttagctgctaatgaaggattagaattagatccgcctttggacactttcaatactccaggggtgattgtggaaagcggtacaggtgtaagaaaattacctgatttgagttccgtggactgtgatttgcactgctatgaagacgggtttcctccgagtgatgaggaggaccatgaaaaggagcagtccatgcagactgcagcgggtgagggagtgaaggctgccaatgttggttttcagttggattgcccggagcttcctggacatggctgtaagtcttgtgaatttcacaggaaaaatactggagtaaaggaactgttatgttcgctttgttatatgagaacgcactgccactttatttacagtaagtgtgtttaagttaaaatttaaaggaatatgctgtttttcacatgtatattgagtgtgagttttgtgcttcttattataggtcctgtgtctgatgctgatgaatcaccatctcctgattctactacctcacctcctgatattcaagcacctgttcctgtggacgtgcgcaagcccattcctgtgaagcttaagcctgggaaacgtccagcagtggagaaacttgaggacttgttacagggtggggacggacctttggacttgagtacacggaaacgtccaagacaataagtgttccatatccgtgtttacttaaggtgacgtcaatatttgtgtgagagtgcaatgtaataaaaatatgttaactgttcactggtttttattgctttttgggcggggactcaggtatataagtagaagcagacctgtgtggttagctcataggagctggctttcatccatggaggtttgggccattttggaagaccttaggaagactaggcaactgttagagagcgcttcggacggagtctccggtttttggagattctggttcgctagtgaattagctagggtagtttttaggataaaacaggactataaacaagaatttgaaaagttgttggtagattgcccaggactttttgaagctcttaatttgggccatcaggttcactttaaagaaaaagttttatcagttttagacttttcaaccccaggtagaactgctgctgctgtggcttttcttacttttatattagataaatggatcccgcagactcatttcagcaggggatacgttttggatttcatagccacagcattgtggagaacatggaaggttcgcaagatgaggacaatcttaggttactggccagtgcagcctttgggtgtagcgggaatcctgaggcatccaccggtcatgccagcggttctggaggaggaacagcaagaggacaacccgagagccggcctggaccctccagtggaggaggcggagtagctgacttgtctcctgaactgcaacgggtgcttactggatctacgtccactggacgggataggggcgttaagagggagagggcatccagtggtactgatgctagatctgagttggctttaagtttaatgagtcgcagacgtcctgaaaccatttggtggcatgaggttcagaaagagggaagggatgaagtttctgtattgcaggagaaatattcactggaacaggtgaaaacatgttggttggagccagaggatgattgggcggtggccattaaaaattatgccaagatagctttgaggcctgataaacagtataagatcagtagacggattaatatccggaatgcttgttacatatctggaaatggggctgaggtggtaatagatactcaagacaagacagttattagatgctgcatgatggatatgtggcctggagtagtcggtatggaagcagtcacttttgtaaatgttaagtttaggggagatggttataatggaatagtgtttatggccaataccaaacttatattgcatggttgtagcttttttggtttcaacaatacctgtgtagatgcctggggacaggttagtgtacgggggtgtagtttctatgcgtgttggattgccacagctggcagaaccaagagtcaattgtctctgaagaaatgcatattccaaagatgtaacctgggcattctgaatgaaggcgaagcaagggtccgtcactgcgcttctacagatactggatgttttattttaattaagggaaatgccagcgtaaagcataacatgatttgtggtgcttccgatgagaggccttatcaaatgctcacttgtgctggtgggcattgtaatatgctggctactgtgcatattgtttcccatcaacgcaaaaaatggcctgtttttgatcacaatgtgttgaccaagtgcaccatgcatgcaggtgggcgtagaggaatgtttatgccttaccagtgtaacatgaatcatgtgaaagtgttgttggaaccagatgccttttccagaatgagcctaacaggaatctttgacatgaacacgcaaatctggaagatcctgaggtatgatgatacgagatcgagggtgcgcgcatgcgaatgcggaggcaagcatgccaggttccagccggtgtgtgtagatgtgaccgaagatctcagaccggatcatttggttattgcccgcactggagcagagttcggatccagtggagaagaaactgactaaggtgagtattgggaaaactttggggtgggattttcagatggacagattgagtaaaaatttgttttttctgtcttgcagctgacatgagtggaaatgcttcttttaaggggggagtcttcagcccttatctgacagggcgtctcccatcctgggcaggagttcgtcagaatgttatgggatctactgtggatggaagacccgttcaacccgccaattcttcaacgctgacctatgctactttaagttcttcacctttggacgcagctgcagccgctgccgccgcctctgtcgccgctaacactgtgcttggaatgggttactatggaagcatcgtggctaattccacttcctctaataacccttctacactgactcaggacaagttacttgtccttttggcccagctggaggctttgacccaacgtctgggtgaactttctcagcaggtggccgagttgcgagtacaaactgagtctgctgtcggcacggcaaagtctaaataaaaaaaattccagaatcaatgaataaataaacgagcttgttgttgatttaaaatcaagtgtttttatttcatttttcgcgcacggtatgccctggaccaccgatctcgatcattgagaactcggtggattttttccagaatcctatagaggtgggattgaatgtttagatacatgggcattaggccgtctttggggtggagatagctccattgaagggattcatgctccggggtagtgttgtaaatcacccagtcataacaaggtcgcagtgcatggtgttgcacaatatcttttagaagtaggctgattgccacagataagcccttggtgtaggtgtttacaaaccggttgagctgggaggggtgcattcgaggtgaaattatgtgcattttggattggatttttaagttggcaatattgccgccaagatcccgtcttgggttcatgttatgaaggactaccaagacggtgtatccggtacatttaggaaatttatcgtgcagcttggatggaaaagcgtggaaaaatttggagacacccttgtgtcctccgagattttccatgcactcatccatgataatagcaatggggccgtgggcagcggcgcgggcaaacacgttccgtgggtctgacacatcatagttatgttcctgagttaaatcatcataagccattttaatgaatttggggcggagcgtaccagattggggtatgaatgttccttcgggccccggagcatagttcccctcacagatttgcatttcccaagctttcagttctgagggtggaatcatgtccacctggggggctatgaagaacaccgtttcgggggcgggggtgattagttgggatgatagcaagtttctgagcaattgagatttgccacatccggtggggccataaataattccgattacaggttgcaggtggtagtttagggaacggcaactgccgtcttctcgaagcaagggggccacctcgttcatcatttcccttacatgcatattttcccgcaccaaatccattaggaggcgctctcctcctagtgatagaagttcttgtagtgaggaaaagtttttcagcggttttagaccgtcagccatgggcattttggaaagagtttgctgcaaaagttctagtctgttccacagttcagtgatgtgttctatggcatctcgatccagcagacctcctcgtttcgcgggtttggacggctcctggagtagggtatgagacgatgggcgtccagcgctgccagggttcggtccttccagggtctcagtgttcgagtcagggttgtttccgtcacagtgaaggggtgtgcgcctgcttgggcgcttgccagggtgcgcttcagactcattctgctggtggagaacttctgtcgcttggcgccctgtatgtcggccaagtagcagtttaccatgagttcgtagttgagcgcctcggctgcgtggcctttggcgcggagcttacctttggaagttttcttgcataccgggcagtataggcatttcagcgcatacagcttgggcgcaaggaaaatggattctggggagtatgcatccgcgccgcaggaggcgcaaacagtttcacattccaccagccaggttaaatccggttcattggggtcaaaaacaagttttccgccatattttttgatgcgtttcttacctttggtctccataagttcgtgtcctcgttgagtgacaaacaggctgtccgtatctccgtagactgattttacaggcctcttctccagtggagtgcctcggtcttcttcgtacaggaactctgaccactctgatacaaaggcgcgcgtccaggccagcacaaaggaggctatgtgggaggggtagcgatcgttgtcaaccagggggtccaccttttccaaagtatgcaaacacatgtcaccctcttcaacatccaggaatgtgattggcttgtaggtgtatttcacgtgacctggggtccccgctgggggggtataaaagggggcggttctttgctcttcctcactgtcttccggatcgctgtccaggaacgtcagctgttggggtaggtattccctctcgaaggcgggcatgacctctgcactcaggttgtcagtttctaagaacgaggaggatttgatattgacagtgccggttgagatgcctttcatgaggttttcgtccatttggtcagaaaacacaatttttttattgtcaagtttggtggcaaatgatccatacagggcgttggataaaagtttggcaatggatcgcatggtttggttcttttccttgtccgcgcgctctttggcggcgatgttgagttggacatactcgcgtgccaggcacttccattcggggaagatagttgttaattcatctggcacgattctcacttgccaccctcgattatgcaaggtaattaaatccacactggtggccacctcgcctcgaaggggttcattggtccaacagagcctacctcctttcctagaacagaaagggggaagtgggtctagcataagttcatcgggagggtctgcatccatggtaaagattcccggaagtaaatccttatcaaaatagctgatgggagtggggtcatctaaggccatttgccattctcgagctgccagtgcgcgctcatatgggttaaggggactgccccagggcatgggatgggtgagagcagaggcatacatgccacagatgtcatagacgtagatgggatcctcaaagatgcctatgtaggttggatagcatcgcccccctctgatacttgctcgcacatagtcatatagttcatgtgatggcgctagcagccccggacccaagttggtgcgattgggtttttctgttctgtagacgatctggcgaaagatggcgtgagaattggaagagatggtgggtctttgaaaaatgttgaaatgggcatgaggtagacctacagagtctctgacaaagtgggcataagattcttgaagcttggttaccagttcggcggtgacaagtacgtctagggcgcagtagtcaagtgtttcttgaatgatgtcataacctggttggtttttcttttcccacagttcgcggttgagaaggtattcttcgcgatccttccagtactcttctagcggaaacccgtctttgtctgcacggtaagatcctagcatgtagaactgattaactgccttgtaagggcagcagcccttctctacgggtagagagtatgcttgagcagcttttcgtagcgaagcgtgagtaagggcaaaggtgtctctgaccatgactttgagaaattggtatttgaagtccatgtcgtcacaggctccctgttcccagagttggaagtctacccgtttcttgtaggcggggttgggcaaagcgaaagtaacatcattgaagagaatcttaccggctctgggcataaaattgcgagtgatgcggaaaggctgtggtacttccgctcgattgttgatcacctgggcagctaggacgatttcgtcgaaaccgttgatgttgtgtcctacgatgtataattctatgaaacgcggcgtgcctctgacgtgaggtagcttactgagctcatcaaaggttaggtctgtggggtcagataaggcgtagtgttcgagagcccattcgtgcaggtgaggatttgcatgtaggaatgatgaccaaagatctaccgccagtgctgtttgtaactggtcccgatactgacgaaaatgccggccaattgccattttttctggagtgacacagtagaaggttctggggtcttgttgccatcgatcccacttgagtttaatggctagatcgtgggccatgttgacgagacgctcttctcctgagagtttcatgaccagcatgaaaggaactagttgtttgccaaaggatcccatccaggtgtaagtttccacatcgtaggtcaggaagagtctttctgtgcgaggatgagagccgatcgggaagaactggatttcctgccaccagttggaggattggctgttgatgtgatggaagtagaagtttctgcggcgcgccgagcattcgtgtttgtgcttgtacagacggccgcagtagtcgcagcgttgcacgggttgtatctcgtgaatgagctgtacctggcttcccttgacgagaaatttcagtgggaagccgaggcctggcgattgtatctcgtgctcttctatattcgctgtatcggcctgttcatcttctgtttcgatggtggtcatgctgacgagcccccgcgggaggcaagtccagacctcggcgcgggaggggcggagctgaaggacgagagcgcgcaggctggagctgtccagagtcctgagacgctgcggactcaggttagtaggtagggacagaagattaacttgcatgatcttttccagggcgtgcgggaggttcagatggtacttgatttccacaggttcgtttgtagagacgtcaatggcttgcagggttccgtgtcctttgggcgccactaccgtacctttgttttttcttttgatcggtggtggctctcttgcttcttgcatgctcagaagcggtgacggggacgcgcgccgggcggcagcggttgttccggacccgggggcatggctggtagtggcacgtcggcgccgcgcacgggcaggttctggtattgcgctctgagaagacttgcgtgcgccaccacgcgtcgattgacgtcttgtatctgacgtctctgggtgaaagctaccggccccgtgagcttgaacctgaaagagagttcaacagaatcaatttcggtatcgttaacggcagcttgtctcagtatttcttgtacgtcaccagagttgtcctggtaggcgatctccgccatgaactgctcgatttcttcctcctgaagatctccgcgacccgctctttcgacggtggccgcgaggtcattggagatacggcccatgagttgggagaatgcattcatgcccgcctcgttccagacgcggctgtaaaccacggccccctcggagtctcttgcgcgcatcaccacctgagcgaggttaagctccacgtgtctggtgaagaccgcatagttgcataggcgctgaaaaaggtagttgagtgtggtggcaatgtgttcggcgacgaagaaatacatgatccatcgtctcagcggcatttcgctaacatcgcccagagcttccaagcgctccatggcctcgtagaagtccacggcaaaattaaaaaactgggagtttcgcgcggacacggtcaattcctcctcgagaagacggatgagttcggctatggtggcccgtacttcgcgttcgaaggctcccgggatctcttcttcctcttctatctcttcttccactaacatctcttcttcgtcttcaggcgggggcggagggggcacgcggcgacgtcgacggcgcacgggcaaacggtcgatgaatcgttcaatgacctctccgcggcggcggcgcatggtttcagtgacggcgcggccgttctcgcgcggtcgcagagtaaaaacaccgccgcgcatctccttaaagtggtgactgggaggttctccgtttgggagggagagggcgctgattatacattttattaattggcccgtagggactgcgcgcagagatctgatcgtgtcaagatccacgggatctgaaaacctttcgacgaaagcgtctaaccagtcacagtcacaaggtaggctgagtacggcttcttgtgggcgggggtggttatgtgttcggtctgggtcttctgtttcttcttcatctcgggaaggtgagacgatgctgctggtgatgaaattaaagtaggcagttctaagacggcggatggtggcgaggagcaccaggtctttgggtccggcttgctggatacgcaggcgattggccattccccaagcattatcctgacatctagcaagatctttgtagtagtcttgcatgagccgttctacgggcacttcttcctcacccgttctgccatgcatacgtgtgagtccaaatccgcgcattggttgtaccagtgccaagtcagctacgactctttcggcgaggatggcttgctgtacttgggtaagggtggcttgaaagtcatcaaaatccacaaagcggtggtaagcccctgtattaatggtgtaagcacagttggccatgactgaccagttaactgtctggtgaccagggcgcacgagctcggtgtatttaaggcgcgaataggcgcgggtgtcaaagatgtaatcgttgcaggtgcgcaccagatactggtaccctataagaaaatgcggcggtggttggcggtagagaggccatcgttctgtagctggagcgccaggggcgaggtcttccaacataaggcggtgatagccgtagatgtacctggacatccaggtgattcctgcggcggtagtagaagcccgaggaaactcgcgtacgcggttccaaatgttgcgtagcggcatgaagtagttcattgtaggcacggtttgaccagtgaggcgcgcgcagtcattgatgctctatagacacggagaaaatgaaagcgttcagcgactcgactccgtagcctggaggaacgtgaacgggttgggtcgcggtgtaccccggttcgagacttgtactcgagccggccggagccgcggctaacgtggtattggcactcccgtctcgacccagcctacaaaaatccaggatacggaatcgagtcgttttgctggtttccgaatggcagggaagtgagtcctattttttttttttttttgccgctcagatgcatcccgtgctgcgacagatgcgcccccaacaacagcccccctcgcagcagcagcagcagcaaccacaaaaggctgtccctgcaactactgcaactgccgccgtgagcggtgcgggacagcccgcctatgatctggacttggaagagggcgaaggactggcacgtctaggtgcgccttcgcccgagcggcatccgcgagttcaactgaaaaaagattctcgcgaggcgtatgtgccccaacagaacctatttagagacagaagcggcgaggagccggaggagatgcgagcttcccgctttaacgcgggtcgtgagctgcgtcacggtttggaccgaagacgagtgttgcgagacgaggatttcgaagttgatgaagtgacagggatcagtcctgccagggcacacgtggctgcagccaaccttgtatcggcttacgagcagacagtaaaggaagagcgtaacttccaaaagtcttttaataatcatgtgcgaaccctgattgcccgcgaagaagttacccttggtttgatgcatttgtgggatttgatggaagctatcattcagaaccctactagcaaacctctgaccgcccagctgtttctggtggtgcaacacagcagagacaatgaggctttcagagaggcgctgctgaacatcaccgaacccgaggggagatggttgtatgatcttatcaacattctacagagtatcatagtgcaggagcggagcctgggcctggccgagaaggtagctgccatcaattactcggttttgagcttgggaaaatattacgctcgcaaaatctacaagactccatacgttcccatagacaaggaggtgaagatagatgggttctacatgcgcatgacgctcaaggtcttgaccctgagcgatgatcttggggtgtatcgcaatgacagaatgcatcgcgcggttagcgccagcaggaggcgcgagttaagcgacagggaactgatgcacagtttgcaaagagctctgactggagctggaaccgagggtgagaattacttcgacatgggagctgacttgcagtggcagcctagtcgcagggctctgagcgccgcgacggcaggatgtgagcttccttacatagaagaggcggatgaaggcgaggaggaagagggcgagtacttggaagactgatggcacaacccgtgttttttgctagatggaacagcaagcaccggatcccgcaatgcgggcggcgctgcagagccagccgtccggcattaactcctcggacgattggacccaggccatgcaacgtatcatggcgttgacgactcgcaaccccgaagcctttagacagcaaccccaggccaaccgtctatcggccatcatggaagctgtagtgccttcccgatctaatcccactcatgagaaggtcctggccatcgtgaacgcgttggtggagaacaaagctattcgtccagatgaggccggactggtatacaacgctctcttagaacgcgtggctcgctacaacagtagcaatgtgcaaaccaatttggaccgtatgataacagatgtacgcgaagccgtgtctcagcgcgaaaggttccagcgtgatgccaacctgggttcgctggtggcgttaaatgctttcttgagtactcagcctgctaatgtgccgcgtggtcaacaggattatactaactttttaagtgctttgagactgatggtatcagaagtacctcagagcgaagtgtatcagtccggtcctgattacttctttcagactagcagacagggcttgcagacggtaaatctgagccaagcttttaaaaaccttaaaggtttgtggggagtgcatgccccggtaggagaaagagcaaccgtgtctagcttgttaactccgaactcccgcctgttattactgttggtagctcctttcaccgacagcggtagcatcgaccgtaattcctatttgggttacctactaaacctgtatcgcgaagccatagggcaaagtcaggtggacgagcagacctatcaagaaattacccaagtcagtcgcgctttgggacaggaagacactggcagtttggaagccactctgaacttcttgcttaccaatcggtctcaaaagatccctcctcaatatgctcttactgcggaggaggagaggatccttagatatgtgcagcagagcgtgggattgtttctgatgcaagagggggcaactccgactgcagcactggacatgacagcgcgaaatatggagcccagcatgtatgccagtaaccgacctttcattaacaaactgctggactacttgcacagagctgccgctatgaactctgattatttcaccaatgccatcttaaacccgcactggctgcccccacctggtttctacacgggcgaatatgacatgcccgaccctaatgacggatttctgtgggacgacgtggacagcgatgttttttcacctctttctgatcatcgcacgtggaaaaaggaaggcggtgatagaatgcattcttctgcatcgctgtccggggtcatgggtgctaccgcggctgagcccgagtctgcaagtccttttcctagtctacccttttctctacacagtgtacgtagcagcgaagtgggtagaataagtcgcccgagtttaatgggcgaagaggagtacctaaacgattccttgctcagaccggcaagagaaaaaaatttcccaaacaatggaatagaaagtttggtggataaaatgagtagatggaagacttatgctcaggatcacagagacgagcctgggatcatggggactacaagtagagcgagccgtagacgccagcgccatgacagacagaggggtcttgtgtgggacgatgaggattcggccgatgatagcagcgtgttggacttgggtgggagaggaaggggcaacccgtttgctcatttgcgccctcgcttgggtggtatgttgtgaaaaaaaataaaaaagaaaaactcaccaaggccatggcgacgagcgtacgttcgttcttctttattatctgtgtctagtataatgaggcgagtcgtgctaggcggagcggtggtgtatccggagggtcctcctccttcgtacgagagcgtgatgcagcagcagcaggcgacggcggtgatgcaatccccactggaggctccctttgtgcctccgcgatacctggcacctacggagggcagaaacagcattcgttactcggaactggcacctcagtacgataccaccaggttgtatctggtggacaacaagtcggcggacattgcttctctgaactatcagaatgaccacagcaacttcttgaccacggtggtgcagaacaatgactttacccctacggaagccagcacccagaccattaactttgatgaacgatcgcggtggggcggtcagctaaagaccatcatgcatactaacatgccaaacgtgaacgagtatatgtttagtaacaagttcaaagcgcgtgtgatggtgtccagaaaacctcccgacggtgctgcagttggggatacttatgatcacaagcaggatattttggaatatgagtggttcgagtttactttgccagaaggcaacttttcagttactatgactattgatttgatgaacaatgccatcatagataattacttgaaagtgggtagacagaatggagtgcttgaaagtgacattggtgttaagttcgacaccaggaacttcaagctgggatgggatcccgaaaccaagttgatcatgcctggagtgtatacgtatgaagccttccatcctgacattgtcttactgcctggctgcggagtggattttaccgagagtcgtttgagcaaccttcttggtatcagaaaaaaacagccatttcaagagggttttaagattttgtatgaagatttagaaggtggtaatattccggccctcttggatgtagatgcctatgagaacagtaagaaagaacaaaaagccaaaatagaagctgctacagctgctgcagaagctaaggcaaacatagttgccagcgactctacaagggttgctaacgctggagaggtcagaggagacaattttgcgccaacacctgttccgactgcagaatcattattggccgatgtgtctgaaggaacggacgtgaaactcactattcaacctgtagaaaaagatagtaagaatagaagctataatgtgttggaagacaaaatcaacacagcctatcgcagttggtatctttcgtacaattatggcgatcccgaaaaaggagtgcgttcctggacattgctcaccacctcagatgtcacctgcggagcagagcaggtttactggtcgcttccagacatgatgaaggatcctgtcactttccgctccactagacaagtcagtaactaccctgtggtgggtgcagagcttatgcccgtcttctcaaagagcttctacaacgaacaagctgtgtactcccagcagctccgccagtccacctcgcttacgcacgtcttcaaccgctttcctgagaaccagattttaatccgtccgccggcgcccaccattaccaccgtcagtgaaaacgttcctgctctcacagatcacgggaccctgccgttgcgcagcagtatccggggagtccaacgtgtgaccgttactgacgccagacgccgcacctgtccctacgtgtacaaggcactgggcatagtcgcaccgcgcgtcctttcaagccgcactttctaaaaaaaaaaaatgtccattcttatctcgcccagtaataacaccggttggggtctgcgcgctccaagcaagatgtacggaggcgcacgcaaacgttctacccaacatcccgtgcgtgttcgcggacattttcgcgctccatggggtgccctcaagggccgcactcgcgttcgaaccaccgtcgatgatgtaatcgatcaggtggttgccgacgcccgtaattatactcctactgcgcctacatctactgtggatgcagttattgacagtgtagtggctgacgctcgcaactatgctcgacgtaagagccggcgaaggcgcattgccagacgccaccgagctaccactgccatgcgagccgcaagagctctgctacgaagagctagacgcgtggggcgaagagccatgcttagggcggccagacgtgcagcttcgggcgccagcgccggcaggtcccgcaggcaagcagccgctgtcgcagcggcgactattgccgacatggcccaatcgcgaagaggcaatgtatactgggtgcgtgacgctgccaccggtcaacgtgtacccgtgcgcacccgtccccctcgcacttagaagatactgagcagtctccgatgttgtgtcccagcggcgaggatgtccaagcgcaaatacaaggaagaaatgctgcaggttatcgcacctgaagtctacggccaaccgttgaaggatgaaaaaaaaccccgcaaaatcaagcgggttaaaaaggacaaaaaagaagaggaagatggcgatgatgggctggcggagtttgtgcgcgagtttgccccacggcgacgcgtgcaatggcgtgggcgcaaagttcgacatgtgttgagacctggaacttcggtggtctttacacccggcgagcgttcaagcgctacttttaagcgttcctatgatgaggtgtacggggatgatgatattcttgagcaggcggctgaccgattaggcgagtttgcttatggcaagcgtagtagaataacttccaaggatgagacagtgtcaatacccttggatcatggaaatcccacccctagtcttaaaccggtcactttgcagcaagtgttacccgtaactccgcgaacaggtgttaaacgcgaaggtgaagatttgtatcccactatgcaactgatggtacccaaacgccagaagttggaggacgttttggagaaagtaaaagtggatccagatattcaacctgaggttaaagtgagacccattaagcaggtagcgcctggtctgggggtacaaactgtagacattaagattcccactgaaagtatggaagtgcaaactgaacccgcaaagcctactgccacctccactgaagtgcaaacggatccatggatgcccatgcctattacaactgacgccgccggtcccactcgaagatcccgacgaaagtacggtccagcaagtctgttgatgcccaattatgttgtacacccatctattattcctactcctggttaccgaggcactcgctactatcgcagccgaaacagtacctcccgccgtcgccgcaagacacctgcaaatcgcagtcgtcgccgtagacgcacaagcaaaccgactcccggcgccctggtgcggcaagtgtaccgcaatggtagtgcggaacctttgacactgccgcgtgcgcgttaccatccgagtatcatcacttaatcaatgttgccgctgcctccttgcagatatggccctcacttgtcgccttcgcgttcccatcactggttaccgaggaagaaactcgcgccgtagaagagggatgttgggacgcggaatgcgacgctacaggcgacggcgtgctatccgcaagcaattgcggggtggttttttaccagccttaattccaattatcgctgctgcaattggcgcgataccaggcatagcttccgtggcggttcaggcctcgcaacgacattgacattggaaaaaaaacgtataaataaaaaaaaatacaatggactctgacactcctggtcctgtgactatgttttcttagagatggaagacatcaatttttcatccttggctccgcgacacggcacgaagccgtacatgggcacctggagcgacatcggcacgagccaactgaacgggggcgccttcaattggagcagtatctggagcgggcttaaaaattttggctcaaccataaaaacatacgggaacaaagcttggaacagcagtacaggacaggcgcttagaaataaacttaaagaccagaacttccaacaaaaagtagtcgatgggatagcttccggcatcaatggagtggtagatttggctaaccaggctgtgcagaaaaagataaacagtcgtttggacccgccgccagcaaccccaggtgaaatgcaagtggaggaagaaattcctccgccagaaaaacgaggcgacaagcgtccgcgtcccgatttggaagagacgctggtgacgcgcgtagatgaaccgccttcttatgaggaagcaacgaagcttggaatgcccaccactagaccgatagccccaatggccaccggggtgatgaaaccttctcagttgcatcgacccgtcaccttggatttgccccctccccctgctgctactgctgtacccgcttctaagcctgtcgctgccccgaaaccagtcgccgtagccaggtcacgtcccgggggcgctcctcgtccaaatgcgcactggcaaaatactctgaacagcatcgtgggtctaggcgtgcaaagtgtaaaacgccgtcgctgcttttaattaaatatggagtagcgcttaacttgcctatctgtgtatatgtgtcattacacgccgtcacagcagcagaggaaaaaaggaagaggtcgtgcgtcgacgctgagttactttcaagatggccaccccatcgatgctgccccaatgggcatacatgcacatcgccggacaggatgcttcggagtacctgagtccgggtctggtgcagttcgcccgcgccacagacacctacttcaatctgggaaataagtttagaaatcccaccgtagcgccgacccacgatgtgaccaccgaccgtagccagcggctcatgttgcgcttcgtgcccgttgaccgggaggacaatacatactcttacaaagtgcggtacaccctggccgtgggcgacaacagagtgctggatatggccagcacgttctttgacattaggggcgtgttggacagaggtcccagtttcaaaccctattctggtacggcttacaactctctggctcctaaaggcgctccaaatgcatctcaatggattgcaaaaggcgtaccaactgcagcagccgcaggcaatggtgaagaagaacatgaaacagaggagaaaactgctacttacacttttgccaatgctcctgtaaaagccgaggctcaaattacaaaagagggcttaccaataggtttggagatttcagctgaaaacgaatctaaacccatctatgcagataaactttatcagccagaacctcaagtgggagatgaaacttggactgacctagacggaaaaaccgaagagtatggaggcagggctctaaagcctactactaacatgaaaccctgttacgggtcctatgcgaagcctactaatttaaaaggtggtcaggcaaaaccgaaaaactcggaaccgtcgagtgaaaaaattgaatatgatattgacatggaattttttgataactcatcgcaaagaacaaacttcagtcctaaaattgtcatgtatgcagaaaatgtaggtttggaaacgccagacactcatgtagtgtacaaacctggaacagaagacacaagttccgaagctaatttgggacaacagtctatgcccaacagacccaactacattggcttcagagataactttattggactcatgtactataacagtactggtaacatgggggtgctggctggtcaagcgtctcagttaaatgcagtggttgacttgcaggacagaaacacagaactttcttaccaactcttgcttgactctctgggcgacagaaccagatactttagcatgtggaatcaggctgtggacagttatgatcctgatgtacgtgttattgaaaatcatggtgtggaagatgaacttcccaactattgttttccactggacggcataggtgttccaacaaccagttacaaatcaatagttccaaatggagaagataataataattggaaagaacctgaagtaaatggaacaagtgagatcggacagggtaatttgtttgccatggaaattaaccttcaagccaatctatggcgaagtttcctttattccaatgtggctctgtatctcccagactcgtacaaatacaccccgtccaatgtcactcttccagaaaacaaaaacacctacgactacatgaacgggcgggtggtgccgccatctctagtagacacctatgtgaacattggtgccaggtggtctctggatgccatggacaatgtcaacccattcaaccaccaccgtaacgctggcttgcgttaccgatctatgcttctgggtaacggacgttatgtgcctttccacatacaagtgcctcaaaaattcttcgctgttaaaaacctgctgcttctcccaggctcctacacttatgagtggaactttaggaaggatgtgaacatggttctacagagttccctcggtaacgacctgcgggtagatggcgccagcatcagtttcacgagcatcaacctctatgctacttttttccccatggctcacaacaccgcttccacccttgaagccatgctgcggaatgacaccaatgatcagtcattcaacgactacctatctgcagctaacatgctctaccccattcctgccaatgcaaccaatattcccatttccattccttctcgcaactgggcggctttcagaggctggtcatttaccagactgaaaaccaaagaaactccctctttggggtctggatttgacccctactttgtctattctggttctattccctacctggatggtaccttctacctgaaccacacttttaagaaggtttccatcatgtttgactcttcagtgagctggcctggaaatgacaggttactatctcctaacgaatttgaaataaagcgcactgtggatggcgaaggctacaacgtagcccaatgcaacatgaccaaagactggttcttggtacagatgctcgccaactacaacatcggctatcagggcttctacattccagaaggatacaaagatcgcatgtattcatttttcagaaacttccagcccatgagcaggcaggtggttgatgaggtcaattacaaagacttcaaggccgtcgccataccctaccaacacaacaactctggctttgtgggttacatggctccgaccatgcgccaaggtcaaccctatcccgctaactatccctatccactcattggaacaactgccgtaaatagtgttacgcagaaaaagttcttgtgtgacagaaccatgtggcgcataccgttctcgagcaacttcatgtctatgggggcccttacagacttgggacagaatatgctctatgccaactcagctcatgctctggacatgacctttgaggtggatcccatggatgagcccaccctgctttatcttctcttcgaagttttcgacgtggtcagagtgcatcagccacaccgcggcatcatcgaggcagtctacctgcgtacaccgttctcggccggtaacgctaccacgtaagaagcttcttgcttcttgcaaatagcagctgcaaccatggcctgcggatcccaaaacggctccagcgagcaagagctcagagccattgtccaagacctgggttgcggaccctattttttgggaacctacgataagcgcttcccggggttcatggcccccgataagctcgcctgtgccattgtaaatacggccggacgtgagacggggggagagcactggttggctttcggttggaacccacgttctaacacctgctacctttttgatccttttggattctcggatgatcgtctcaaacagatttaccagtttgaatatgagggtctcctgcgccgcagcgctcttgctaccaaggaccgctgtattacgctggaaaaatctacccagaccgtgcagggcccccgttctgccgcctgcggacttttctgctgcatgttccttcacgcctttgtgcactggcctgaccgtcccatggacggaaaccccaccatgaaattgctaactggagtgccaaacaacatgcttcattctcctaaagtccagcccaccctgtgtgacaatcaaaaagcactctaccattttcttaatacccattcgccttattttcgctctcatcgtacacacatcgaaagggccactgcgttcgaccgtatggatgttcaataatgactcatgtaaacaacgtgttcaataaacatcactttatttttttacatgtatcaaggctctggattacttatttatttacaagtcgaatgggttctgacgagaatcagaatgacccgcaggcagtgatacgttgcggaactgatacttgggttgccacttgaattcgggaatcaccaacttgggaaccggtatatcgggcaggatgtcactccacagctttctggtcagctgcaaagctccaagcaggtcaggagccgaaatcttgaaatcacaattaggaccagtgctctgagcgcgagagttgcggtacaccggattgcagcactgaaacaccatcagcgacggatgtctcacgcttgccagcacggtgggatctgcaatcatgcccacatccagatcttcagcattggcaatgctgaacggggtcatcttgcaggtctgcctacccatggcgggcacccaattaggcttgtggttgcaatcgcagtgcagggggatcagtatcatcttggcctgatcctgtctgattcctggatacacggctctcatgaaagcatcatattgcttgaaagcctgctgggctttactaccctcggtataaaacatcccgcaggacctgctcgaaaactggttagctgcacagccggcatcattcacacagcagcgggcgtcattgttggctatttgcaccacacttctgccccagcggttttgggtgattttggttcgctcgggattctcctttaaggctcgttgtccgttctcgctggccacatccatctcgataatctgctccttctgaatcataatattgccatgcaggcacttcagcttgccctcataatcattgcagccatgaggccacaacgcacagcctgtacattcccaattatggtgggcgatctgagaaaaagaatgtatcattccctgcagaaatcttcccatcatcgtgctcagtgtcttgtgactagtgaaagttaactggatgcctcggtgctcttcgtttacgtactggtgacagatgcgcttgtattgttcgtgttgctcaggcattagtttaaaacaggttctaagttcgttatccagcctgtacttctccatcagcagacacatcacttccatgcctttctcccaagcagacaccaggggcaagctaatcggattcttaacagtgcaggcagcagctcctttagccagagggtcatctttagcgatcttctcaatgcttcttttgccatccttctcaacgatgcgcacgggcgggtagctgaaacccactgctacaagttgcgcctcttctctttcttcttcgctgtcttgactgatgtcttgcatggggatatgtttggtcttccttggcttctttttggggggtatcggaggaggaggactgtcgctccgttccggagacagggaggattgtgacgtttcgctcaccattaccaactgactgtcggtagaagaacctgaccccacacggcgacaggtgtttttcttcgggggcagaggtggaggcgattgcgaagggctgcggtccgacctggaaggcggatgactggcagaaccccttccgcgttcgggggtgtgctccctgtggcggtcgcttaactgatttccttcgcggctggccattgtgttctcctaggcagagaaacaacagacatggaaactcagccattgctgtcaacatcgccacgagtgccatcacatctcgtcctcagcgacgaggaaaaggagcagagcttaagcattccaccgcccagtcctgccaccacctctaccctagaagataaggaggtcgacgcatctcatgacatgcagaataaaaaagcgaaagagtctgagacagacatcgagcaagacccgggctatgtgacaccggtggaacacgaggaagagttgaaacgctttctagagagagaggatgaaaactgcccaaaacagcgagcagataactatcaccaagatgctggaaatagggatcagaacaccgactacctcatagggcttgacggggaagacgcgctccttaaacatctagcaagacagtcgctcatagtcaaggatgcattattggacagaactgaagtgcccatcagtgtggaagagctcagctgcgcctacgagcttaaccttttttcacctcgtactccccccaaacgtcagccaaacggcacctgcgagccaaatcctcgcttaaacttttatccagcttttgctgtgccagaagtactggctacctatcacatcttttttaaaaatcaaaaaattccagtctcctgccgcgctaatcgcacccgcgccgatgccctactcaatctgggacctggttcacgcttacctgatatagcttccttggaagaggttccaaagatcttcgagggtctgggcaataatgagactcgggccgcaaatgctctgcaaaagggagaaaatggcatggatgagcatcacagcgttctggtggaattggaaggcgataatgccagactcgcagtactcaagcgaagcgtcgaggtcacacacttcgcatatcccgctgtcaacctgccccctaaagtcatgacggcggtcatggaccagttactcattaagcgcgcaagtcccctttcagaagacatgcatgacccagatgcctgtgatgagggtaaaccagtggtcagtgatgagcagctaacccgatggctgggcaccgactctccccgggatttggaagagcgtcgcaagcttatgatggccgtggtgctggttaccgtagaactagagtgtctccgacgtttctttaccgattcagaaaccttgcgcaaactcgaagagaatctgcactacacttttagacacggctttgtgcggcaggcatgcaagatatctaacgtggaactcaccaacctggtttcctacatgggtattctgcatgagaatcgcctaggacaaagcgtgctgcacagcacccttaagggggaagcccgccgtgattacatccgcgattgtgtctatctctacctgtgccacacgtggcaaaccggcatgggtgtatggcagcaatgtttagaagaacagaacttgaaagagcttgacaagctcttacagaaatctcttaaggttctgtggacagggttcgacgagcgcaccgtcgcttccgacctggcagacctcatcttcccagagcgtctcagggttactttgcgaaacggattgcctgactttatgagccagagcatgcttaacaattttcgctctttcatcctggaacgctccggtatcctgcccgccacctgctgcgcactgccctccgactttgtgcctctcacctaccgcgagtgccccccgccgctatggagtcactgctacctgttccgtctggccaactatctctcctaccactcggatgtgatcgaggatgtgagcggagacggcttgctggagtgccactgccgctgcaatctgtgcacgccccaccggtccctagcttgcaacccccagttgatgagcgaaacccagataataggcacctttgaattgcaaggccccagcagccaaggcgatgggtcttctcctgggcaaagtttaaaactgaccccgggactgtggacctccgcctacttgcgcaagtttgctccggaagattaccacccctatgaaatcaagttctatgaggaccaatcacagcctccaaaggccgaactttcggcttgcgtcatcacccagggggcaattctggcccaattgcaagccatccaaaaatcccgccaagaatttctactgaaaaagggtaagggggtctaccttgacccccagaccggcgaggaactcaacacaaggttccctcaggatgtcccaacgacgagaaaacaagaagttgaaggtgcagccgccgcccccagaagatatggaggaagattgggacagtcaggcagaggaggcggaggaggacagtctggaggacagtctggaggaagacagtttggaggaggaaaacgaggaggcagaggaggtggaagaagtaaccgccgacaaacagttatcctcggctgcggagacaagcaacagcgctaccatctccgctccgagtcgaggaacccggcggcgtcccagcagtagatgggacgagaccggacgcttcccgaacccaaccagcgcttccaagaccggtaagaaggatcggcagggatacaagtcctggcgggggcataagaatgccatcatctcctgcttgcatgagtgcgggggcaacatatccttcacgcggcgctacttgctattccaccatggggtgaactttccgcgcaatgttttgcattactaccgtcacctccacagcccctactatagccagcaaatcccgacagtctcgacagataaagacagcggcggcgacctccaacagaaaaccagcagcggcagttagaaaatacacaacaagtgcagcaacaggaggattaaagattacagccaacgagccagcgcaaacccgagagttaagaaatcggatctttccaaccctgtatgccatcttccagcagagtcggggtcaagagcaggaactgaaaataaaaaaccgatctctgcgttcgctcaccagaagttgtttgtatcacaagagcgaagatcaacttcagcgcactctcgaggacgccgaggctctcttcaacaagtactgcgcgctgactcttaaagagtaggcagcgaccgcgcttattcaaaaaaggcgggaattacatcatcctcgacatgagtaaagaaattcccacgccttacatgtggagttatcaaccccaaatgggattggcagcaggcgcctcccaggactactccacccgcatgaattggctcagcgccgggccttctatgatttctcgagttaatgatatacgcgcctaccgaaaccaaatacttttggaacagtcagctcttaccaccacgccccgccaacaccttaatcccagaaattggcccgccgccctagtgtaccaggaaagtcccgctcccaccactgtattacttcctcgagacgcccaggccgaagtccaaatgactaatgcaggtgcgcagttagctggcggctccaccctatgtcgtcacaggcctcggcataatataaaacgcctgatgatcagaggccgaggtatccagctcaacgacgagtcggtgagctctccgcttggtctacgaccagacggaatctttcagattgccggctgcgggagatcttccttcacccctcgtcaggctgttctgactttggaaagttcgtcttcgcaaccccgctcgggcggaatcgggaccgttcaatttgtagaggagtttactccctctgtctacttcaaccccttctccggatctcctgggcactacccggacgagttcataccgaacttcgacgcgattagcgagtcagtggacggctacgattgatgtctggtgacgcggctgagctatctcggctgcgacatctagaccactgccgccgctttcgctgctttgcccgggaacttattgagttcatctacttcgaactccccaaggatcaccctcaaggtccggcccacggagtgcggattactatcgaaggcaaaatagactctcgcctgcaacgaattttctcccagcggcccgtgctgatcgagcgagaccagggaaacaccacggtttccatctactgcatttgtaatcaccccggattgcatgaaagcctttgctgtcttatgtgtactgagtttaataaaaactgaattaagactctcctacggactgccgcttcttcaacccggattttacaaccagaagaacaaaacttttcctgtcgtccaggactctgttaacttcacctttcctactcacaaactagaagctcaacgactacaccgcttttccagaagcattttccctactaatactactttcaaaaccggaggtgagctccacggtctccctacagaaaacccttgggtggaagcgggccttgtagtactaggaattcttgcgggtgggcttgtgattattctttgctacctatacacaccttgcttcactttcctagtggtgttgtggtattggtttaaaaaatggggcccatactagtcttgcttgttttactttcgcttttggaaccgggttctgccaattacgatccatgtctagactttgacccagaaaactgcacacttacttttgcacccgacacaagccgcatctgtggagttcttattaagtgcggatgggaatgcaggtccgttgaaattacacacaataacaaaacctggaacaataccttatccaccacatgggagccaggagttcccgagtggtacactgtctctgtccgaggtcctgacggttccatccgcattagtaacaacactttcattttttctgaaatgtgcgatctggccatgttcatgagcaaacagtattctctatggcctcctagcaaggacaacatcgtaacgttctccattgcttattgcttgtgcgcttgccttcttactgctttactgtgcgtatgcatacacctgcttgtaaccactcgcatcaaaaacgccaataacaaagaaaaaatgccttaacctctttctgtttacagacatggcttctcttacatctctcatatttgtcagcattgtcactgccgctcacggacaaacagtcgtctctatcccactaggacataattacactctcataggacccccaatcacttcagaggtcatctggaccaaactgggaagcgttgattactttgatataatctgtaacaaaacaaaaccaataatagtaacttgcaacatacaaaatcttacattgattaatgttagcaaagtttacagcggttactattatggttatgacagatacagtagtcaatatagaaattacttggttcgtgttacccagttgaaaaccacgaaaatgccaaatatggcaaagattcgatccgatgacaattctctagaaacttttacatctcccaccacacccgacgaaaaaaacatcccagattcaatgattgcaattgttgcagcggtggcagtggtgatggcactaataataatatgcatgcttttatatgcttgtcgctacaaaaagtttcatcctaaaaaacaagatctcctactaaggcttaacatttaatttctttttatacagccatggtttccactaccacattccttatgcttactagtctcgcaactctgacttctgctcgctcacacctcactgtaactataggctcaaactgcacactaaaaggacctcaaggtggtcatgtcttttggtggagaatatatgacaatggatggtttacaaaaccatgtgaccaacctggtagatttttctgcaacggcagagacctaaccattatcaacgtgacagcaaatgacaaaggcttctattatggaaccgactataaaagtagtttagattataacattattgtactgccatctaccactccagcaccccgcacaactactttctctagcagcagtgtcgctaacaatacaatttccaatccaacctttgccgcgcttttaaaacgcactgtgaataattctacaacttcacatacaacaatttccacttcaacaatcagcatcatcgctgcagtgacaattggaatatctattcttgtttttaccataacctactacgcctgctgctatagaaaagacaaacataaaggtgatccattacttagatttgatatttaatttgttctttttttttatttacagtatggtgaacaccaatcatggtacctagaaatttcttcttcaccatactcatctgtgcttttaatgtttgcgctactttcacagcagtagccacagcaaccccagactgtataggagcatttgcttcctatgcactttttgcttttgttacttgcatctgcgtatgtagcatagtctgcctggttattaattttttccaacttctagactggatccttgtgcgaattgcctacctgcgccaccatcccgaataccgcaaccaaaatatcgcggcacttcttagactcatctaaaaccatgcaggctatactaccaatatttttgcttctattgcttccctacgctgtctcaaccccagctgcctatagtactccaccagaacaccttagaaaatgcaaattccaacaaccgtggtcatttcttgcttgctatcgagaaaaatcagaaatccccccaaatttaataatgattgctggaataattaatataatctgttgcaccataatttcatttttgatataccccctatttgattttggctggaatgctcccaatgcacatgatcatccacaagacccagaggaacacattcccccacaaaacatgcaacatccaatagcgctaatagattacgaaagtgaaccacaacccccactactccctgctattagttacttcaacctaaccggcggagatgactgaaacactcaccacctccaattccgccgaggatctgctcgatatggacggccgcgtctcagaacaacgacttgcccaactacgcatccgccagcagcaggaacgcgtggccaaagagctcagagatgtcatccaaattcaccaatgcaaaaaaggcatattctgtttggtaaaacaagccaagatatcctacgagatcaccgctactgaccatcgcctctcttacgaacttggcccccaacgacaaaaatttacctgcatggtgggaatcaaccccatagttatcacccaacaaagtggagatactaagggttgcattcactgctcctgcgattccatcgagtgcacctacaccctgctgaagaccctatgcggcctaagagacctgctaccaatgaattaaaaaaaaatgattaataaaaaatcacttacttgaaatcagcaataaggtctctgttgaaattttctcccagcagcacctcacttccctcttcccaactctggtattctaaaccccgttcagcggcatactttctccatactttaaaggggatgtcaaattttagctcctctcctgtacccacaatcttcatgtctttcttcccagatgaccaagagagtccggctcagtgactccttcaaccctgtctacccctatgaagatgaaagcacctcccaacacccctttataaacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaaaataaagtttaagtgtttttatttaaaatcacaaaattcgagtagttattttgcctccaccttcccatttgacagaatacaccaatctctccccacgcacagctttaaacatttggataccattagagatagacattgttttagattccacattccaaacagtttcagagcgagccaatctggggtcagtgatagataaaaatccatcgcgatagtcttttaaagcgctttcacagtccaactgctgcggatgcgactccggagtttggatcacggtcatctggaagaagaacgatgggaatcataatccgaaaacggtatcggacgattgtgtctcatcaaacccacaagcagccgctgtctgcgtcgctccgtgcgactgctgtttatgggatcagggtccacagtttcctgaagcatgattttaatagcccttaacatcaactttctggtgcgatgcgcgcagcaacgcattctgatttcactcaaatctttgcagtaggtacaacacattattacaatattgtttaataaaccataattaaaagcgctccagccaaaactcatatctgatataatcgcccctgcatgaccatcataccaaagtttaatataaattaaatgacgttccctcaaaaacacactacccacatacatgatctcttttggcatgtgcatattaacaatctgtctgtaccatggacaacgttggttaatcatgcaacccaatataaccttccggaaccacactgccaacaccgctcccccagccatgcattgaagtgaaccctgctgattacaatgacaatgaagaacccaattctctcgaccgtgaatcacttgagaatgaaaaatatctatagtggcacaacatagacataaatgcatgcatcttctcataatttttaactcctcaggatttagaaacatatcccagggaataggaagctcttgcagaacagtaaagctggcagaacaaggaagaccacgaacacaacttacactatgcatagtcatagtatcacaatctggcaacagcgggtggtcttcagtcatagaagctcgggtttcattttcctcacaacgtggtaactgggctctggtgtaagggtgatgtctggcgcatgatgtcgagcgtgcgcgcaaccttgtcataatggagttgcttcctgacattctcgtattttgtatagcaaaacgcggccctggcagaacacactcttcttcgccttctatcctgccgcttagcgtgttccgtgtgatagttcaagtacagccacactcttaagttggtcaaaagaatgctggcttcagttgtaatcaaaactccatcgcatctaattgttctgaggaaatcatccacggtagcatatgcaaatcccaaccaagcaatgcaactggattgcgtttcaagcaggagaggagagggaagagacggaagaaccatgttaatttttattccaaacgatctcgcagtacttcaaattgtagatcgcgcagatggcatctctcgcccccactgtgttggtgaaaaagcacagctaaatcaaaagaaatgcgattttcaaggtgctcaacggtggcttccaacaaagcctccacgcgcacatccaagaacaaaagaataccaaaagaaggagcattttctaactcctcaatcatcatattacattcctgcaccattcccagataattttcagctttccagccttgaattattcgtgtcagttcttgtggtaaatccaatccacacattacaaacaggtcccggagggcgccctccaccaccattcttaaacacaccctcataatgacaaaatatcttgctcctgtgtcacctgtagcgaattgagaatggcaacatcaattgacatgcccttggctctaagttcttctttaagttctagttgtaaaaactctctcatattatcaccaaactgcttagccagaagccccccgggaacaagagcaggggacgctacagtgcagtacaagcgcagacctccccaattggctccagcaaaaacaagattggaataagcatattgggaaccaccagtaatatcatcgaagttgctggaaatataatcaggcagagtttcttgtagaaattgaataaaagaaaaatttgccaaaaaaacattcaaaacctctgggatgcaaatgcaataggttaccgcgctgcgctccaacattgttagttttgaattagtctgcaaaaataaaaaaaaaacaagcgtcatatcatagtagcctgacgaacaggtggataaatcagtctttccatcacaagacaagccacagggtctccagctcgaccctcgtaaaacctgtcatcgtgattaaacaacagcaccgaaagttcctcgcggtgaccagcatgaataagtcttgatgaagcatacaatccagacatgttagcatcagttaaggagaaaaaacagccaacatagcctttgggtataattatgcttaatcgtaagtatagcaaagccacccctcgcggatacaaagtaaaaggcacaggagaataaaaaatataattatttctctgctgctgtttaggcaacgtcgcccccggtccctctaaatacacatacaaagcctcatcagccatggcttaccagagaaagtacagcgggcacacaaaccacaagctctaaagtcactctccaacctctccacaatatatatacacaagccctaaactgacgtaatgggactaaagtgtaaaaaatcccgccaaacccaacacacaccccgaaactgcgtcaccagggaaaagtacagtttcacttccgcaatcccaacaagcgtcacttcctctttctcacggtacgtcacatcccattaacttacaacgtcattttcccacggccgcgccgccccttttaaccgttaaccccacagccaatcaccacacggcccacactttttaaaatcacctcatttacatattggcaccattccatctataaggtatattattgatgatg